(19) United States
(12) Patent Application Publication
(10) Pub. No.: US 2024/0041779 A1
(43) Pub. Date: Feb. 8, 2024

(54) DELAYED SUSTAINED-RELEASE ORAL DRUG DOSAGE FORMS OF A JANUS KINASE (JAK) INHIBITOR AND METHODS OF USE

(71) Applicant: Triastek, Inc., Nanjing (CN)

(72) Inventors: Feihuang DENG, Nanjing (CN); Yu ZHENG, Nanjing (CN); Xin LIU, Nanjing (CN); Qing LUO, Nanjing (CN); Jie CHENG, Nanjing (CN); Luo WANG, Nanjing (CN); Senping CHENG, Nanjing (CN); Xiaoling LI, Dublin, CA (US)

(21) Appl. No.: 18/256,035

(22) PCT Filed: Dec. 8, 2021
(86) PCT No.: PCT/CN2021/136353

§ 371 Date: Jun. 5, 2023

(30) Foreign Application Priority Data
Dec. 8, 2020 (WO) PCT/CN2020/134653

(51) Int. Cl. A61K 9/48; A61K 31/519; B33Y 10/00

(52) U.S. Cl. CPC A61K 9/4833; A61K 31/519; B33Y 10/00; A61K 9/4891; A61K 9/4866; A61K 9/4858; A61K 9/485; A61K 9/0053

(57) ABSTRACT

Provided are delayed sustained-release oral drug dosage forms comprising a Janus kinase (JAK) inhibitor, such as tofacitinib. In other aspects, provided are methods of designing, methods of making, such as using three-dimensional printing, and methods of treatment and/or prevention associated with the oral drug dosage forms described herein.

### CROSS-REFERENCE TO RELATED APPLICATIONS

This application claims the priority benefit of International Application No. PCT/CN2020/134653, filed on Dec. 8, 2020, which is incorporated herein by reference in its entirety.

### TECHNICAL FIELD

The present disclosure, in some aspects, is directed to delayed sustained-release oral drug dosage forms comprising a Janus kinase (JAK) inhibitor, such as tofacitinib. In other aspects, the present disclosure is directed to methods of designing, methods of making, such as using three-dimensional printing, and methods of treatment and/or prevention associated with the oral drug dosage forms described herein.

### BACKGROUND

The Janus kinase-signal transducer and activator of transcription proteins (JAK-STAT) signaling pathway comprises many members, including those of the Janus kinase family of enzymes, and is involved with many fundamental biological processing such as apoptosis, inflammation, and autoimmunity. Members associated with the JAK-STAT signaling pathway have been described, e.g., see, Rawlings et al., J Cell Sci, 117, 2004; and Schwartz et al., Nat Rev Drug Discov, 17, 2017. Dysfunction of the JAK-STAT signaling pathway has been implicated in many human diseases, including cancers and immune system-related diseases such as rheumatoid arthritis, psoriatic arthritis, ulcerative colitis, and psoriasis. Many of these disease are not curable, and treatment merely consists of approaches that try to lessen the impact of associated symptoms. For example, patients with rheumatoid arthritis and psoriatic arthritis often suffer from morning stiffness caused by a period of inactivity during sleep. Similarly, patients with ulcerative colitis often suffer from more severe symptoms early in the morning.

All references cited herein, including patent applications and publications, are incorporated by reference in their entirety.

### BRIEF SUMMARY

In some aspects, provided herein is a delayed sustained-release oral drug dosage form of a Janus kinase (JAK) inhibitor, the delayed sustained-release oral drug dosage form comprising: a sustained-release drug component comprising a first erodible material admixed with the JAK inhibitor; and a delay component, wherein the delay component prevents the release of the JAK inhibitor from the delayed sustained-release oral drug dosage form for about 2 hours to about 6 hours after administration of the delayed sustained-release oral drug dosage form to a human individual.

In some embodiments, the delay component comprises: a delay member comprising a second erodible material not admixed with the JAK inhibitor; and a shell, wherein the delay component completely surrounds the sustained-release drug component. In some embodiments, the sustained-release drug component is a layer having a top surface and a bottom surface. In some embodiments, the top surface of the sustained-release drug component is not flat. In some embodiments, the thickness as measured between the top surface and the bottom surface is substantially consistent.

In some embodiments, the sustained-release drug component is embedded in the shell such that the bottom surface and a side surface of the sustained-release drug component are in direct contact with the shell.

In some embodiments, the top surface of the sustained-release drug component is not in direct contact with the shell.

In some embodiments, the delay member is a layer having a top surface and a bottom surface. In some embodiments, the top surface of the delay member is not flat. In some embodiments, the thickness as measured between the top surface and the bottom surface is substantially consistent.

In some embodiments, the bottom surface of the delay member, or a portion thereof, is in direct contact with the top layer of the sustained-release drug component.

In some embodiments, a side of the delay member is in direct contact with the shell.

In some embodiments, a portion of the bottom surface of the delay member is in direct contact with the shell. In some embodiments, the portion of the bottom surface of the delay member that is in direct contact with the shell forms a perimeter extending beyond the top surface of the sustained-release drug component.

In some embodiments, the delay member and the shell are configured such that the JAK inhibitor is prevented from being released from the delayed sustained-release oral drug dosage form until after the delay member is eroded.

In some embodiments, the shell comprises an insulating material that is impermeable to bodily fluids. In some embodiments, the insulating material is a non-erodible material. In some embodiments, the insulating material is an erodible material having a pH-sensitive erosion and/or an erosion rate that allows for the complete release of the JAK inhibitor from the delayed sustained-release oral drug dosage form prior to exposure of the sustained-release drug component to bodily fluids due to erosion of the shell.

In some embodiments, the delayed sustained-release oral drug dosage form has a substantially planar top surface. In some embodiments, the top surface is formed by the delay member and the shell. In some embodiments, the shell comprises an inset having a depth, wherein the delay member is configured to fit in the inset of the shell. In some embodiments, the thickness of the delay member is the same as the depth of the inset of the shell. In some embodiments, the top surface is a capsule shape.

In some embodiments, the top surface of the sustained-release drug component is a capsule shape.

In some embodiments, the top surface of the delay member is a capsule shape.

In some embodiments, the delay component prevents the release of the JAK inhibitor from the delayed sustained-release oral drug dosage form for about 2 hours to about 4 hours after administration of the delayed sustained-release oral drug dosage form to a human individual.

In some embodiments, the delay component prevents the release of the JAK inhibitor from the delayed sustained-release oral drug dosage form for about 2 hours to about 3 hours after administration of the delayed sustained-release oral drug dosage form to a human individual.

In some embodiments, the sustained-release drug component is configured to release the JAK inhibitor from the delayed sustained-release oral drug dosage form according to the following: (i) not more than 30% of the total JAK inhibitor is released at 1 hour after complete erosion of the delay component or a portion thereof; (ii) not less than 35% and not more than 75% of the total JAK inhibitor is released at 2.5 hours after complete erosion of the delay component or a portion thereof; and (iii) not less than 75% of the total JAK inhibitor is released at 5 hours after complete erosion of the delay component or a portion thereof.

In some embodiments, the release of the JAK inhibitor is based on an in vitro release rate.

In some embodiments, when administered to the human individual, the mean area under the plasma concentration versus time curve after complete erosion of the delay component or a portion thereof is about 17 ng-hr/mL per mg JAK inhibitor dosed to about 42 ng-hr/mL per mg of JAK inhibitor dosed.

In some embodiments, the Tₘₐₓ occurs within about 6 hours after complete erosion of the delay component or a portion thereof.

In some embodiments, when administered to the human individual, the ratio of geometric mean plasma Cₘₐₓ to Cₘᵢₙ is about 10 to about 100.

In some embodiments, the release of the JAK inhibitor is based on an in vitro dissolution technique comprising use of a USP rotating paddle apparatus rotated at about 50 RPM and a test medium comprising 900 mL of 0.05 M potassium phosphate buffer at pH 6.8 and 37° C.

In some embodiments, the top surface of the sustained-release drug component has a surface area of about 20 mm² to about 400 mm².

In some embodiments, the top surface of the sustained-release drug component has a largest crossing dimension of about 5 mm to about 20 mm.

In some embodiments, the top surface of the sustained-release drug component has a crossing dimension perpendicular to a largest crossing dimension of about 2 mm to about 20 mm.

In some embodiments, the sustained-release drug component has a thickness of about 0.2 mm to about 5 mm.

In some embodiments, the sustained-release drug component has a drug mass fraction (m_{F}) of the JAK inhibitor of about 0.2 to about 0.6.

In some embodiments, the sustained-release drug layer has an in vitro dissolution rate of about 2% per hour to about 40% per hour based on an in vitro dissolution technique comprising use of a USP rotating paddle apparatus rotated at about 50 RPM and a test medium comprising 900 mL of 0.05 M potassium phosphate buffer at pH 6.8 and 37° C.

In some embodiments, the first erodible material of the sustained-release drug component comprises one or more of hydroxypropyl cellulose (HPC EF), vinylpyrrolidone-vinyl acetate copolymer (VA64), triethyl citrate (TEC), and glycerin.

In some embodiments, the first erodible material of the sustained-release drug component comprises HPC EF at about 35 w/w % to about 45 w/w %, VA64 at about 5 w/w % to about 15 w/w %, and glycerin at about 10 w/w % to about 20 w/w %.

In some embodiments, the top surface of the delay member has a surface area of about 20 mm² to about 400 mm².

In some embodiments, the top surface of the delay member has a largest crossing dimension of about 5 mm to about 20 mm.

In some embodiments, the top surface of the delay member has a crossing dimension perpendicular to a largest crossing dimension of about 2 mm to about 20 mm.

In some embodiments, the delay member has a thickness of about 0.2 mm to about 5 mm.

In some embodiments, the delay completely dissolves with in about 6 hours after administration of the delayed sustained-release oral drug dosage form to the human individual.

In some embodiments, the second erodible material of the delay layer comprises one or more of hydroxypropyl cellulose (HPC EF), triethyl citrate (TEC), and titanium dioxide.

In some embodiments, the delay layer comprises HPC EF at about 80 w/w % to about 90 w/w %, TEC at about 10 w/w % to about 20 w/w %, and titanium dioxide at about 0.1 w/w % to about 0.3 w/w %.

In some embodiments, the shell has a largest crossing dimension of about 5 mm to about 20 mm.

In some embodiments, the shell has a crossing dimension perpendicular to a largest crossing dimension of about 5 mm to about 20 mm.

In some embodiments, the delayed sustained-release oral drug dosage form has a thickness of about 0.2 mm to about 15 mm.

In some embodiments, the shell has a minimum thickness of at least about 0.4 mm.

In some embodiments, the shell comprises one or more of ammonio methacrylate copolymer type B, ethylcellulose, stearic acid, and titanium dioxide.

In some embodiments, the shell comprises ammonio methacrylate copolymer type B at about 60 w/w % to about 70 w/w %, ethylcellulose at about 10 w/w % to about 20 w/w %, stearic acid at about 15 w/w % to about 25 w/w %, and titanium dioxide at about 0.1 w/w % to about 0.3 w/w %.

In some embodiments, the JAK inhibitor interferes with the JAK-STAT signaling pathway. In some embodiments, wherein the JAK inhibitor is an inhibitor of any one or more of JAK1, JAK2, JAK3, or TYK2.

In some embodiments, the JAK inhibitor is tofacitinib or a pharmaceutically acceptable salt thereof. In some embodiments, the JAK inhibitor is tofacitinib citrate.

In some embodiments, the amount of the JAK inhibitor in the delayed sustained-release oral drug dosage form is about 11 mg.

In some embodiments, the amount of the JAK inhibitor in the delayed sustained-release oral drug dosage form is about 22 mg.

In some embodiments, the delayed-sustained-release oral drug dosage form is not an osmotic-controlled release oral drug dosage form.

In other aspects, provided herein is a commercial batch of any delayed sustained-release oral drug dosage form described herein, wherein the commercial batch has a standard deviation of about 0.05 or less for each of the following: an amount of a JAK inhibitor in the delayed sustained-release oral drug dosage form; weight of the delayed sustained-release oral drug dosage form; a largest crossing dimension of the delayed sustained-release oral drug dosage form; and a crossing dimension perpendicular to the largest crossing dimension of the delayed sustained-release oral drug dosage form.

In some embodiments, the commercial batch comprises at least about 1000 of the delayed sustained-release oral drug dosage forms.

In other aspects, provided herein is a method of three-dimensional (3D) printing of any delayed sustained-release oral drug dosage form described herein, the method comprising: (a) dispensing the delay component or a portion thereof; and (b) dispensing the sustained-release drug component comprising the first erodible material admixed with the JAK inhibitor.

In some embodiments, dispensing the delay component comprises: (i) dispensing the shell; and (ii) dispensing the delay member comprising the second erodible material not admixed with the JAK inhibitor.

In some embodiments, the dispensing is via melt extrusion deposition (MED).

In some embodiments, the dispensing of the delay component, dispensing of the shell, and dispensing of the delay member are performed by a different printing head.

In other aspects, provided herein is a method for preparing a delayed sustained-release tofacitinib oral drug dosage form by three-dimensional (3D) printing, wherein the delayed sustained-release tofacitinib oral drug dosage form comprises a shell containing an insoluble material, a pharmaceutical core containing tofacitinib, and a delay member without tofacitinib, the method comprising: (a) dispensing the insoluble material to form the shell; (b) dispensing the core material containing tofacitinib; and (c) dispensing the delay member without tofacitinib.

In some embodiments, the dispensing is via melt extrusion deposition (MED).

In some embodiments, the dispensing of each material is performed by a different printing head.

In other aspects, provided herein is a method of injection molding any oral drug dosage form described herein, the method comprising: (a) injecting a hot melt of the shell material into a mold cavity to form the shell; (b) injecting a hot melt of the first erodible material admixed with a JAK inhibitor into the shell to form the sustained-release drug component; and (c) injecting a hot melt of the second erodible material not admixed with the JAK inhibitor into the shell to form the delay member.

In other aspects, provided herein is a method of injection molding any delayed sustained-release oral drug dosage form described herein, the method comprising: (a) hot melting a shell material, a first erodible material admixed with a JAK inhibitor, and a second erodible material not admixed with the JAK inhibitor; (b) delivering each material to the respective injection unit; (c) injecting a hot melt of the shell material into a mold cavity to form the shell; (d) allowing the shell to cool and opening the mold to release the shell; (e) transferring the shell to a male mold to inject the first erodible material admixed with the JAK inhibitor to form the sustained-release drug component; (f) injection a hot melt of the first erodible material admixed with the JAK inhibitor to form the sustained-release drug component; (g) allowing the sustained-released drug component to cool and opening the mold to release the shell and the sustained-release drug component; (h) transferring the shell and the sustained-release drug component to a male mold to inject the second erodible material not admixed with the JAK inhibitor to form the delay member; (i) injection a holt melt of the second erodible material not admixed with the JAK inhibitor; and (j) ejecting the delayed sustained-release oral drug dosage form.

In some embodiments, the injection unit is selected from the group consisting of a single screw injection unit, a plunger injection unit, and a gear pump injection unit. In some embodiments, step (c) to step (j) are performed in series. In some embodiments, step (c), step (f), and step (i) are performed at the same time. In some embodiments, step (e), step (h), and step (j) are performed at the same time.

In other aspects, provided herein is a method for preventing morning stiffness caused by rheumatoid arthritis, the method comprising administering to a human individual a delayed sustained-release oral drug dosage form described herein, wherein the delayed sustained-release oral drug dosage form is administered within about 1 hour of going to bed.

In other aspects, provided herein is a method for preventing morning stiffness caused by psoriatic arthritis, the method comprising administering to a human individual a delayed sustained-release oral drug dosage form described herein, wherein the delayed sustained-release oral drug dosage form is administered within about 1 hour of going to bed.

In other aspects, provided herein is a method for treating ulcerative colitis, the method comprising administering to a human individual a delayed sustained-release oral drug dosage form described herein.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### BRIEF DESCRIPTION OF THE DRAWINGS

FIGS. 1A-1H show cross-sectional diagrams of exemplary delayed sustained-release oral drug dosage forms.

FIGS. 2A-2D show diagrams of exemplary delayed sustained-release oral drug dosage forms.

FIG. 3 shows a dissolution plot for a delayed sustained-release oral drug dosage form and a commercially available extended release dosage form.

FIG. 4 shows a dissolution plot for a delayed sustained-release oral drug dosage form and a commercially available extended release dosage form.

FIG. 5 shows a mean plasma concentration-time curve following administration of delayed sustained-release oral drug dosage form and a commercially available extended release dosage form.

FIG. 6 shows a mean plasma concentration-time curve following administration of delayed sustained-release oral drug dosage form and a commercially available extended release dosage form.

FIG. 7 shows a dissolution plot for three oral drug dosage forms described herein and a commercially available extended release dosage form.

FIG. 8 shows a mean plasma concentration-time curve following administration of delayed sustained-release oral drug dosage forms and a commercially available extended release dosage form.

FIG. 9 shows a mean plasma concentration-time curve following administration of delayed sustained-release oral drug dosage form, a commercially available extended release dosage form, and a target delayed sustained-release plasma concentration-time curve.

FIG. 10 shows a dissolution plot for a delayed sustained-release oral drug dosage form.

FIGS. 11A-11F show schematics of dosage forms D-I, respectively.

FIG. 12 shows a dissolution plot for a delayed sustained-release oral drug dosage form.

FIG. 13 shows a mean plasma concentration-time curve following administration of delayed sustained-release oral drug dosage form and a commercially available extended release dosage form.

FIG. 14 shows a dissolution plot for two delayed sustained-release oral drug dosage forms.

FIG. 15 shows a mean plasma concentration-time curve following administration of two delayed sustained-release oral drug dosage forms and a commercially available extended release dosage form.

FIG. 16 shows a dissolution plot for two delayed sustained-release oral drug dosage forms.

FIG. 17 shows a mean plasma concentration-time curve following administration of two delayed sustained-release oral drug dosage forms and a commercially available extended release dosage form.

FIG. 18 shows a dissolution plot for three delayed sustained-release oral drug dosage forms.

FIG. 19 shows a mean plasma concentration-time curve following administration of three delayed sustained-release oral drug dosage forms and a commercially available extended release dosage form.

FIG. 20 shows a dissolution plot for three delayed sustained-release oral drug dosage forms.

FIG. 21 shows a mean plasma concentration-time curve following administration of two delayed sustained-release oral drug dosage forms and a commercially available extended release dosage form.

FIG. 22 shows a mean plasma concentration-time curve following administration of a delayed sustained-release oral drug dosage form and a commercially available extended release dosage form.

FIG. 23 shows a dissolution plot for a delayed sustained-release oral drug dosage form.

### DETAILED DESCRIPTION

Provided herein, in some aspects, is a delayed sustained-release oral drug dosage form of a Janus kinase (JAK) inhibitor. In some embodiments, the delayed sustained-release oral drug dosage form comprises a sustained-release drug component comprising the JAK inhibitor, and a delay component, wherein the delay component is configured to prevent the release of the JAK inhibitor from the oral drug dosage form for a desired amount of time after administration of the delayed sustained-release oral drug dosage form to a human individual. In some embodiments, the sustained-release drug component is configured to release the JAK inhibitor according to a desired release profile. In some embodiments, the sustained-release drug component comprises a first erodible material admixed with the JAK inhibitor, wherein release of the JAK inhibitor is based on erosion of the first erodible material.

The delayed sustained-release oral drug dosage forms described herein are based, at least in part, on the inventors' unique insights and findings about the design of such oral drug dosage forms for improving JAK inhibitor treatments via precision drug release. As discussed above, patients with certain dysfunctional JAK-STAT signaling pathway-associated diseases experience severe morning symptoms (or symptoms when awaking from sleep depending on the individual's sleep schedule). The delayed sustained-release oral drug dosage forms described herein are designed and configured such that a human individual can take the oral drug dosage form near bedtime (such as in the evening hours) and awake with reduced symptoms as the oral drug dosage form has released the JAK inhibitor during sleep and the drug level necessary to effectively reduce symptoms is obtained and maintained during this time of need. The delayed sustained-release oral drug dosage forms described herein provide a convenient way to improve treatment compliance with a once-daily administration and improve quality of life during the individual's waking hours.

Thus, in some aspects, provided herein is a delayed sustained-release oral drug dosage form of a Janus kinase (JAK) inhibitor, the delayed sustained-release oral drug dosage form comprising: a sustained-release drug component comprising a first erodible material admixed with the JAK inhibitor; and a delay component, wherein the delay component prevents the release of the JAK inhibitor from the delayed sustained-release oral drug dosage form for about 2 hours to about 6 hours after administration of the delayed sustained-release oral drug dosage form to a human individual.

In other aspects, provided herein is a commercial batch of a delayed sustained-release oral drug dosage form of any one of the delayed sustained-release oral drug dosage forms described herein. In some embodiments, the commercial batch has a standard deviation of about 0.05 or less for any one or more of the following: an amount of a JAK inhibitor in the delayed sustained-release oral drug dosage form; weight of the delayed sustained-release oral drug dosage form; a largest crossing dimension of the delayed sustained-release oral drug dosage form; and a crossing dimension perpendicular to the largest crossing dimension of the delayed sustained-release oral drug dosage form.

In other aspects, provided herein is a method for preparing any one of the delayed sustained-release tofacitinib oral drug dosage forms described herein, the method comprising three-dimensional (3D) printing the delayed sustained-release tofacitinib oral drug dosage. In some embodiments, the delayed sustained-release tofacitinib oral drug dosage form comprises: a sustained-release drug component comprising a first erodible material admixed with the JAK inhibitor; a delay member comprising a second erodible material not admixed with the JAK inhibitor; and a shell comprising an insulating material that is impermeable to bodily fluids, wherein the method comprises: (a) dispensing the sustained-release drug component; (b) dispensing the delay member; and (c) dispensing the shell, to form the delayed sustained-release tofacitinib oral drug dosage.

In other aspects, provided herein is a method for preventing morning stiffness caused by rheumatoid arthritis, the method comprising administering to a human individual a delayed sustained-release oral drug dosage form described herein, wherein the delayed sustained-release oral drug dosage form is administered the evening prior to when the effects are desired to prevent morning stiffness, such as within about 1 hour of going to bed.

In other aspects, provided herein is a method for preventing morning stiffness caused by psoriatic arthritis, the method comprising administering to a human individual a delayed sustained-release oral drug dosage form described herein, wherein the delayed sustained-release oral drug dosage form is administered the evening prior to when the effects are desired to prevent morning stiffness, such as within about 1 hour of going to bed (or an adjusted schedule based on the waking and sleeping hours observed by the individual).

In other aspects, provided herein is a method for treating ulcerative colitis, the method comprising administering to a human individual a delayed sustained-release oral drug dosage form described herein. In some embodiments, the delayed sustained-release oral drug dosage form is administered during the evening hours to provide a reduction of symptoms the following morning.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the term “tofacitinib” includes, unless otherwise indicated, any pharmaceutically acceptable form and salts thereof. In some embodiments, tofacitinib may be present in crystalline form. In some embodiments, tofacitinib may be present in amorphous form. In some embodiments, the pharmaceutically acceptable form is any pharmaceutically acceptable form, including, solvates, hydrates, isomorphs, polymorphs, co-crystals, pseudomorphs, neutral forms, acid addition salt forms, and prodrugs. In some embodiments, the pharmaceutically acceptable form is a pharmaceutically acceptable salt. Conventional concentration and recrystallization techniques may be employed in generating and isolating pharmaceutically acceptable salts of a JAK inhibitor, including use of acids such as acetic acid, lactic acid, succinic acid, maleic acid, tartaric acid, citric acid, gluconic acid, ascorbic acid, mesylic acid, tosylic acid, benzoic acid, cinnamic acid, fumaric acid, sulfuric acid, phosphoric acid, hydrochloric acid, hydrobromic acid, hydroiodic acid, sulfamic acid, sulfonic acid, such as methanesulfonic, benzenesulfonic, and related acids. In some embodiments, tofacitinib is tofacitinib citrate.

As used herein, use of the terms “treat,” “treatment,” “treating,” or equivalents thereof, refer to an approach for obtaining beneficial or desired results including a reduction of symptoms of, e.g., a disease. For purposes of this disclosure, beneficial or desired clinical results include, but are not limited to, one or more of the following: alleviating one or more symptoms resulting from the disease, reducing the severity of one or more symptoms resulting from the disease, preventing the increase in the severity of one or more symptoms resulting from the disease, decreasing the dose of one or more other medications required to treat and/or manage the disease, and increasing the quality of life.

As used herein, use of the terms “prevent,” “prevention,” or “preventing,” or equivalents thereof, refer to an approach for obtaining beneficial or desired results including a reduction in future expected symptoms of, e.g., a disease. For purposes of this disclosure, beneficial or desired clinical results include, but are not limited to, one or more of the following: preventing the occurrence and/or increase in the severity of one or more symptoms resulting from the disease, alleviating one or more symptoms resulting from the disease, reducing the severity of one or more symptoms resulting from the disease, decreasing the dose of one or more other medications required to treat and/or manage the disease, and increasing the quality of life.

As used herein, the term “individual” refers to a mammal and includes, but is not limited to, human, bovine, horse, feline, canine, rodent, rat, mouse, dog, or primate. In some embodiments, the individual is a human individual.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. Delayed Sustained-Release Oral Drug Dosage Forms

Provided herein, in some aspects, is a delayed sustained-release oral drug dosage form of a Janus kinase (JAK) inhibitor. In some embodiments, the delayed sustained-release oral drug dosage forms described herein comprise: a sustained-release drug component comprising the JAK inhibitor; and a delay component, wherein the delay component is configured to prevent the release of the JAK inhibitor from the oral drug dosage form for a desired amount of time after administration of the delayed sustained-release oral drug dosage form to a human individual. In some embodiments, the sustained-release drug component, in conjunction with the oral drug dosage form or a portion thereof, is configured to release the JAK inhibitor according to a desired release profile. In some aspects, the oral drug dosage form further comprises an immediate-release drug component comprising the JAK inhibitor.

The oral drug dosage forms disclosed herein may comprise a variety of combinations of the components described herein, and may be arranged in a diverse array of configurations. Such components, and configurations thereof, for forming a delayed sustained-release oral drug dosage form are configured to achieve the desired delayed sustained-release profile of a JAK inhibitor. In some instances, such components and configurations are described in a modular fashion, and such description is not intended to limit the scope of the oral drug dosage forms encompassed herein.

### A. Components and Configurations of Delayed Sustained-Release Oral Drug Dosage Forms

In some embodiments, the components of the delayed sustained-release oral drug dosage forms described herein include a sustained-release drug component comprising the JAK inhibitor, and a delay component. In some embodiments, the delay component comprises a delay member and a shell.

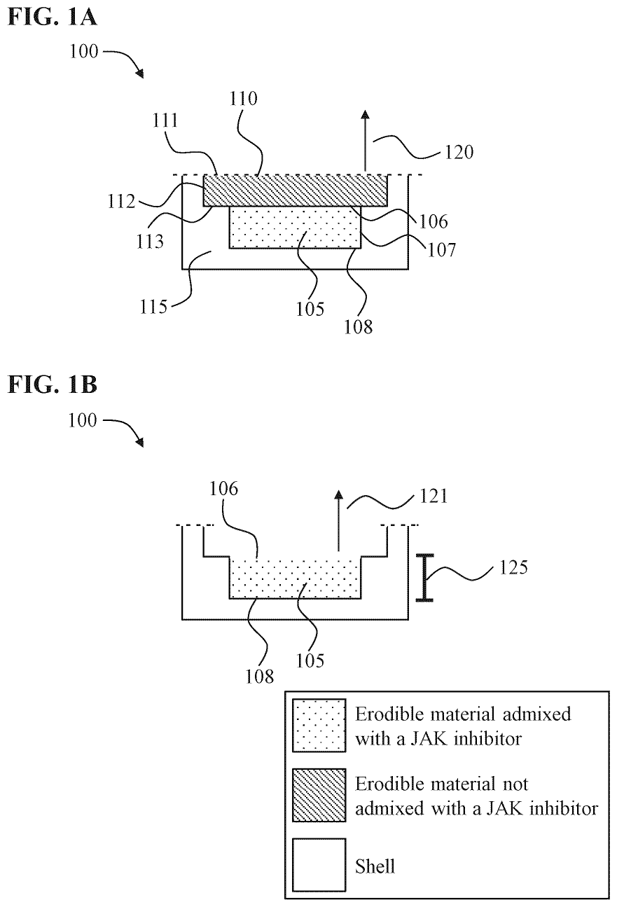

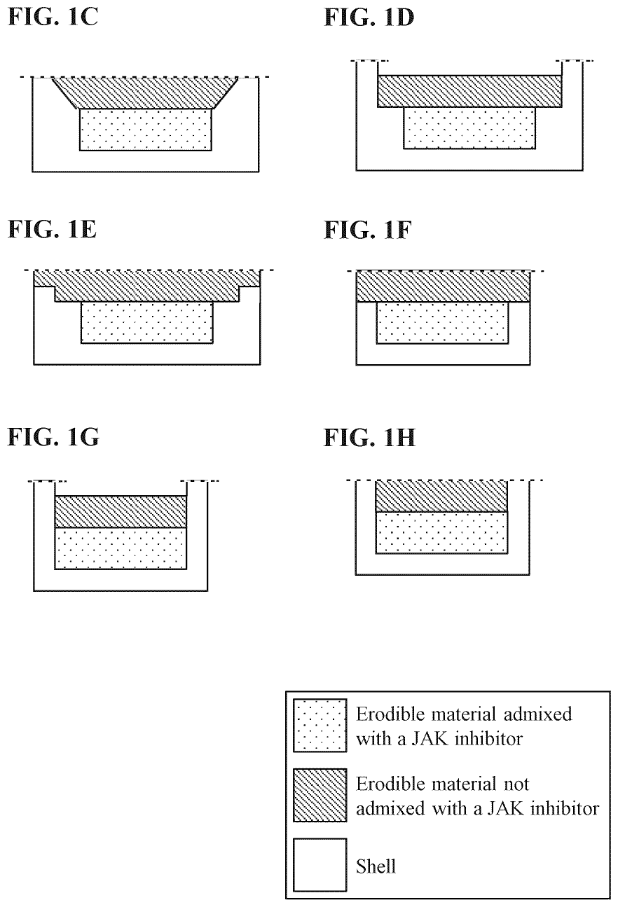

For purposes of illustration and to facilitate the understanding of certain components, and configurations thereof, cross-sectional diagrams of exemplary delayed sustained-release oral drug dosage forms described herein are provided in FIGS. 1A-1H. In some aspects of the present disclosure, as illustrated in FIGS. 1A-1H, the exemplary delayed sustained-release oral drug dosage forms comprise: a sustained-release drug component comprising an erodible material admixed with a JAK inhibitor; and a delay component comprising: a delay member comprising an erodible material not admixed with the JAK inhibitor; and a shell. The dashed lines in FIGS. 1A-1H, represent a portion of one or more components of the delayed sustained-release oral drug dosage forms having an undefined shape in the schematic; such portions of the one or more components may be configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage form. In some embodiments, such portion of the one or more components is not flat.

A cross-sectional diagram of an exemplary delayed sustained-release oral drug dosage form of a Janus kinase (JAK) inhibitor 100 is provided in FIG. 1A, wherein the delayed sustained-release oral drug dosage form 100 comprises: a sustained-release drug component comprising a first erodible material admixed with the JAK inhibitor 105; and a delay component, wherein the delay component comprises: a delay member comprising a second erodible material not admixed with the JAK inhibitor 110; and a shell 115. As shown in FIG. 1A, the sustained-release drug component 105 has a top surface 106, a side surface 107, and a bottom surface 108, and the delay member 110 has a top surface 111, a side surface 112, and a bottom surface 113. In some embodiments, the delay component (e.g., the delay member 110 and the shell 115), surrounds the sustained-release drug component 105. The delayed sustained-release oral drug dosage form 100 of FIG. 1A is configured such that when administered to a human individual, and subjected to a bodily fluid, a top surface 111 of the delay member 110 is first exposed to the bodily fluid and erodes away from the oral drug dosage form in the direction of the arrow 120. As shown in FIG. 1B, at a certain time following administration to the human individual, the top surface 106 of the sustained-release drug component 105 is exposed to the bodily fluid and erodes away from the oral drug dosage form 100 in the direction of the arrow 121. In some embodiments, the thickness of the sustained-release drug component 105 is measured from the top surface 106 to the bottom surface 108 in a direction substantially parallel with the direction of erosion 121 of the sustained-release drug component. For example, as shown in FIG. 1B, the thickness of the sustained-release drug component 105 is indicated by the marker 125.

As disclosed herein, the components of the delayed sustained-release oral drug dosage forms may be configured in numerous shapes and sizes. Unless otherwise specified, reference to certain shapes, sizes, and measurements are reflective of the delayed sustained-release oral drug dosage form prior to administration to a human individual, e.g., prior to erosion of any components thereof.

i. Sustained-Release Drug Components

The delayed sustained-release oral drug dosage forms disclosed herein comprise a sustained-release drug component comprising a JAK inhibitor. In some embodiments, the sustained release drug component comprises an erodible material comprising the JAK inhibitor. The sustained-release drug components may be formed using numerous materials (including materials having a range of JAK inhibitor drug mass fractions) having varying shapes and sizes.

In some embodiments, the sustained-release drug component is a layer. In some embodiments, the sustained-release drug component comprises a plurality of beads.

In some embodiments, the sustained-release drug components are configured having surfaces, such as a surface exposed to a bodily fluid during administration of the oral drug dosage form to a human individual, having a pre-determined shape and surface area. For example, in some embodiments, the sustained-release drug component has a top surface and a bottom surface, wherein the top surface is exposed to a bodily fluid prior to the bottom surface. In some embodiments, the sustained-release drug component is a layer having a top surface and a bottom surface. In some embodiments, the top surface of the sustained-release drug component is not flat, e.g., comprises certain features that extend beyond a top surface plane or surface tolerance threshold (as measured between two parallel planes), such as to reduce adherence of the sustained-release drug layer, or a portion thereof, to an internal body part of the human individual. In some embodiments, the top surface of the sustained-release drug component, or at least a portion thereof, is flat or within a surface tolerance threshold.

The top surface of the sustained-release drug component, as based on the surface exposed to a bodily fluid, can have any shape. In some embodiments, the top surface of the sustained-release drug component, as based on the surface exposed to a bodily fluid, has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the top surface of the sustained-release drug component, such as a sustained-release drug layer, has a surface area of about 10 mm² to about 400 mm², such as any of about 20 mm² to about 200 mm², about 20 mm² to about 100 mm², about 20 mm² to about 60 mm², about 30 mm² to about 50 mm². In some embodiments, the top surface of the sustained-release drug component has a surface area of at least about 20 mm², such as at least about any of 22 mm², 24 mm², 26 mm², 28 mm², 30 mm², 32 mm², 33 mm², 34 mm², 36 mm², 38 mm², 40 mm², 42 mm², 44 mm², 46 mm², 48 mm², 50 mm², 52 mm², 54 mm², 56 mm², 58 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm². In some embodiments, the top surface of the sustained-release drug component has a surface area of less than about 400 mm², such as less than about any of 400 mm², 375 mm², 350 mm², 325 mm², 300 mm², 275 mm², 250 mm², 225 mm², 200 mm², 180 mm², 170 mm², 160 mm², 150 mm², 140 mm², 130 mm², 120 mm², 110 mm², 100 mm², 95 mm 2, 90 mm², 85 mm², 80 mm², 75 mm², 70 mm², 65 mm², 60 mm², 58 mm², 56 mm², 54 mm², 52 mm², 50 mm², 48 mm², 46 mm², 44 mm², 42 mm², 40 mm², 38 mm², 36 mm², 34 mm², 32 mm², 30 mm², 28 mm², 26 mm², 24 mm², 22 mm², or 20 mm². In some embodiments, the top surface of the sustained-release drug component has a surface area of about any of 20 mm², 21 mm², 22 mm², 23 mm², 24 mm², 25 mm², 26 mm², 27 mm², 28 mm², 29 mm², 30 mm², 31 mm², 32 mm², 33 mm², 34 mm², 35 mm², 36 mm², 37 mm², 38 mm², 39 mm², 40 mm², 41 mm², 42 mm², 43 mm², 44 mm², 45 mm², 46 mm², 47 mm², 48 mm², 49 mm², 50 mm², 51 mm², 52 mm², 53 mm², 54 mm², 55 mm², 56 mm², 57 mm², 58 mm², 59 mm², 60 mm², 65 mm², 70 mm², 80 mm², 85 mm², 90 mm², 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm.

In some embodiments, the surface area of the top surface of the sustained-release drug component exposed to a bodily fluid is consistent throughout the thickness of the sustained-release drug component, e.g., as the sustained-release drug component erodes the surface exposed to the bodily fluid has the same surface area. In some embodiments, the surface area of the top surface of the sustained-release drug component exposed to a bodily fluid is different at two or more points, e.g., as the sustained-release drug component erodes the surface exposed to the bodily fluid changes such as increases and/or decreases in surface area during erosion of the sustained-release drug component. In some embodiments, the shape of the surface of the sustained-release drug component exposed to a bodily fluid is consistent throughout the thickness of the sustained-release drug component, e.g., as the sustained-released drug component erodes the surface exposed to the bodily fluid is the same shape. In some embodiments, the shape of the surface of the sustained-release drug component exposed to a bodily fluid is different at two or more points. In some embodiments, the bottom surface of the sustained-release drug component has a surface area that is the same as that of the top surface of the sustained-release drug component. In some embodiments, the bottom surface of the sustained-release drug component has a surface area that is different than that of the top surface of the sustained-release drug component.

In some embodiments, the top surface of the sustained-release drug component, such as a sustained-release drug layer, has a largest crossing dimension of about 5 mm to about 20 mm, such as any of about 5 mm to about 15 mm, about 6 mm to about 13 mm, or about 7 to about 11 mm. In some embodiments, the top surface of the sustained-release drug component has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the top surface of the sustained-release drug component has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the top surface of the sustained-release drug component has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the top surface of the sustained-release drug component, such as a sustained-release drug layer, has a crossing dimension perpendicular to the largest crossing dimension of about 1 mm to about 15 mm, such as any of about 2 mm to about 15 mm, about 2 mm to about 6 mm, or about 1 to about 5 mm. In some embodiments, the top surface of the sustained-release drug component has a crossing dimension perpendicular to the largest crossing dimension of at least about 1 mm, such as at least about any of 2 mm, 3 mm, 4 mm, 5 mm, 6 mm, 7 mm, 8 mm, 9 mm, 10 mm, 11 mm, 12, mm, 13 mm, 14 mm, or 15 mm. In some embodiments, the top surface of the sustained-release drug component has a crossing dimension perpendicular to the largest crossing dimension of less than about 15 mm, such as less than about any of 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the top surface of the sustained-release drug component has a crossing dimension perpendicular to the largest crossing dimension of about any of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm.

In some embodiments, the sustained-release drug component, such as a sustained-release drug component layer, has a thickness of about 0.1 mm to about 5 mm, such as any of about 0.2 mm to about 2 mm, about 0.5 mm to about 1.5 mm, or about 0.8 mm to about 1.4 mm. In some embodiments, the sustained-release drug component has a thickness of at least about 0.1 mm, such as at least about any of 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiments, the sustained-release drug component has a thickness of less than about 5 mm, such as less than about any of 4.8 mm, 4.6 mm, 4.4 mm, 4.2 mm, 4.0 mm, 3.8 mm, 3.6 mm, 3.4 mm, 3.2 mm, 3.0 mm, 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, or 0.1 mm. In some embodiments, the sustained-release drug component has a thickness of about any of 0.1 mm, 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm.

In some embodiments, the sustained-release drug component, such as a sustained-release drug layer, comprises a top surface and a bottom surface, wherein the thickness, as measured between the top surface and the bottom surface, is substantially consistent, such as within a 20% range of an average thickness.

In some embodiments, the sustained-release drug component, such as a sustained-release drug layer, comprises a side surface.

In some embodiments, the sustained-release drug component has a drug mass fraction (m_{F}) of the JAK inhibitor of about 0.1 to about 0.6, such as any of about 0.2 to about 0.5, or about 0.3 to about 0.4. In some embodiments, the sustained-release drug component has a drug mass fraction (m_{F}) of the JAK inhibitor of at least about 0.1, such as at least about any of 0.15, 0.2, 0.25, 0.3, 0.35, 0.4, 0.45, 0.5, 0.55, or 0.6. In some embodiments, the sustained-release drug component has a drug mass fraction (m_{F}) of the JAK inhibitor of less than about 0.6, such as less than about any of 0.55, 0.5, 0.45, 0.4, 0.35, 0.3, 0.25, 0.2, 0.15, or 0.1. In some embodiments, the sustained-release drug component has a drug mass fraction (m_{F}) of the JAK inhibitor of about any of 0.1, 0.15, 0.2, 0.25, 0.3, 0.35, 0.4, 0.45, 0.5, 0.55, or 0.6.

In some embodiments, the sustained-release drug component comprises an erodible material comprising the JAK inhibitor. In some embodiments, the release of the JAK inhibitor from the delayed sustained-release oral drug dosage form is based on the erosion of the sustained-release drug component. In some embodiments, the sustained-release drug component completely erodes, once contacted by bodily fluid in the human individual, over a period of about 3 hours to about 12 hours, such as about 4 hours to about 8 hours, or about 6 hour to about 10 hours. In some embodiments, the sustained-release drug component completely erodes, once contacted by bodily fluid in the human individual, over a period of at least about 3 hours, such as at least about any of 4 hours, 5 hours, 6 hours, 7 hours, 8 hours, 9 hours, 10 hours, 11 hours, or 12 hours. In some embodiments, the sustained-release drug component completely erodes, once contacted by bodily fluid in the human individual, over a period of at less than about 12 hours, such as less than about any of 11 hours, 10 hours, 9 hours, 8 hours, 7 hours, 6 hours, 5 hours, 4 hours, or 3 hours. In some embodiments, the sustained-release drug component completely erodes, once contacted by bodily fluid in the human individual, over a period of about any of 3 hours, 4 hours, 5 hours, 6 hours, 7 hours, 8 hours, 9 hours, 10 hours, 11 hours, or 12 hours.

In some embodiments, the amount of the JAK inhibitor in the sustained-release drug component is about 1 mg to about 50 mg, such as any of about 1 mg to about 25 mg, about 10 mg to about 40 mg, about 10 mg to about 30 mg, about 9 mg to about 12 mg, about 10 mg to about 12 mg, about 19 mg to about 23 mg, or about 21 mg to about 23 mg. In some embodiments, the amount of the JAK inhibitor in the sustained-release drug component is about 1 mg or more, such as about any of 3 mg or more, 4 mg or more, 5 mg or more, 6 mg or more, 7 mg or more, 8 mg or more, 9 mg or more, 10 mg or more, 11 mg or more, 12 mg or more, 13 mg or more, 14 mg or more, 15 mg or more, 16 mg or more, 17 mg or more, 18 mg or more, 19 mg or more, 20 mg or more, 21 mg or more, 22 mg or more, 23 mg or more, 24 mg or more, or 25 mg or more. In some embodiments, the amount of the JAK inhibitor in the sustained-release drug component is about 25 mg or less, such as about any of 24 mg or less, 23 mg or less, 22 mg or less, 21 mg or less, 20 mg or less, 19 mg or less, 18 mg or less, 17 mg or less, 16 mg or less, 15 mg or less, 14 mg or less, 13 mg or less, 12 mg or less, 11 mg or less, 10 mg or less, 9 mg or less, 8 mg or less, 7 mg or less, 6 mg or less, 5 mg or less, 4 mg or less, or 3 mg or less. In some embodiments, the amount of the JAK inhibitor in the sustained-release drug component is about any of 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

In some embodiments, the amount of the JAK inhibitor in the sustained-release drug component is about 11 mg.

In some embodiments, the amount of the JAK inhibitor in the sustained-release drug component is about 22 mg.

In some embodiments, the sustained-release drug component comprises a thermoplastic material, for example a thermoplastic polymer. In some embodiments, the sustained-release drug component comprises a material including any one or more of an erodible thermoplastic material, such as a sustained-release erodible material or an immediate-release erodible material, a drug-diffusion material, a plasticizer, and another additive, e.g., a filler, a binder, a lubricant, a glidant, and a disintegrant.

In some embodiments, the erodible thermoplastic material comprises any one or more of polyvinylpyrrolidone-co-vinyl-acetate (PVP-VA), polyvinylpyrrolidone-polyvinyl acetate copolymer (PVP-VA) 60/40, polyvinylpyrrolidone (PVP), polyethylene oxide (PEO), polyethylene glycol (PEG), polyvinyl alcohol (PVA), polyvinyl caprolactam-polyvinyl acetate-polyethylene glycol graft copolymer 57/30/13, aminoalkyl methacrylate copolymer E, hydroxypropyl methylcellulose acetate succinate or hypromellose acetate succinate (HPMCAS), hydroxypropyl methylcellulose phthalate (HPMCP), copolyvidone, hydroxypropyl cellulose (HPC), hydroxylpropyl methylcellulose or Hypromellose (HPMC), methyl cellulose (MC), methacrylic acid copolymer, poly(dimethylaminoethylmethacrylate-co-methacrylic esters), poly(ethyl acrylate-co-methyl methacrylate-co-trimethylammonioethyl methacrylate chloride), poly(methyl acrylate-co-methyl methacrylate-co-methacrylic acid) 7:3:1, poly(methacrylic acid-co-methylmethacrylate) 1:2, poly(methacylic acid-co-ethyl acrylate) 1:1, poly(methacylic acid-co-methyl methacrylate) 1:1, polyethylene glycol-polyvinyl alcohol graft copolymer 25/75, Kollicoat IR-polyvinyl alcohol 60/40, methacrylic ester copolymer, and ammonioalkyl methacrylate copolymer.

In some embodiments, the sustained-release erodible material comprises any one or more of copolyvidone, polyvinylpyrrolidone-co-vinyl-acetate (PVP-VA), polyvinylpyrrolidone-polyvinyl acetate copolymer (PVP-VA) 60/40, polyvinylpyrrolidone (PVP), hydroxypropyl cellulose (HPC), hydroxylpropyl methylcellulose or Hypromellose (HPMC), hydroxypropyl methylcellulose phthalate (HPMCP), methyl cellulose (MC), methacrylic acid copolymer, poly(dimethylaminoethylmethacrylate-co-methacrylic esters), poly(ethyl acrylate-co-methyl methacrylate-co-trimethylammonioethyl methacrylate chloride), poly(methyl acrylate-co-methyl methacrylate-co-methacrylic acid) 7:3:1, poly(methacrylic acid-co-methylmethacrylate) 1:2, poly(methacylic acid-co-ethyl acrylate) 1:1, poly(methacylic acid-co-methyl methacrylate) 1:1, polyethylene oxide (PEO), polyethylene glycol (PEG), polyvinyl caprolactam-polyvinyl acetate-polyethylene glycol graft copolymer 57/30/13, polyethylene glycol-polyvinyl alcohol graft copolymer 25/75, Kollicoat IR-polyvinyl alcohol 60/40, polyvinyl alcohol (PVA), amino alkyl methacrylate copolymer E, hydroxypropyl methylcellulose acetate succinate or hypromellose acetate succinate (HPMCAS), methacrylic ester copolymer, glycerol, and ammonioalkyl methacrylate copolymer.

In some embodiments, the immediate release erodible thermoplastic material comprises any one or more of copolyvidone, polyvinylpyrrolidone-co-vinyl-acetate (PVP-VA), polyvinylpyrrolidone-polyvinyl acetate copolymer (PVP-VA) 60/40, polyvinylpyrrolidone (PVP), hydroxypropyl cellulose (HPC), hydroxylpropyl methylcellulose or Hypromellose (HPMC), hydroxypropyl methylcellulose phthalate (HPMCP), methyl cellulose (MC), methacrylic acid copolymer, poly(butyl methacrylate-co-(2-dimethylaminoethyl) methacrylate-co-methyl methacrylate) 1:2:1, poly(dimethylaminoethylmethacrylate-co-methacrylic esters), poly(ethyl acrylate-co-methyl methacrylate-co-trimethylammonioethyl methacrylate chloride), poly(methyl acrylate-co-methyl methacrylate-co-methacrylic acid) 7:3:1, poly(methacrylic acid-co-methylmethacrylate) 1:2, poly(methacylic acid-co-ethyl acrylate) 1:1, poly(methacylic acid-co-methyl methacrylate) 1:1, polyethylene oxide (PEO), polyethylene glycol (PEG), polyvinyl caprolactam-polyvinyl acetate-polyethylene glycol graft copolymer 57/30/13, polyethylene glycol-polyvinyl alcohol graft copolymer 25/75, Kollicoat IR-polyvinyl alcohol 60/40, polyvinyl alcohol (PVA), aminoalkyl methacrylate copolymer E, hydroxypropyl methylcellulose acetate succinate or hypromellose acetate succinate (HPMCAS), methacrylic ester copolymer, ammonioalkyl methacrylate copolymer, ethyl cellulose (EC), polyvinyl acetate (PVAc) and polyvinylpyrrolidone (PVP) 80/20, polyvinyl acetal diethyl aminolactate, and polyvinyl acetal diethyl amino lactate (AEA).

In some embodiments, the drug-diffusion material comprises a swellable polymer impregnated with a drug, e.g., such that upon swelling the drug is release from the drug-diffusion material. In some embodiments, the drug-diffusion material comprises any one or more of cellulose acetate phthalate (CAP), ammonio methacrylate copolymer, poly(lactide-co-glycolide) (PLGA), ethylene-vinyl acetate copolymer, polyethylene (PE), polycaprolactone (PCL), polylactic acid (PLA), ellulose acetate butyrate (CAB), cellulose acetate (CA), polyvinyl acetate (PVAc), polyvinyl acetal diethyl amino lactate (AEA), poly(butyl methacrylate-co-(2-dimethylaminoethyl) methacrylate-co-methyl methacrylate) 1:2:1, ethyl cellulose (EC), polyvinyl acetate (PVAc), polyvinylpyrrolidone (PVP) 80/20, and crospovidone.

In some embodiments, the plasticizer comprises any one or more of triethyl citrate (TEC), vitamin e polyethylene glycol succinate (TPGS), acetin, acetylated triethyl citrate, tributyl citrate, tributyl o-acetylcitrate, polyoxyl 15 hydroxystearate, peg-40 hydrogenated castor oil, polyoxyl 35 castor oil, dibutyl sebacate, diethylphthalate, glycerine, methyl 4-hydroxybenzoate, castor oil, oleic acid, triacetin, polyalkylene glycol.

In some embodiments, the other additive comprises any one or more of acacia, alginate, alginic acid, aluminum acetate, butylparaben, butylated hydroxytoluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid (EDTA), calcium hydrogen phosphate dehydrate, cetylpyridine chloride, calcium phosphate dibasic, calcium phosphate tribasic, dibasic calcium phosphate, disodium hydrogen phosphate, dimethicone, erythrosine sodium, ethylenediaminetetraacetic acid (EDTA), gelatin, glyceryl monooleate, iron oxide, ferric oxide, iron oxide yellow, iron oxide red, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sodium metabisulfite, sodium citrate dehydrate, sodium starch, sodium carboxy methyl cellulose, succinic acid, sodium propionate, titanium dioxide, talc.

In some embodiments, the sustained-release oral drug dosage form comprises one or more of hydroxypropyl cellulose (HPC EF), vinylpyrrolidone-vinyl acetate copolymer (e.g., VA64 or copovidone), triethyl citrate (TEC), and glycerin. In some embodiments, the sustained-release oral drug dosage form comprises HPC EF at about 35 w/w % to about 45 w/w %, VA64 at about 5 w/w % to about 15 w/w %, and glycerin at about 10 w/w % to about 20 w/w %.

ii. Delay Components

The delayed sustained-release oral drug dosage forms described herein comprise a delay component configured to prevent and/or inhibit the release of a JAK inhibitor from the oral drug dosage form for a desired amount of time after administration of the delayed sustained-release oral drug dosage form to a human individual.

In some embodiments, the delay component does not contain a JAK inhibitor.

In some embodiments, the delay component surrounds the sustained-release drug component. In some embodiments, the delay component completely surrounds the sustained-release drug component.

In some embodiments, the delay component comprises an erodible material. In some embodiments, the erodible material of the delay component is different than the erodible material of the sustained-release drug component. In some embodiments, the delay component comprises: a delay member comprising a second erodible material not admixed with a JAK inhibitor; and a shell. In some embodiments, the delay member and the shell surround, such as completely surround, the sustained-release drug component.

iii. Delay Members

The delay members described herein comprise an erodible material not admixed with a JAK inhibitor. The delay members may be formed using numerous materials having varying shapes and sizes. In some embodiments, the delay member is a layer.

In some embodiments, the delay members are configured having surfaces, such as a surface exposed to a bodily fluid during administration of the oral drug dosage form to a human individual, having a pre-determined shape and surface area. For example, in some embodiments, the delay member has a top surface and a bottom surface, wherein the top surface is exposed to a bodily fluid prior to the bottom surface. In some embodiments, the delay member is a layer having a top surface and a bottom surface. In some embodiments, the top surface of the delay member is not flat, e.g., comprises certain features that extend beyond a top surface plane or surface tolerance threshold (as measured between two parallel planes), such as to reduce adherence of the delay member, or a portion thereof, to an internal body part of the human individual. In some embodiments, the top surface of the delay member, or at least a portion thereof, is flat or within a surface tolerance threshold.

The top surface of the delay member, as based on the surface exposed to a bodily fluid, can have any shape. In some embodiments, the top surface of the delay member, as based on the surface exposed to a bodily fluid, has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the top surface of the delay member, such as a delay member layer, has a surface area of about 10 mm² to about 400 mm², such as any of about 20 mm² to about 200 mm², about 20 mm² to about 100 mm², about 20 mm² to about 60 mm², about 30 mm² to about 50 mm². In some embodiments, the top surface of the delay member has a surface area of at least about 20 mm², such as at least about any of 22 mm², 24 mm², 26 mm², 28 mm², 30 mm², 32 mm², 33 mm², 34 mm², 36 mm², 38 mm², 40 mm², 42 mm², 44 mm², 46 mm², 48 mm², 50 mm², 52 mm², 54 mm², 56 mm², 58 mm², 60 mm², 65 mm², 70 mm², 80 mm², 85 mm², 90 mm², 95 mm², 100 mm, 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm², or 400 mm². In some embodiments, the top surface of the delay member has a surface area of less than about 400 mm², such as less than about any of 400 mm², 375 mm², 350 mm², 325 mm², 300 mm², 275 mm², 250 mm², 225 mm², 200 mm², 190 mm², 180 mm², 170 mm², 160 mm², 150 mm², 140 mm², 130 mm², 120 mm², 110 mm², 100 mm², 95 mm², 90 mm², 85 mm², 80 mm², 75 mm², 70 mm², 65 mm², 60 mm², 58 mm², 56 mm², 54 mm², 52 mm², 50 mm², 48 mm², 46 mm², 44 mm², 42 mm², 40 mm², 38 mm², 36 mm², 34 mm², 32 mm², 30 mm², 28 mm², 26 mm², 24 mm², 22 mm², or 20 mm². In some embodiments, the top surface of the delay member has a surface area of about any of 20 mm², 21 mm², 22 mm², 23 mm², 24 mm², 25 mm², 26 mm², 27 mm², 28 mm, 29 mm², 30 mm², 31 mm², 32 mm², 33 mm², 34 mm², 35 mm², 36 mm², 37 mm², 38 mm², 39 mm², 40 mm², 41 mm², 42 mm², 43 mm 2, 44 mm², 45 mm², 46 mm², 47 mm², 48 mm², 49 mm², 50 mm^{,} 51 mm², 52 mm², 53 mm², 54 mm², 55 mm², 56 mm², 57 mm², 58 mm², 59 mm², 60 mm², 65 mm², 70 mm², 80 mm², 85 mm², 90 mm 2, 95 mm², 100 mm², 110 mm², 120 mm², 130 mm², 140 mm², 150 mm², 160 mm², 170 mm², 180 mm², 190 mm², 200 mm², 225 mm², 250 mm², 275 mm², 300 mm², 325 mm², 350 mm², 375 mm 2, or 400 mm².

In some embodiments, the surface area of the top surface of the delay member exposed to a bodily fluid is consistent throughout the thickness of the delay member, e.g., as the delay member erodes the surface exposed to the bodily fluid has the same surface area. In some embodiments, the surface area of the top surface of the delay member exposed to a bodily fluid is different at two or more points, e.g., as the delay member erodes the surface exposed to the bodily fluid changes such as increases and/or decreases in surface area during erosion of the delay member. In some embodiments, the shape of the surface of the delay member exposed to a bodily fluid is consistent throughout the thickness of the delay member, e.g., as the delay member erodes the surface exposed to the bodily fluid is the same shape. In some embodiments, the shape of the surface of the delay member exposed to a bodily fluid is different at two or more points. In some embodiments, the bottom surface of the delay member has a surface area that is the same as that of the top surface of the delay member. In some embodiments, the bottom surface of the delay member has a surface area that is different than that of the top surface of the delay member.

In some embodiments, the top surface of the delay member, such as a delay member layer, has a largest crossing dimension of about 5 mm to about 20 mm, such as any of about 5 mm to about 15 mm, about 6 mm to about 13 mm, or about 7 to about 11 mm. In some embodiments, the top surface of the delay member has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the top surface of the delay member has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the top surface of the delay member has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the top surface of the delay member, such as a delay member layer, has a crossing dimension perpendicular to the largest crossing dimension of about 1 mm to about 15 mm, such as any of about 2 mm to about 10 mm, about 2 mm to about 6 mm, or about 1 to about 5 mm. In some embodiments, the top surface of the delay member has a crossing dimension perpendicular to the largest crossing dimension of at least about 1 mm, such as at least about any of 2 mm, 3 mm, 4 mm, 5 mm, 6 mm, 7 mm, 8 mm, 9 mm, 10 mm, 11 mm, 12 mm, 13 mm, 14 mm, or 15 mm. In some embodiments, the top surface of the delay member has a crossing dimension perpendicular to the largest crossing dimension of less than about 15 mm, such as less than about any of 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the top surface of the delay member has a crossing dimension perpendicular to the largest crossing dimension of about any of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm.

In some embodiments, the delay member, such as a delay member layer, has a thickness of about 0.1 mm to about 5 mm, such as any of about 0.2 mm to about 2 mm, about 0.5 mm to about 1.5 mm, or about 0.8 mm to about 1.4 mm. In some embodiments, the delay member has a thickness of at least about 0.1 mm, such as at least about any of 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm. In some embodiments, the delay member has a thickness of less than about 5 mm, such as less than about any of 4.8 mm, 4.6 mm, 4.4 mm, 4.2 mm, 4.0 mm, 3.8 mm, 3.6 mm, 3.4 mm, 3.2 mm, 3.0 mm, 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, 0.4 mm, 0.3 mm, 0.2 mm, or 0.1 mm. In some embodiments, the delay member has a thickness of about any of 0.1 mm, 0.2 mm, 0.3 mm, 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5 mm.

In some embodiments, the delay member, such as a delay member layer, comprises a top surface and a bottom surface, wherein the thickness, as measured between the top surface and the bottom surface, is substantially consistent, such as within a 20% range of an average thickness.

In some embodiments, the delay member, such as a delay member layer, comprises a side surface.

In some embodiments, the delay member comprises an erodible material not admixed with the JAK inhibitor. In some embodiments, the delay member, such as the erodible material of the delay member, comprises a thermoplastic material, for example a thermoplastic polymer. In some embodiments, the delay member comprises a material, such as any one or more of an erodible thermoplastic material, a plasticizer, and another additive, e.g., a filler, a binder, a lubricant, a glidant, and a disintegrant.

In some embodiments, the erodible thermoplastic material comprises any one or more of copolyvidone, polyvinylpyrrolidone-co-vinyl-acetate (PVP-VA), polyvinylpyrrolidone-polyvinyl acetate copolymer (PVP-VA) 60/40, polyvinylpyrrolidone (PVP), hydroxypropyl cellulose (HPC), hydroxylpropyl methylcellulose or Hypromellose (HPMC), hydroxypropyl methylcellulose phthalate (HPMCP), methyl cellulose (MC), methacrylic acid copolymer, poly(butyl methacrylate-co-(2-dimethylaminoethyl) methacrylate-co-methyl methacrylate) 1:2:1, poly(dimethylaminoethylmethacrylate-co-methacrylic esters), poly(ethyl acrylate-co-methyl methacrylate-co-trimethylammonioethyl methacrylate chloride), poly(methyl acrylate-co-methyl methacrylate-co-methacrylic acid) 7:3:1, poly(methacrylic acid-co-methylmethacrylate) 1:2, poly(methacylic acid-co-ethyl acrylate) 1:1, poly(methacylic acid-co-methyl methacrylate) 1:1, polyethylene oxide (PEO), polyethylene glycol (PEG), polyvinyl caprolactam-polyvinyl acetate-polyethylene glycol graft copolymer 57/30/13, polyethylene glycol-polyvinyl alcohol graft copolymer 25/75, Kollicoat IR-polyvinyl alcohol 60/40, polyvinyl alcohol (PVA), aminoalkyl methacrylate copolymer E, hydroxypropyl methylcellulose acetate succinate or hypromellose acetate succinate (HPMCAS), methacrylic ester copolymer, ammonioalkyl methacrylate copolymer, ethyl cellulose (EC), polyvinyl acetal diethyl aminolactate, and polyvinyl acetal diethyl amino lactate (AEA).

In some embodiments, the plasticizer comprises any one or more of triethyl citrate (TEC), vitamin E polyethylene glycol succinate (TPGS), aacetin, acetylated triethyl citrate, tributyl citrate, tributyl o-acetylcitrate, polyoxyl 15 hydroxystearate, peg-40 hydrogenated castor oil, polyoxyl 35 castor oil, dibutyl sebacate, diethylphthalate, glycerine, methyl 4-hydroxybenzoate, castor oil, oleic acid, triacetin, and polyalkylene glycol.

In some embodiments, the other additive comprises any one or more of acacia, alginate, alginic acid, aluminum acetate, barium sulfate, butylparaben, butylated hydroxytoluene, citric acid, calcium carbonate, calcium perphosphate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid (EDTA), calcium hydrogen phosphate dehydrate, cetylpyridine chloride, calcium phosphate dibasic, calcium phosphate tribasic, dibasic calcium phosphate, disodium hydrogen phosphate, dimethicone, erythrosine sodium, ethylenediaminetetraacetic acid (EDTA), gelatin, glycerol, glyceryl monooleate, iron oxide, ferric oxide, iron oxide yellow, iron oxide red, L100-55, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sodium metabisulfite, sodium citrate dehydrate, sodium starch, sodium carboxy methyl cellulose, succinic acid, sodium propionate, titanium dioxide, and talc.

In some embodiments, the delay member comprises one or more of hydroxypropyl cellulose (HPC EF), triethyl citrate (TEC), and titanium dioxide. In some embodiments, the delay member comprises HPC EF at about 80 w/w % to about 90 w/w %, TEC at about 10 w/w % to about 20 w/w %, and titanium dioxide at about 0.1 w/w % to about 0.3 w/w %.

iv. Shells

In some embodiments, the delay component comprises a shell. In some embodiments, the shells are configured having surfaces, such as an exterior facing surface exposed to a bodily fluid during administration of the oral drug dosage form to a human individual. In some embodiments, the exterior surface of the shell is not flat, e.g., comprises certain features that extend beyond a surface plane or surface tolerance threshold (as measured between two parallel planes), such as to reduce adherence of the shell, or a portion thereof, to an internal body part of the human individual. In some embodiments, the exterior surface of the shell, or at least a portion thereof, is flat or within a surface tolerance threshold.

The surfaces of the shell, as based on the surfaces exposed to a bodily fluid, can have any shape. In some embodiments, the surface of a shell, as based on the surface exposed to a bodily fluid, has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the shell has a largest crossing dimension of about 5 mm to about 20 mm, such as any of about 5 mm to about 15 mm, about 6 mm to about 13 mm, or about 7 to about 11 mm. In some embodiments, the shell has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the shell has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the shell has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the largest crossing dimension is measured across a surface of the delayed sustained-release oral drug dosage form.

In some embodiments, the shell has a crossing dimension perpendicular to a largest crossing dimension of about 5 mm to about 20 mm, such as any of about 5 mm to about 15 mm, about 6 mm to about 13 mm, or about 7 to about 11 mm. In some embodiments, the shell has a crossing dimension perpendicular to a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the shell has a crossing dimension perpendicular to a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the shell has a crossing dimension perpendicular to a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the crossing dimension perpendicular to a largest crossing dimension is measured across a surface of the delayed sustained-release oral drug dosage form.

In some embodiments, the shell is configured to have a thickness to prevent and/or inhibit exposure of components of the delayed sustained-release oral drug dosage form, or a portion thereof, to a bodily fluid. In some embodiments, the shell has a thickness, as measured from an exterior surface of the delayed sustained-release oral drug dosage form to another component thereof, of about 0.4 mm to about 3 mm, such as any of about 0.4 mm to about 2 mm, or about 0.5 mm to about 1.5 mm. In some embodiments, the shell has a thickness of at least about 0.4 mm, such as at least about any of 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm. In some embodiments, the shell has a thickness of less than about 3 mm, such as less than about any of 2.8 mm, 2.6 mm, 2.4 mm, 2.2 mm, 2.0 mm, 1.9 mm, 1.8 mm, 1.7 mm, 1.6 mm, 1.5 mm, 1.4 mm, 1.3 mm, 1.2 mm, 1.1 mm, 1.0 mm, 0.9 mm, 0.8 mm, 0.7 mm, 0.6 mm, 0.5 mm, or 0.4 mm. In some embodiments, the shell has a thickness of about any of 0.4 mm, 0.5 mm, 0.6 mm, 0.7 mm, 0.8 mm, 0.9 mm, 1.1 mm, 1.2 mm, 1.3 mm, 1.4 mm, 1.5 mm, 1.6 mm, 1.7 mm, 1.8 mm, 1.9 mm, 2.0 mm, 2.2 mm, 2.4 mm, 2.6 mm, 2.8 mm, 3.0 mm.

In some embodiments, the shell comprises a side surface.

In some embodiments, the shell comprises an insulating material that is impermeable to bodily fluids, such as gastrointestinal fluid. In some embodiments, the shell comprises an insulating material that is impermeable to certain bodily fluids, such as stomach fluid. In some embodiments, the shell comprises an insulating material that is impermeable to bodily fluids at a certain pH, e.g., an insulating material that is impermeable to bodily fluids at a pH of less than about 6.

In some embodiments, the shell comprises an insulating material that is a non-erodible material. In some embodiments, the shell comprises an insulating material that is non-erodible in certain bodily fluids, such as stomach fluid. In some embodiments, the shell comprises an insulating material that is non-erodible in bodily fluids at a certain pH, e.g., an insulating material that is impermeable to bodily fluids at a pH of less than about 6. In some embodiments, the insulating material is an enteric material.

In some embodiments, the shell comprises an insulating material that is an erodible material having a pH-sensitive erosion and/or an erosion rate that allows for the complete release of a JAK inhibitor from a delayed sustained-release oral drug dosage form prior to exposure of a sustained-release drug component to bodily fluids due to erosion of the shell.

In some embodiments, the shell comprises an insulating material that is selectively permeable. For example, in some embodiments, the shell is permeable to a bodily fluid, and is impermeable to a compound, such as a JAK inhibitor.

In some embodiments, the shell comprises a thermoplastic material, such as a thermoplastic polymer. In some embodiments, the shell comprises a material, such as any one or more of an insoluble material, a swelling material, a plasticizer, and another additive, e.g., a filler, a binder, a lubricant, a glidant, and a disintegrant.

In some embodiments, the insoluble material is any one or more of cellulose acetate phthalate (CAP), ammonio methacrylate copolymer, poly(lactide-co-glycolide) (PLGA), ethylene-vinyl acetate copolymer, polyethylene (PE), polycaprolactone (PCL), polylactic acid (PLA), ellulose acetate butyrate (CAB), cellulose acetate (CA), polyvinyl acetate (PVAc), polyvinyl acetal diethyl amino lactate (AEA), poly(butyl methacrylate-co-(2-dimethylaminoethyl) methacrylate-co-methyl methacrylate) 1:2:1, and ethyl cellulose (EC).

In some embodiments, the swelling material is any one or more of high molecule weight hydroxypropyl cellulose (HPC) such as HPC of about 700 kDa or greater, high molecular weight hydroxylpropyl methylcellulose or Hypromellose (HPMC) such as HPMC of about 500 kDa or greater, methyl cellulose (MC), high molecular weight polyethylene oxide (PEO) such as PEO of about 700 kDa or greater, high molecular polyvinyl alcohol (PVA) such as PVA of about 150 kDa or greater, polyvinyl acetate (PVAc) and polyvinylpyrrolidone (PVP) 80/20, methacrylic ester copolymer, ammonioalkyl methacrylate copolymer, amino alkyl methacrylate copolymer E, hydroxypropyl methylcellulose acetate succinate or hypromellose acetate succinate (HPMCAS), hydroxypropyl methylcellulose phthalate (HPMCP), and crospovidone.

In some embodiments, the plasticizer is any one or more of triethyl citrate (TEC), vitamin E polyethylene glycol succinate (TPGS), aacetin, acetylated triethyl citrate, tributyl citrate, tributyl o-acetylcitrate, polyoxyl 15 hydroxystearate, PEG-40 hydrogenated castor oil, polyoxyl 35 castor oil, dibutyl sebacate, diethylphthalate, glycerine, methyl 4-hydroxybenzoate, castor oil, oleic acid, triacetin, and polyalkylene glycol.

In some embodiments, the other additive is any one or more of acacia, alginate, alginic acid, aluminum acetate, butylparaben, butylated hydroxytoluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium disodium ethylenediaminetetraacetic acid (EDTA), calcium hydrogen phosphate dehydrate, cetylpyridine chloride, calcium phosphate dibasic, calcium phosphate tribasic, dibasic calcium phosphate, disodium hydrogen phosphate, dimethicone, erythrosine sodium, ethylenediaminetetraacetic acid (EDTA), gelatin, glyceryl monooleate, iron oxide, ferric oxide, iron oxide yellow, iron oxide red, lactose (hydrous, anhydrous, monohydrate, or spray dried), microcrystalline cellulose, magnesium carbonate, magnesium oxide, methyl paraben, polysorbate 80, propylene paraben, potassium bicarbonate, potassium sorbate, potato starch, phosphoric acid, polyoxyethylene (40) stearate, sodium starch glycolate, starch pregelatinized, and sodium crossmellose, sodium lauryl sulfate, starch, silicon dioxide, sodium benzoate, sucrose, sorbic acid, sodium carbonate, saccharin sodium, sodium alginate, silica gel, sorbitan monooleate, sodium chloride, sodium metabisulfite, sodium citrate dehydrate, sodium starch, sodium carboxy methyl cellulose, succinic acid, sodium propionate, titanium dioxide, talc.

In some embodiments, the shell comprises one or more of ammonio methacrylate copolymer type B, ethylcellulose, stearic acid, and titanium dioxide. In some embodiments, the shell comprises ammonio methacrylate copolymer type B at about 60 w/w % to about 70 w/w %, ethylcellulose at about 10 w/w % to about 20 w/w %, stearic acid at about 15 w/wo % to about 25 w/w %, and titanium dioxide at about 0.1 w/w/% to about 0.3 w/w %.

v. Janus Kinase (JAK) Inhibitors

In some embodiments, the JAK inhibitor is an agent that interferes with the JAK-STAT signaling pathway, such an inhibitor of one or more members associated with the JAK-STAT signaling pathway (e.g., a JAKinib). Members associated with the JAK-STAT signaling pathway, and inhibitors thereof, are known in the art. See, e.g., Rawlings et al., J Cell Sci, 117, 2004; and Schwartz et al, Nat Rev Drug Discov, 17, 2017.

In some embodiments, the JAK inhibitor is an inhibitor of any one or more of Janus kinase 1 (JAK1), Janus kinase 2 (JAK2), Janus kinase 3 (JAK3), or tyrosine kinase 2 (TYK2). In some embodiments, the JAK inhibitor is an inhibitor of JAK1 and JAK 3. In some embodiments, the JAK inhibitor is an inhibitor of JAK1, JAK3, and JAK2. In some embodiments, the JAK inhibitor is an inhibitor of JAK1, JAK3, JAK2, and TYK2. In some embodiments, the JAK inhibitor is an inhibitor of JAK1 and JAK2. In some embodiments, the JAK inhibitor is an inhibitor of JAK1, JAK2, and TYK2. In some embodiments, the JAK inhibitor is an inhibitor of all JAKs (a pan-JAK inhibitor).

In some embodiments, the JAK inhibitor is selected from the group consisting of tofacitinib, abrocitinb, baricitinib, cerdulatinib, cucurbitacin I, decernotinib, fedratinib, filgotinib, gandotinib, itacitinib, lestaurtinib, momelotinib, oclacitinib, pacritinib, peficitinib, ruxolitinib, solcitinib, upadacitinib, BMS-986165, CHZ868, and SHR0302, or a pharmaceutically acceptable salt thereof. In some embodiments, the delayed sustained-release oral drug dosage form comprises a plurality of JAK inhibitors, wherein each JAK inhibitor is selected from the group consisting of tofacitinib, abrocitinb, baricitinib, cerdulatinib, cucurbitacin I, decernotinib, fedratinib, filgotinib, gandotinib, itacitinib, lestaurtinib, momelotinib, oclacitinib, pacritinib, peficitinib, ruxolitinib, solcitinib, upadacitinib, BMS-986165, CHZ868, and SHR0302, or a pharmaceutically acceptable salt thereof.

In some embodiments, the JAK inhibitor is tofacitinib or a pharmaceutically acceptable salt thereof. In some embodiments, the JAK inhibitor is tofacitinib citrate, such as tofacitinib mono-citrate. In some embodiments, the JAK inhibitor is tofacitinib tartrate, such as tofacitinib mono-tartrate. In some embodiments, the JAK inhibitor is tofacitinib malate, such as tofacitinib mono-malate. In some embodiments, the JAK inhibitor is tofacitinib oxalate such as tofacitinib mono-oxalate.

In some embodiments, the JAK inhibitor is a pharmaceutically acceptable salt in an amorphous form. In some embodiments, the JAK inhibitor is a pharmaceutically acceptable salt in a crystalline form.

In some embodiments, the amount of the JAK inhibitor in the delayed sustained-release oral drug dosage form is about 1 mg to about 50 mg, such as any of about 1 mg to about 25 mg, about 10 mg to about 40 mg, about 10 mg to about 30 mg, about 9 mg to about 12 mg, about 10 mg to about 12 mg, about 19 mg to about 23 mg, or about 21 mg to about 23 mg. In some embodiments, the amount of the JAK inhibitor in the delayed sustained-release oral drug dosage form is about 1 mg or more, such as about any of 3 mg or more, 4 mg or more, 5 mg or more, 6 mg or more, 7 mg or more, 8 mg or more, 9 mg or more, 10 mg or more, 11 mg or more, 12 mg or more, 13 mg or more, 14 mg or more, 15 mg or more, 16 mg or more, 17 mg or more, 18 mg or more, 19 mg or more, 20 mg or more, 21 mg or more, 22 mg or more, 23 mg or more, 24 mg or more, or 25 mg or more. In some embodiments, the amount of the JAK inhibitor in the delayed sustained-release oral drug dosage form is about 25 mg or less, such as about any of 24 mg or less, 23 mg or less, 22 mg or less, 21 mg or less, 20 mg or less, 19 mg or less, 18 mg or less, 17 mg or less, 16 mg or less, 15 mg or less, 14 mg or less, 13 mg or less, 12 mg or less, 11 mg or less, 10 mg or less, 9 mg or less, 8 mg or less, 7 mg or less, 6 mg or less, 5 mg or less, 4 mg or less, or 3 mg or less. In some embodiments, the amount of the JAK inhibitor in the delayed sustained-release oral drug dosage form is about any of 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

In some embodiments, the amount of the JAK inhibitor in the delayed sustained-release oral drug dosage form is about 11 mg.

In some embodiments, the amount of the JAK inhibitor in the delayed sustained-release oral drug dosage form is about 22 mg.

vi. Configurations of Components of the Sustained-Release Drug Components and Delay Components

The components described herein can be configured in various fashions to form the disclosed delayed sustained-release oral drug dosage forms.

In some embodiments, wherein the delayed sustained-release oral drug dosage form comprises a sustained-release drug component (such as a sustained-release drug layer) and a delay component comprising a delay member (such as a delay member layer) and a shell, the sustained-release drug component and the delay component are embedded in the shell. In some embodiments, at least a portion of the sustained-release drug component is in direct contact with the shell. In some embodiments, the sustained-release drug component comprises a bottom surface, wherein at least a portion of the bottom surface of the sustained-release drug component is in direct contact with the shell. In some embodiments, the sustained-release drug component comprises a side surface, wherein at least a portion of the side surface is in direct contact with the shell. In some embodiments, the sustained-release drug component comprises a bottom surface and a side surface, wherein at least a portion of the bottom surface and the side surface of the sustained-release drug component are in direct contact with the shell. In some embodiments, the sustained-release drug component comprises a top surface, wherein at least a portion of the top surface is not in direct contact with the shell. In some embodiments, at least a portion of the delay member is in direct contact with the shell. In some embodiments, the delay member comprises a bottom surface, wherein at least a portion of the bottom surface of the delay member is in direct contact with the shell. In some embodiments, the delay member comprises a bottom surface, wherein the bottom surface of the delay member is not in direct contact with the shell. In some embodiments, the delay member comprises a side surface, wherein at least a portion of the side surface of the delay member is in direct contact with the shell. In some embodiments, the delay member comprises a side surface, wherein the side surface of the delay member is not in direct contact with the shell. In some embodiments, the delay member comprises a bottom surface and a side surface, wherein at least a portion of the bottom surface and the side surface of the delay member are in direct contact with the shell. In some embodiments, the delay member comprises a top surface, wherein at least a portion of the top surface of the delay member is not in direct contact with the shell. In some embodiments, at least a portion of the top surface of the sustained-release drug component is in direct contact with at least a portion of the bottom surface of the delay member. In some embodiments, the surface area of the top surface of the sustained-release drug component is the same as the surface area of the bottom surface of the delay member. In some embodiments, the surface area of the top surface of the sustained-release drug component is less than the surface area of the bottom surface of the delay member.

In some embodiments, the delay member and the shell are configured such that the JAK inhibitor is prevented from being released from the delayed sustained-release oral drug dosage form until after the delay member is eroded. In some embodiments, less than about 5%, such as less than about any of 4%, 3%, 2%, or 1%, of the JAK inhibitor in the oral drug dosage form is release from the oral drug dosage form within about 2 hours after administration of the oral drug dosage form to a human individual.

For purposes of illustration, exemplary configurations of delayed sustained-release oral drug dosage forms comprising: a sustained-release drug component comprising an erodible material admixed with a JAK inhibitor; and a delay component comprising: a delay member comprising an erodible material not admixed with the JAK inhibitor; and a shell, are described below.

As shown in FIG. 1A, in some embodiments, the sustained-release drug component, the delay member, and the shell are configured such that: the sustained-release drug component is embedded in the shell; the delay member is embedded in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; a portion of the bottom surface of the delay member is in direct contact with the shell; and the side surface of the delay member is in direct contact with the shell. The portion of the bottom surface of the delay member that is in direct contact with the shell forms a perimeter extending beyond the top surface of the sustained-release drug component. The shell has an inset for both the sustained-release drug component and the delay member such that the components are embedded in the shell. The top surface of the delayed sustained-release oral drug dosage form (the surface from which the JAK inhibitor will be released from the oral drug dosage form) is formed from the shell and the delay member. In some embodiments, the shell and the delay member are configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

As shown in FIG. 1C, in some embodiments, the sustained-release drug component, the delay member, and the shell are configured such that: the sustained-release drug component is embedded in the shell; the delay member is embedded in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; and the side surface of the delay member is in direct contact with the shell. Optionally, a portion of the bottom surface of the delay member may be in direct contact with the shell. In such embodiments, the portion of the bottom surface of the delay member that is in direct contact with the shell forms a perimeter extending beyond the top surface of the sustained-release drug component. The shell has an inset for both the sustained-release drug component and the delay member such that the components are embedded in the shell. The top surface of the delayed sustained-release oral drug dosage form (the surface from which the JAK inhibitor will be released from the oral drug dosage form) is formed from the shell and the delay member. In some embodiments, the shell and the delay member are configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

As shown in FIG. 1D, in some embodiments, the sustained-release drug component, the delay member, and the shell are configured such that: the sustained-release drug component is embedded in the shell; the delay member is embedded in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; a portion of the bottom surface of the delay member is in direct contact with the shell; and the side surface of the delay member is in direct contact with the shell. The portion of the bottom surface of the delay member that is in direct contact with the shell forms a perimeter extending beyond the top surface of the sustained-release drug component. The shell has an inset for both the sustained-release drug component and the delay member such that the components are embedded in the shell. The top surface of the delayed sustained-release oral drug dosage form (the surface from which the JAK inhibitor will be released from the oral drug dosage form) is formed from the shell. In some embodiments, the shell is configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

As shown in FIG. 1E, in some embodiments, the sustained-release drug component, the delay member, and the shell are configured such that: the sustained-release drug component is embedded in the shell; the delay member is embedded, in part, in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; a portion of the bottom surface of the delay member is in direct contact with the shell; a portion of the side surface of the delay member is in direct contact with the shell; a portion of the side surface of the delay member is not in direct contact with the shell. The portion of the bottom surface of the delay member that is in direct contact with the shell forms a perimeter extending beyond the top surface of the sustained-release drug component. The shell has an inset for both the sustained-release drug component and the delay member such that the sustained-release drug component is embedded in the shell and the delay member is, in part, embedded in the shell. The top surface of the delayed sustained-release oral drug dosage form (the surface from which the JAK inhibitor will be released from the oral drug dosage form) is formed from the delay member. In some embodiments, the shell and the delay member are configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

As shown in FIG. 1F, in some embodiments, the sustained-release drug component, the delay member, and the shell are configured such that: the sustained-release drug component is embedded in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; a portion of the bottom surface of the delay member is in direct contact with the shell; and the side surface of the delay member is not in direct contact with the shell. The portion of the bottom surface of the delay member that is in direct contact with the shell forms a perimeter extending beyond the top surface of the sustained-release drug component. The shell has an inset for the sustained-release drug component such that the sustained-release drug component is embedded in the shell. The top surface of the delayed sustained-release oral drug dosage form (the surface from which the JAK inhibitor will be released from the oral drug dosage form) is formed from the delay member. In some embodiments, the shell and the delay member are configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

As shown in FIG. 1G, in some embodiments, the sustained-release drug component, the delay member, and the shell are configured such that: the sustained-release drug component is embedded in the shell; the delay member is embedded in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; and the side surface of the delay member is in direct contact with the shell. The top surface of the sustained-release drug component has the same surface area as the surface area of the bottom surface of the delay member. The shell has an inset for both the sustained-release drug component and the delay member such that the components are embedded in the shell. The top surface of the delayed sustained-release oral drug dosage form (the surface from which the JAK inhibitor will be released from the oral drug dosage form) is formed from the shell. In some embodiments, the shell is configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

As shown in FIG. 1H, in some embodiments, the sustained-release drug component, the delay member, and the shell are configured such that: the sustained-release drug component is embedded in the shell; the delay member is embedded in the shell; the bottom surface of the sustained-release drug component is in direct contact with the shell; the side surface of the sustained-release drug component is in direct contact with the shell; the top surface of the sustained-release drug component is in direct contact with the bottom surface of the delay member; and the side surface of the delay member is in direct contact with the shell. The top surface of the sustained-release drug component has the same surface area as the surface area of the bottom surface of the delay member. The shell has an inset for both the sustained-release drug component and the delay member such that the components are embedded in the shell. The top surface of the delayed sustained-release oral drug dosage form (the surface from which the JAK inhibitor will be released from the oral drug dosage form) is formed from the shell and the delay member. In some embodiments, the shell and the delay member are configured to facilitate release of the JAK inhibitor from the delayed sustained-release oral drug dosage form e.g., by reducing adherence of a portion of the oral drug dosage form to an internal body part of the human individual that may prevent or inhibit, to any degree, the release of the JAK inhibitor from the oral drug dosage.

The delayed sustained-release oral drug dosage forms described herein are suitable for oral administration to a human individual. The drug dosage forms of the present invention can be, for example, any size, shape, or weight that is suitable for oral administration to specific human individuals, such as children and adults. In some embodiments, the drug dosage form is suitable for oral administration to an individual, wherein selection of size, shape, or weight of the drug dosage form is based on an attribute of the individual, e.g., one or more of height, weight, or age.

In some embodiments, a surface of the delayed sustained-release oral drug dosage form has the shape of a capsule, circle, oval, bullet shape, arrow head shape, triangle, arced triangle, square, arced square, rectangle, arced rectangle, diamond, pentagon, hexagon, octagon, half moon, almond, or a combination thereof.

In some embodiments, the delayed sustained-release oral drug dosage form has a largest crossing dimension of about 5 mm to about 20 mm, such as any of about 5 mm to about 15 mm, about 6 mm to about 13 mm, or about 7 to about 11 mm. In some embodiments, the delayed sustained-release oral drug dosage form has a largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the delayed sustained-release oral drug dosage form has a largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the delayed sustained-release oral drug dosage form has a largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the largest crossing dimension is measured across a surface of the delayed sustained-release oral drug dosage form.

In some embodiments, the delayed sustained-release oral drug dosage form has a crossing dimension perpendicular to the largest crossing dimension of about 5 mm to about 20 mm, such as any of about 5 mm to about 15 mm, about 6 mm to about 13 mm, or about 7 to about 11 mm. In some embodiments, the delayed sustained-release oral drug dosage form has a crossing dimension perpendicular to the largest crossing dimension of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the delayed sustained-release oral drug dosage form has a crossing dimension perpendicular to the largest crossing dimension of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the delayed sustained-release oral drug dosage form has a crossing dimension perpendicular to the largest crossing dimension of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the crossing dimension perpendicular to a largest crossing dimension is measured across a surface of the delayed sustained-release oral drug dosage form.

In some embodiments, the delayed sustained-release oral drug dosage form has a thickness of about 5 mm to about 20 mm, such as any of about 5 mm to about 15 mm, about 6 mm to about 13 mm, or about 7 to about 11 mm. In some embodiments, the delayed sustained-release oral drug dosage form has a thickness of at least about 5 mm, such as at least about any of 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. In some embodiments, the delayed sustained-release oral drug dosage form has a thickness of less than about 20 mm, such as less than about any of 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In some embodiments, the delayed sustained-release oral drug dosage form has a thickness of about any of 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

In some embodiments, the delayed sustained-release oral drug dosage form has a total weight of about 50 mg to about 1,000 mg, such as any of about 50 mg to about 100 mg, about 100 to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, or about 900 mg to about 1,000 mg. In some embodiments, the delayed sustained-release oral drug dosage form has a total weight of at least about 50 mg, such as at least about any of 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1,000 mg. In some embodiments, the delayed sustained-release oral drug dosage form has a total weight of less than about 1,000 mg, such as less than about an of 950 mg, 900 mg, 850 mg, 800 mg, 750 mg, 700 mg, 650 mg, 600 mg, 550 mg, 500 mg, 475 mg, 450 mg, 425 mg, 400 mg, 375 mg, 350 mg, 325 mg, 300 mg, 275 mg, 250 mg, 225 mg, 200 mg, 175 mg, 150 mg, 125 mg, 100 mg, 75 mg, or 50 mg. In some embodiments, the delayed sustained-release oral drug dosage form has a total weight of about any of 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1,000 mg.

In some embodiments, the surface of the delay sustained-release oral drug dosage form from which at least a portion of the JAK inhibitor is configured to be released from is configured to reduce adherence of the oral drug dosage form, or a portion thereof, to an internal body part of the human individual. In some embodiments, the surface, or at least a portion thereof, from which at least a portion of the JAK inhibitor is configured to be released from is not flat, e.g., exceeds a surface tolerance threshold. In some embodiments, the surface, or at least a portion thereof, from which at least a portion of the JAK inhibitor is configured to be released from is flat, e.g., falls within a surface tolerance threshold.

In some embodiments, the delayed sustained-release oral drug dosage form is not an osmotic dosage form, such as an osmotic-controlled release oral drug dosage form.

In some embodiments, the delayed sustained-release oral drug dosage form comprises two or more dosage units, each dosage unit comprising a sustained-release drug component and a delay component, at least in part. For example, in some embodiments, the delayed sustained-release oral drug form comprises a shell comprising a first sustained-release drug component and a second sustained-release drug component embedded therein, and a first delay member and a second delay member, wherein the first delay member prevents the release of the JAK inhibitor from the first sustained-release drug component for about 2 hours to about 6 hours after administration of the delayed sustained-release oral drug dosage form to a human individual, and wherein the second delay member prevents the release of the JAK inhibitor from the first sustained-release drug component for about 2 hours to about 6 hours after administration of the delayed sustained-release oral drug dosage form to a human individual. In some embodiments, the two dosage units are the same. In some embodiments, the two dosage units are different. In some embodiments, the two dosage units are stacked back-to-back. In some embodiments, the two dosage units are separated by the shell.

vii. Additional Components

In some embodiments, the delayed sustained-release dosage form comprises an additional component, such as an outer coating. In some embodiments, the outer coating is a flavor coating. In some embodiments, the outer coating is a sugar coating. In some embodiments, the outer coating is a cosmetic coating. In some embodiments, the outer coating is a color coating. In some embodiments, the outer coating is a film coating. In some embodiments, the outer coating is a polymer coating. In some embodiments, the additional component is a label, such as a company name, abbreviation, or logo, a medication label or drug name, such as a drug brand name and/or drug chemical name or abbreviation, a drug amount or strength, an identification barcode, or any combination thereof.

### B. Release Profiles of Delayed Sustained-Release Oral Drug Dosage Forms

The delayed sustained-release oral drug dosage form described herein are formulated and configured to delay the release of a JAK inhibitor from the oral drug dosage form for a desired amount of time after administration of the oral drug dosage form to a human individual, and following that delay to then release the JAK inhibitor from the oral drug dosage form according to a desired release profile. In some embodiments, the delayed sustained-release oral drug dosage form comprises an immediate release component, such as an immediate release component comprising the JAK inhibitor. In some embodiments, immediate release component releases the JAK inhibitor after an initial delay (i.e., the oral drug dosage form is configured to delay the immediate release component from releasing the drug contained therein from the oral drug dosage form for a desired period of time).

In some embodiments, the JAK inhibitor is prevented and/or inhibited from being release from a delayed sustained-release oral drug dosage form for about 1 hour to about 7 hours, such as any of about 1 hour to about 6 hours, about 2 hours to about 6 hours, about 2 hours to about 4 hours, about 2 hours to about 3 hours, or about 1.5 hours to about 3 hours, after administration of the delayed sustained-release oral drug dosage form to a human individual. In some embodiments, the JAK inhibitor is prevented and/or inhibited from being release from a delayed sustained-release oral drug dosage form for at least about 1 hour, such as at least about any of 1.5 hours, 2 hours, 2.5 hours, 3 hours, 3.5 hours, 4 hours, 4.5 hours, 5 hours, 5.5 hours, 6 hours, or 6.5 hours, but no longer than about 7 hours, after administration of the delayed sustained-release oral drug dosage form to a human individual. In some embodiments, the JAK inhibitor is prevented and/or inhibited from being release from a delayed sustained-release oral drug dosage form for no greater than about 7 hours, such as no great than about any of 6.5 hours, 6 hours, 5.5 hours, 5 hours, 4.5 hours, 4 hours, 3.5 hours, 3 hours, 2.5 hours, 2 hours, 1.5 hours, or 1 hour, after administration of the delayed sustained-release oral drug dosage form to a human individual. In some embodiments, the JAK inhibitor is prevented and/or inhibited from being release from a delayed sustained-release oral drug dosage form for at least about any of 1 hour, 1.5 hours, 2 hours, 2.5 hours, 3 hours, 3.5 hours, 4 hours, 4.5 hours, 5 hours, 5.5 hours, 6 hours, 6.5 hours, or 7 hours, after administration of the delayed sustained-release oral drug dosage form to a human individual.

Following the delay in release of the JAK inhibitor, the oral drug dosage forms described herein are configured to then release the JAK inhibitor according to a desired release profile. As known in the art, efficacy of tofacitinib is observed at plasma concentrations of about 17 ng/mL and above. See, e.g., Meyer et al., J Inflammation, 7, 2010. In some embodiments, when the delayed sustained-release oral drug dosage form is administered to the human individual, the mean area under the plasma concentration versus time curve after complete erosion of the delay component or a portion thereof, such as the delay member, is about 17 ng-hr/mL per mg JAK inhibitor dosed to about 42 ng-hr/mL per mg of JAK inhibitor dosed. In some embodiments, when the delayed sustained-release oral drug dosage form is administered to the human individual, the mean area under the plasma concentration versus time curve after complete erosion of the delay component or a portion thereof, such as the delay member, is above about 17 ng-hr/mL per mg JAK inhibitor dosed for about 6 hours to about 15 hours over a 24 hour period. In some embodiments, when the delayed sustained-release oral drug dosage form is administered to the human individual, the mean area under the plasma concentration versus time curve after complete erosion of the delay component or a portion thereof, such as the delay member, is below about 17 ng-hr/mL per mg JAK inhibitor dosed for about 9 hours to about 18 hours over a 24 hour period. In some embodiments, the delayed sustained-release oral drug dosage form comprises an adequate drug holiday to avoid a decrease in efficacy.

In some embodiments, the delayed sustained-release oral drug dosage form is configured to release the JAK inhibitor according to the following: (i) not more than about 20-40% of the total JAK inhibitor is released at 1 hour after complete erosion of the delay component or a portion thereof, such as the delay member; (ii) not less than about 25-45% and not more than about 65-85% of the total JAK inhibitor is released at 2.5 hours after complete erosion of the delay component or a portion thereof, such as the delay member; and (iii) not less than about 65-85% of the total JAK inhibitor is released at 5 hours after complete erosion of the delay component or a portion thereof, such as the delay member. In some embodiments, the delayed sustained-release oral drug dosage form is configured to release the JAK inhibitor according to the following: (i) not more than about 30% of the total JAK inhibitor is released at 1 hour after complete erosion of the delay component or a portion thereof, such as the delay member; (ii) not less than about 35% and not more than 75% of the total JAK inhibitor is released at 2.5 hours after complete erosion of the delay component or a portion thereof, such as the delay member; and (iii) not less than about 70% of the total JAK inhibitor is released at 5 hours after complete erosion of the delay component or a portion thereof, such as the delay member.

In some embodiments, release of the JAK inhibitor from the delayed sustained-release oral drug dosage form comprises a zero-order release profile, a first-order release profile, a delayed release profile, a pulsed release profile, an iterative pulsed release profile, or a combination thereof.

In some embodiments, the release of the JAK inhibitor from the delayed sustained-release oral drug dosage form is based on an in vivo release rate. In some embodiments, the release of the JAK inhibitor from the delayed sustained-release oral drug dosage form is based on an in vitro release rate. In some embodiments, the release of the JAK inhibitor is based on an in vitro dissolution technique comprising use of a USP rotating paddle apparatus rotated at about 50 RPM and a test medium comprising 900 mL of 0.05 M potassium phosphate buffer at pH 6.8 and 37° C. In some embodiments, the delay member has an in vitro dissolution rate of about 2% per hour to about 40% per hour based on an in vitro dissolution technique comprising use of a USP rotating paddle apparatus rotated at about 50 RPM and a test medium comprising 900 mL of 0.05 M potassium phosphate buffer at pH 6.8 and 37° C.

In some embodiments, the Tₘₐₓ occurs within about 6 hours, such as within about any of 5.5 hours, 5 hours, 4.5 hours, or 4 hours, after complete erosion of the delay component or a portion thereof, such as the delay member.

In some embodiments, when administered to the human individual the ratio of geometric mean plasma Cₘₐₓ, to Cₘᵢₙ is about 10 to about 100, such as any of about 20 to about 40 or about 20 to about 30.

In some embodiments, when the delayed sustained-release oral drug dosage form is administered the human individual, the release of the JAK inhibitor following complete erosion of the delay component or a portion thereof, such as the delay member, is bioequivalent to XELJANZ IR (immediate release) administered to the human individual twice daily. In some embodiments, when the delayed sustained-release oral drug dosage form is administered the human individual, the release of the JAK inhibitor following complete erosion of the delay component or a portion thereof, such as the delay member, is bioequivalent to XELJANZ XR (extended release) administered to the human individual once daily. In some embodiments, the range of values of a pharmacokinetic parameter of the JAK inhibitor of the delayed sustained-release oral drug dosage form described herein is about 60% to about 145%, such as any of about 65% to about 140%, about 70% to about 135%, about 75% to about 130%, about 80% to about 125%, about 85% to about 120%, or about 90% to about 115%, of the pharmacokinetic parameter of a reference PK curve of the JAK inhibitor of a reference oral drug dosage form. In some embodiments, each of the pharmacokinetic parameters of a desired composite PK profile may have the same or a different acceptable threshold. For example, in some embodiments, the desired composite profile comprises more than one pharmacokinetic parameter, wherein one pharmacokinetic parameter has a larger acceptable threshold than another pharmacokinetic parameter.

### D. Exemplary Delayed Sustained-Release Oral Drug Dosage Forms

In some aspects, provided is a delayed sustained-release oral drug dosage form of a Janus kinase (JAK) inhibitor, the delayed sustained-release oral drug dosage form comprising: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, and wherein the delay component layer prevents the release of the JAK inhibitor from the delayed sustained-release oral drug dosage form for about 2 hours to about 6 hours after administration of the delayed sustained-release oral drug dosage form to a human individual. The sustained-release drug component layer, the delay member layer, and the shell are configured such that: the sustained-release drug component layer is embedded in the shell; the delay member layer is embedded in the shell; the bottom surface of the sustained-release drug component layer is in direct contact with the shell; the side surface of the sustained-release drug component layer is in direct contact with the shell; the top surface of the sustained-release drug component layer is in direct contact with the bottom surface of the delay member layer; a portion of the bottom surface of the delay member layer is in direct contact with the shell; and the side surface of the delay member layer is in direct contact with the shell. The portion of the bottom surface of the delay member layer that is in direct contact with the shell forms a perimeter extending beyond the top surface of the sustained-release drug component layer. The shell has an inset for both the sustained-release drug component layer and the delay member layer such that the components are embedded in the shell. The top surface of the delayed sustained-release oral drug dosage form (the surface from which the JAK inhibitor will be released from the oral drug dosage form) is formed from the shell and the delay member layer. The top surface of the delay member layer and the top surface of the sustained-release drug component are in the shape of a capsule.

In some embodiments, provided is an oral drug dosage form representing a portion of a delayed sustained-release oral drug dosage form of a Janus kinase (JAK) inhibitor described herein, wherein the oral drug dosage form comprises: a sustained-release drug component layer comprising a first erodible material admixed with the JAK inhibitor; and a shell, wherein the sustained-release drug component has a length of about 9 mm to about 9.8 mm, such as about 9.4 mm, a width of about 4.8 mm to about 5.6 mm, such as about 5.2 mm, and a height (thickness) of about 0.7 mm to about 1.5 mm, such as about 1.1 mm, and wherein the shell has a length of about 11.4 mm to about 12.2 mm, such as about 11.8 mm, a width of about 7.2 mm to about 8.0 mm, such as about 7.6 mm, and a height (thickness) of about 1.9 mm to about 3.0 mm, such as about 2.3 mm or about 2.6 mm. In some embodiments, the oral drug dosage form representing a portion of a delayed sustained-release oral drug dosage form of a Janus kinase (JAK) inhibitor described herein does not comprise a delay member.

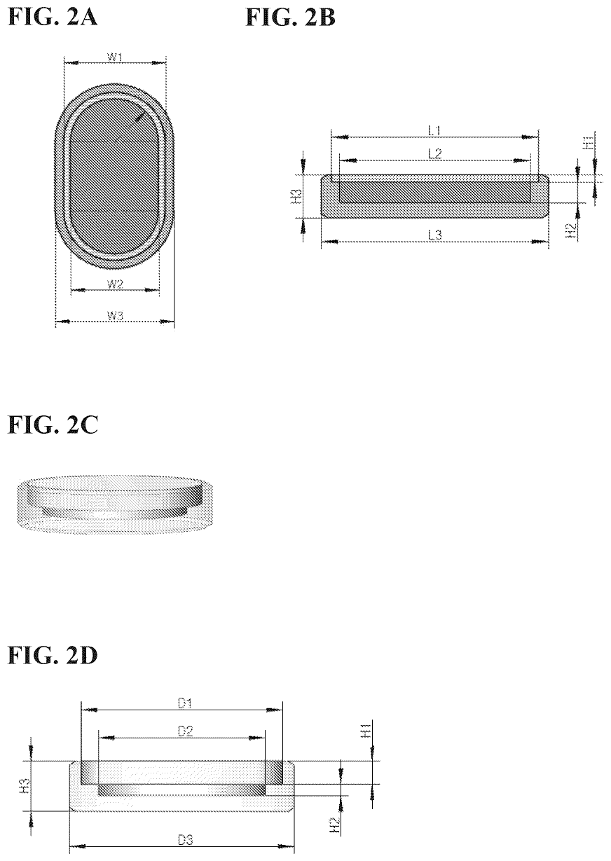

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a length of about 9 mm to about 9.8 mm, such as about 9.4 mm, a width of about 4.8 mm to about 5.6 mm, such as about 5.2 mm, and a height (thickness) of about 0.7 mm to about 1.5 mm, such as about 1.1 mm, wherein the delay member has a length of about 9.8 mm to about 10.6 mm, such as about 10.2 mm, a width of about 5.6 mm to about 6.4 mm, such as about 6.0 mm, and a height (thickness) of about 0.1 mm to about 1.1 mm, such as about 0.4 mm or about 0.7 mm, and wherein the shell has a length of about 11.4 mm to about 12.2 mm, such as about 11.8 mm, a width of about 7.2 mm to about 8.0 mm, such as about 7.6 mm, and a height (thickness) of about 1.9 mm to about 3.0 mm, such as about 2.3 mm or about 2.6 mm. In some embodiments, the delayed sustained-release oral drug dosage form is in the form represented in FIGS. 2A-2B.

In some embodiments, the sustained-release drug component comprises tofacitinib citrate, such as at about 25 wt % to about 45 wt % (e.g., about 35 wt %) relative to the weight of the sustained-release drug component, hydroxypropyl cellulose, such as at about 30 wt % to about 50 wt % (e.g., about 40 wt %) relative to the weight of the sustained-release drug component, vinylpyrrolidone-vinyl acetate copolymer, such as at about 5 wt % to about 15 wt % (e.g., about 10 wt %) relative to the weight of the sustained-release drug component, and glycerin, such as at about 5 wt % to about 25 wt % (e.g., about 15 wt %) relative to the weight of the sustained-release drug component. In some embodiments, the delay member comprises hydroxypropyl cellulose, such as at about 75 wt % to about 95 wt % (e.g., about 85 wt %) relative to the weight of the delay member, triethyl citrate, such as at about 5 wt % to about 25 wt % (e.g., about 15 wt %) relative to the weight of the delay member, and titanium dioxide, such as at about 0.5 wt % to about 0.05 wt % (e.g., about 0.2 wt %) relative to the weight of the delay member.

In some embodiments, the shell comprises ammonio methacrylate copolymer type B, such as at about 55 wt % to about 75 wt % (e.g., about 65 wt %) relative to the shell, ethylcellulose, such as at about 5 wt % to about 25 wt % (e.g., about 15 wt %) relative to the weight of the shell, stearic acid, such as at about 10 wt % to about 30 wt % (e.g., about 20 wt %) relative to the weight of the shell, and titanium dioxide, such as at about 0.5 wt % to about 0.05 wt % (e.g., about 0.2 wt %) relative to the weight of the shell.

In some embodiments, the sustained-release drug component comprises tofacitinib citrate, such as at about 25 wt %/o to about 45 wt % (e.g., about 25 wt %) relative to the weight of the sustained-release drug component, hydroxypropyl cellulose, such as at about 30 wt % to about 50 wt % (e.g., about 40 wt %) relative to the weight of the sustained-release drug component, vinylpyrrolidone-vinyl acetate copolymer, such as at about 5 wt/6 to about 15 wt % (e.g., about 20 wt %) relative to the weight of the sustained-release drug component, and triethyl citrate (TEC), such as at about 5 wt % to about 25 wt % (e.g., about 15 wt %) relative to the weight of the sustained-release drug component.

In some embodiments, the delay member comprises hydroxypropyl cellulose, such as at about 75 wt % to about 95 wt % (e.g., about 90 wt %) relative to the weight of the delay member, triethyl citrate, such as at about 5 wt % to about 25 wt % (e.g., about 9.8 wt %) relative to the weight of the delay member, and titanium dioxide, such as at about 0.5 wt % to about 0.05 wt % (e.g., about 0.2 wt %) relative to the weight of the delay member.

In some embodiments, the shell comprises Ethylcellulose, USP/NF, such as at about 75 wt % to about 85 wt % (e.g., about 80 wt %) relative to the shell, Dibutyl Sebacate(DBS), such as at about 5 wt % to about 25 wt %/o (e.g., about 19.8 wt %) relative to the weight of the shell, and titanium dioxide, such as at about 0.5 wt % to about 0.05 wt % (e.g., about 0.2 wt %) relative to the weight of the shell.

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a diameter of about 7.0 mm to about 10.0 mm, such as about 7.4 mm, and a height (thickness) of about 0.2 mm to about 1.6 mm, such as about 1.4 mm, wherein the delay member has a diameter of about 7.0 mm to about 11.0 mm, such as about 8.2 mm, and a height (thickness) of about 0.2 mm to about 1.2 mm, such as about 0.4 mm, wherein the shell has a diameter of about 9.0 mm to about 11.0 mm, such as about 9.8 mm, and a height (thickness) of about 1.0 mm to about 3.0 mm, such as about 2.2 mm.

In some embodiments, the sustained-release drug component comprises tofacitinib citrate, such as at about 25 wt %/o to about 45 wt % (e.g., about 35 wt %) relative to the weight of the sustained-release drug component, hydroxypropyl cellulose, such as about 30 wt % to about 50 wt % (e.g., about 40 wt %) relative to the weight of the sustained-release drug component, glycerol, such as about 10 wt % to 20 wt % (e.g., about 15 wt %) relative to the weight of the sustained-release drug component, and vinylpyrrolidone-vinyl acetate copolymer, such as at about 5 wt % to about 15 wt % (e.g., about 10 wt %) relative to the weight of the sustained-release drug component.

In some embodiments, the delay member comprises hydroxypropyl cellulose, such as at about 75 wt % to about 95 wt % (e.g., about 85 wt %) relative to the weight of the delay member, and triethyl citrate, such as at about 5 wt % to about 25 wt % (e.g., about 15 wt %) relative to the weight of the delay member.

In some embodiments, the shell comprises ammonium methacrylate copolymer, such as at about 50 wt %/o to about 80 wt % (e.g., about 65 wt %) relative to the shell, ethylcellulose (EC-N10), such as at about 10 wt % to about 20 wt % (e.g., about 15 wt %) relative to the weight of the shell, stearic acid (SA, 95%; SA95), such as at about 10 wt % to about 30 wt % (e.g., about 20 wt %) relative to the weight of the shell, and titanium dioxide, such as at about 0.5 wt % to about 0.05 wt % (e.g., about 0.2 wt %) relative to the weight of the shell

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a length of about 3.0 mm to about 5.0 mm, such as about 4.0 mm, a width of about 4.0 mm to about 5.6 mm, such as about 4.6 mm, and a height (thickness) of about 0.8 mm to about 2.0 mm, such as about 1.35 mm, wherein the delay member has a length of about 3.0 mm to about 5.0 mm, such as about 4.0 mm, a width of about 5.0 mm to about 6.4 mm, such as about 5.4 mm, and a height (thickness) of about 0.2 mm to about 1.2 mm, such as about 0.5 mm, and wherein the shell has a length of about 3.0 mm to about 5.0 mm, such as about 4.0 mm, a width of about 6.4 mm to about 8.0 mm, such as about 7.0 mm, and a height (thickness) of about 1.5 mm to about 3.5 mm, such as about 2.55 mm.

In some embodiments, the sustained-release drug component comprises tofacitinib citrate, such as at about 25 wt % to about 45 wt % (e.g., about 35 wt %) relative to the weight of the sustained-release drug component, hydroxypropyl cellulose, such as about 30 wt % to about 50 wt % (e.g., about 40 wt %) relative to the weight of the sustained-release drug component, glycerol, such as about 10 wt % to 20 wt % (e.g., about 15 wt %) relative to the weight of the sustained-release drug component, and vinylpyrrolidone-vinyl acetate copolymer, such as at about 5 wt % to about 15 wt % (e.g., about 10 wt %) relative to the weight of the sustained-release drug component.

In some embodiments, the delay member comprises hydroxypropyl cellulose, such as at about 75 wt % to about 95 wt % (e.g., about 85 wt %) relative to the weight of the delay member, and triethyl citrate, such as at about 5 wt % to about 25 wt % (e.g., about 15 wt %) relative to the weight of the delay member.

In some embodiments, the shell comprises ammonium methacrylate copolymer, such as at about 50 wt % to about 80 wt % (e.g., about 65 wt %) relative to the shell, ethylcellulose (EC-N10), such as at about 10 wt % to about 20 wt % (e.g., about 15 wt %) relative to the weight of the shell, stearic acid (SA, 95%; SA95), such as at about 10 wt % to about 30 wt % (e.g., about 20 wt %) relative to the weight of the shell, and titanium dioxide, such as at about 0.5 wt % to about 0.05 wt % (e.g., about 0.2 wt %) relative to the weight of the shell.

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a diameter of about 5.0 mm to about 9.0 mm, such as about 8.6 mm, and a height (thickness) of about 0.2 mm to about 1.6 mm, such as about 0.6 mm, wherein the delay member has a diameter of about 7.0 mm to about 11.0 mm, such as about 10.4 mm, and a height (thickness) of about 0.2 mm to about 1.2 mm, such as about 0.8 mm or about 1.2 mm, wherein the shell has a diameter of about 9.0 mm to about 13.0 mm, such as about 11.6 mm, and a height (thickness) of about 1.0 mm to about 3.0 mm, such as about 2.2 mm or about 2.6 mm.

In some embodiments, the sustained-release drug component comprises tofacitinib citrate, such as at about 30 wt % to about 50 wt % (e.g., about 40 wt %) relative to the weight of the sustained-release drug component, hydroxypropyl cellulose, such as about 20 wt % to about 40 wt % (e.g., about 30 wt %) relative to the weight of the sustained-release drug component, PEG400, such as about 10 wt % to 30 wt % (e.g., about 20 wt %) relative to the weight of the sustained-release drug component, and vinylpyrrolidone-vinyl acetate copolymer, such as at about 5 wt % to about 15 wt % (e.g., about 10 wt %) relative to the weight of the sustained-release drug component.

In some embodiments, the delay member comprises hydroxypropyl cellulose, such as at about 75 wt % to about 95 wt % (e.g., about 85 wt %) relative to the weight of the delay member, and PEG400, such as at about 5 wt % to about 25 wt % (e.g., about 15 wt %) relative to the weight of the delay member.

In some embodiments, the shell comprises ammonium methacrylate copolymer, such as at about 50 wt %/o to about 80 wt % (e.g., about 65 wt %) relative to the shell, ethylcellulose (EC-N10), such as at about 10 wt % to about 20 wt % (e.g., about 15 wt %) relative to the weight of the shell, stearic acid (SA, 95%; SA95), such as at about 10 wt % to about 30 wt % (e.g., about 20 wt %) relative to the weight of the shell, and titanium dioxide, such as at about 0.5 wt % to about 0.05 wt % (e.g., about 0.2 wt %) relative to the weight of the shell.

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a diameter of about 5.0 mm to about 9.0 mm, such as about 6.0 mm, about 6.8 mm or about 7.4 mm, and a height (thickness) of about 0.4 mm to about 1.6 mm, such as about 0.8 mm or about 1.4 mm, wherein the delay member has a diameter of about 7.0 mm to about 11.0 mm, such as about 8.4 mm or about 9.4 mm, and a height (thickness) of about 0.2 mm to about 1.2 mm, such as about 0.4 mm or about 1.0 mm.

In some embodiments, the sustained-release drug component comprises tofacitinib citrate, such as at about 25 wt % to about 50 wt % (e.g., about 30 wt % or about 40 wt %) relative to the weight of the sustained-release drug component, hydroxypropyl cellulose, such as about 20 wt % to about 60 wt % (e.g., about 30 wt % or about 55 wt %) relative to the weight of the sustained-release drug component, glycerol, such as about 10 wt % to 25 wt % (e.g., about 15 wt % or about 20 wt %) relative to the weight of the sustained-release drug component, and vinylpyrrolidone-vinyl acetate copolymer, such as at about 0 wt % to about 30 wt % (e.g., about 10 wt % or about 0 wt %) relative to the weight of the sustained-release drug component.

In some embodiments, the delay member comprises hydroxypropyl cellulose, such as at about 75 wt % to about 95 wt % (e.g., about 85 wt %) relative to the weight of the delay member, and triethyl citrate, such as at about 5 wt % to about 25 wt % (e.g., about 15 wt %) relative to the weight of the delay member.

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a length of about 5.0 mm to about 11.0 mm, such as about 5.4 mm, about 8.4 mm or about 11.0 mm, a width of about 4.0 mm to about 5.6 mm, such as about 5.2 mm, and a height (thickness) of about 0.8 mm to about 2.0 mm, such as about 1.2 mm, about 1.35 mm or about 1.8 mm, wherein the delay member has a length of about 5.0 mm to about 11.0 mm, such as about 5.4 mm, about 8.4 mm or about 11.0 mm, a width of about 5.0 mm to about 6.4 mm, such as about 6.0 mm, and a height (thickness) of about 0.2 mm to about 1.2 mm, such as about 0.6 mm or about 0.7 mm, and wherein the shell has a length of about 5.0 mm to about 11.0 mm, such as about 5.4 mm, about 8.4 mm or about 11.0 mm, a width of about 6.4 mm to about 8.0 mm, such as about 7.6 mm, and a height (thickness) of about 2.2 mm to about 3.4 mm, such as about 2.4 mm, about 2.7 mm, or about 2.8 mm.

In some embodiments, the sustained-release drug component comprises tofacitinib citrate, such as at about 15 wt % to about 35 wt % (e.g., about 25 wt %) relative to the weight of the sustained-release drug component, hydroxypropyl cellulose, such as about 30 wt % to about 50 wt % (e.g., about 40 wt %) relative to the weight of the sustained-release drug component, triethyl citrate, such as about 10 wt % to 20 wt % (e.g., about 15 wt %) relative to the weight of the sustained-release drug component, and vinylpyrrolidone-vinyl acetate copolymer, such as at about 10 wt % to about 30 wt % (e.g., about 20 wt %) relative to the weight of the sustained-release drug component.

In some embodiments, the delay member comprises hydroxypropyl cellulose, such as at about 75 wt % to about 95 wt % (e.g., about 90 wt %) relative to the weight of the delay member, triethyl citrate, such as at about 5 wt % to about 25 wt % (e.g., about 10 wt %) relative to the weight of the delay member, and titanium dioxide, such as at about 0.5 wt % to about 0.05 wt % (e.g., about 0.2 wt %) relative to the weight of the delay member.

In some embodiments, the shell comprises ethylcellulose (EC-N10), such as at about 70 wt % to about 90 wt % (e.g., about 80 wt %) relative to the weight of the shell, dibutyl sebacate, such as at about 10 wt % to about 30 wt % (e.g., about 20 wt %) relative to the weight of the shell, and titanium dioxide, such as at about 0.5 wt % to about 0.05 wt % (e.g., about 0.2 wt %) relative to the weight of the shell.

In some embodiments, the delayed sustained-release oral drug dosage form of a Janus kinase (JAK) inhibitor comprises: two sustained-release drug component layers each comprising a first erodible material admixed with the JAK inhibitor; and two delay components each comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a length of about 2.0 mm to about 2.8 mm, such as about 2.4 mm, a width of about 4.0 mm to about 5.6 mm, such as about 4.8 mm, and a height (thickness) of about 0.2 mm to about 1.2 mm, such as about 0.8 mm, wherein the delay member has a length of about 2.0 mm to about 2.8 mm, such as about 2.4 mm, a width of about 5.0 mm to about 6.4 mm, such as about 5.6 mm, and a height (thickness) of about 0.1 mm to about 1.1 mm, such as about 0.4 mm, and wherein the shell has a length of about 2.0 mm to about 2.8 mm, such as about 2.4 mm, a width of about 6.4 mm to about 8.0 mm, such as about 7.2 mm, and a height (thickness) of about 2.2 mm to about 3.4 mm, such as about 2.8 mm.

In some embodiments, the sustained-release drug component comprises tofacitinib citrate, such as at about 15 wt % to about 35 wt % (e.g., about 25 wt %) relative to the weight of the sustained-release drug component, hydroxypropyl cellulose, such as about 30 wt % to about 50 wt % (e.g., about 40 wt %) relative to the weight of the sustained-release drug component, triethyl citrate, such as about 10 wt % to 20 wt % (e.g., about 15 wt %) relative to the weight of the sustained-release drug component, and vinylpyrrolidone-vinyl acetate copolymer, such as at about 10 wt % to about 30 wt % (e.g., about 20 wt %) relative to the weight of the sustained-release drug component.

In some embodiments, the delay member comprises hydroxypropyl cellulose, such as at about 75 wt % to about 95 wt % (e.g., about 90 wt %) relative to the weight of the delay member, and triethyl citrate, such as at about 5 wt % to about 25 wt % (e.g., about 10 wt %) relative to the weight of the delay member.

In some embodiments, the shell comprises ammonium methacrylate copolymer, such as at about 50 wt % to about 80 wt % (e.g., about 65 wt %) relative to the shell, ethylcellulose (EC-N10), such as at about 10 wt % to about 20 wt % (e.g., about 15 wt %) relative to the weight of the shell, stearic acid (SA, 95%; SA95), such as at about 10 wt % to about 30 wt % (e.g., about 20 wt %) relative to the weight of the shell, and titanium dioxide, such as at about 0.5 wt % to about 0.05 wt % (e.g., about 0.2 wt %) relative to the weight of the shell.

In some aspects, provided is a delayed sustained-release oral drug dosage form of a Janus kinase (JAK) inhibitor, the delayed sustained-release oral drug dosage form comprising: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, and wherein the delay component layer prevents the release of the JAK inhibitor from the delayed sustained-release oral drug dosage form for about 2 hours to about 6 hours after administration of the delayed sustained-release oral drug dosage form to a human individual. The sustained-release drug component layer, the delay member layer, and the shell are configured such that: the sustained-release drug component layer is embedded in the shell; the delay member layer is embedded in the shell; the bottom surface of the sustained-release drug component layer is in direct contact with the shell; the side surface of the sustained-release drug component layer is in direct contact with the shell; the top surface of the sustained-release drug component layer is in direct contact with the bottom surface of the delay member layer; a portion of the bottom surface of the delay member layer is in direct contact with the shell; and the side surface of the delay member layer is in direct contact with the shell. The portion of the bottom surface of the delay member layer that is in direct contact with the shell forms a perimeter extending beyond the top surface of the sustained-release drug component layer. The shell has an inset for both the sustained-release drug component layer and the delay member layer such that the components are embedded in the shell. The top surface of the delayed sustained-release oral drug dosage form (the surface from which the JAK inhibitor will be released from the oral drug dosage form) is formed from the shell and the delay member layer. The top surface of the delay member layer and the top surface of the sustained-release drug component are in the shape of a circle. The delayed-sustained release oral drug dosage form in in the shape of a cylinder See, e.g., FIGS. 2C-2D.

In some embodiments, the delayed sustained-release oral drug dosage form of a Janus kinase (JAK) inhibitor comprises: a sustained-release drug component layer comprising a first erodible material admixed with the JAK inhibitor; and a delay component comprising a delay member layer comprising a second erodible material not admixed with the JAK inhibitor; and a shell, wherein the delay component layer surrounds the sustained-release drug component layer, wherein the delay component layer prevents the release of the JAK inhibitor from the delayed sustained-release oral drug dosage form for about 2 hours to about 6 hours after administration of the delayed sustained-release oral drug dosage form to a human individual, wherein the sustained-release drug component has a diameter of about 8.2 mm to about 9.0 mm, such as about 8.6 mm, and a height (thickness) of about 0.2 mm to about 1.0 mm, such as about 0.6 mm, wherein the delay member has a diameter of about 10.0 mm to about 10.8 mm, such as about 10.4 mm, and a height (thickness) of about 0.8 mm to about 1.6 mm, such as about 1.2 mm, and wherein the shell has a diameter of about 11.2 mm to about 12.0 mm, such as about 11.6 mm, and a height (thickness) of about 2.2 mm to about 3.0 mm, such as about 2.6 mm. In some embodiments, the delayed sustained-release oral drug dosage form is in the form represented in FIGS. 2C-2D.

In some embodiments, the sustained-release drug component comprises tofacitinib citrate, such as at about 30 wt % to about 50 wt % (e.g., about 40 wt %) relative to the weight of the sustained-release drug component, hydroxypropyl cellulose, such as at about 20 wt % to about 40 wt % (e.g., about 30 wt %) relative to the weight of the sustained-release drug component, vinylpyrrolidone-vinyl acetate copolymer, such as at about 5 wt % to about 15 wt % (e.g., about 10 wt %) relative to the weight of the sustained-release drug component, and polyethylene glycol 400, such as at about 10 wt % to about 30 wt % (e.g., about 20 wt %) relative to the weight of the sustained-release drug component.

In some embodiments, the delay member comprises hydroxypropyl cellulose, such as at about 75 wt % to about 95 wt % (e.g., about 85 wt %) relative to the weight of the delay member, and polyethylene glycol 400, such as at about 5 wt % to about 25 wt % (e.g., about 15 wt %) relative to the weight of the delay member.

In some embodiments, the shell comprises ammonio methacrylate copolymer type B, such as at about 50 wt % to about 80 wt % (e.g., about 60 wt %) relative to the shell, ethylcellulose, such as at about 10 wt % to about 30 wt % (e.g., about 20 wt %) relative to the weight of the shell, and stearic acid, such as at about 10 wt % to about 30 wt % (e.g., about 20 wt %) relative to the weight of the shell.

In some embodiments, provided herein is a delayed sustained-release oral drug dosage form comprising a delay component housing a sustained-release drug component comprising a plurality of sustained-release beads comprising the JAK inhibitor. In some embodiments, the delay component comprises at least a portion thereof, such as a delay member, configured to release the JAK inhibitor from the delayed sustained-release oral drug dosage form at a desired time following administration. In some embodiments, the sustained-release beads comprise a homogenous mixture comprising tofacitinib, or a pharmaceutical salt thereof and one or more agents to control the release of tofacitinib therefrom.

In some embodiments, provided herein is a delayed sustained-release oral drug dosage form comprising a delay component housing a sustained-release drug component comprising a plurality of sustained-release beads comprising the JAK inhibitor, wherein the sustained-release beads are coated with an agent to control release of the JAK inhibitor thereform. In some embodiments, the delay component comprises at least a portion thereof, such as a delay member, configured to release the JAK inhibitor from the delayed sustained-release oral drug dosage form at a desired time following administration. In some embodiments, the sustained-release beads comprise a homogenous mixture comprising tofacitinib, or a pharmaceutical salt thereof, and one or more agents to control the release of tofacitinib therefrom.

In some embodiments, provided herein is a delayed sustained-release oral drug dosage form comprising a delay component coating a sustained-release drug component comprising the JAK inhibitor. In some embodiments, the delay component is a coating that dissolves after a desired amount of time following administration to the human individual. In some embodiments, the sustained-release drug component is a core comprising tofacitinib, or a pharmaceutical salt thereof, wherein the tofacitinib is homogenously dispersed throughout the core.

In some embodiments, provided herein is a delayed sustained-release oral drug dosage comprising a delay component, a sustained-release drug component, and a gastric retention feature, such as a void space. In some embodiments, the delay component comprises at least a portion thereof, such as a delay member, configured to release the JAK inhibitor from the delayed sustained-release oral drug dosage form at a desired time following administration. In some embodiments, the delay component forms the gastric retention feature and houses the sustained-release drug component. In some embodiments, the portion of the delay component configured to release the JAK inhibitor, such as the delay member, is a plug, such as an erodible plug.

In some embodiments, provided herein is a delayed sustained-release oral drug dosage form comprising a delay component housing a sustained-release drug component comprising the JAK inhibitor. In some embodiments, the delayed sustained-release oral drug dosage form is an osmotic dosage form. In some embodiments, the delay component, or a portion thereof, comprises a material that is permeable to a bodily fluid. In some embodiments, the delay component, or a portion thereof, comprises a material that is selectively permeable. In some embodiments, the sustained-release drug component comprises a plurality of sustained-release beads comprising the JAK inhibitor. In some embodiments, the delay component comprises at least a portion thereof, such as a delay member, configured to release the JAK inhibitor from the delayed sustained-release oral drug dosage form at a desired time following administration. In some embodiments, the sustained-release beads comprise a homogenous mixture comprising tofacitinib, or a pharmaceutical salt thereof, and one or more agents to control the release of tofacitinib therefrom. In some embodiments, the sustained-release beads are coated, such as coated with an agent that controls the release of the JAK inhibitor.

### III. Commercial Batch

In some aspects, provided herein is a commercial batch of at least about 100 delayed sustained-release oral drug dosage forms described herein. In some embodiments, the commercial batch comprises at least about any of 250, 500, 750, 1,000, 2,500, 5,000, 7,500, 10,000, 20,000, 30,000, 40,000, 50,000, 60,000, 70,000, 80,000, 90,000 or 100,000 delayed sustained-release oral drug dosage forms described herein.

In some embodiments, the commercial batch has a standard deviation of about 0.1 or less, such as 0.05 or less, for one or more of the following: an amount of a JAK inhibitor in the delayed sustained-release oral drug dosage form; weight of the delayed sustained-release oral drug dosage form; a largest crossing dimension of the delayed sustained-release oral drug dosage form; and a crossing dimension perpendicular to the largest crossing dimension of the delayed sustained-release oral drug dosage form.

### IV. Methods of Making Delayed Sustained-Release Oral Drug Dosage Forms

In some aspects, provided herein are methods of making a delayed sustained-release oral drug dosage form described herein. In some embodiments, the method of making comprises a three-dimensional (3D) printing technique to form at least one of the components, or a portion thereof, of the delayed sustained-release oral drug dosage forms described herein. In some embodiments, the method of making comprises an injection molding technique to form at least one of the components, or a portion thereof, of the delayed sustained-release oral drug dosage forms described herein.

In some embodiments, provided is a method of three-dimensional (3D) printing of a delayed sustained-release oral drug dosage form described herein, the method comprising dispensing materials according to a layer-by-layer model of the delayed sustained-release oral drug dosage form to print the delayed sustained-release oral drug dosage form, wherein each layer of the layer-by-layer model is printed by dispensing, as necessary, for a layer: (a) a sustained-release drug component comprising a first erodible material admixed with a JAK inhibitor; (b) a delay member comprising a second erodible material not admixed with the JAK inhibitor; and (c) a shell. In some embodiments, the method further comprises generating the layer-by-layer model of the oral drug dosage form. In some embodiments, the dispensing is via melt extrusion deposition (MED). In some embodiments, dispensing of each material is performed by a different printing head.

In some embodiments, provided is a method for preparing a delayed sustained-release tofacitinib oral drug dosage form by three-dimensional (3D) printing, wherein the delayed sustained-release tofacitinib oral drug dosage form comprises a shell containing an insoluble material, a pharmaceutical core containing tofacitinib, and a delay member without tofacitinib, the method comprising dispensing materials according to a layer-by-layer model of the delayed sustained-release oral drug dosage form to print the delayed sustained-release oral drug dosage form, wherein each layer of the layer-by-layer model is printed by dispensing, as necessary, for a layer: (a) a pharmaceutical core containing tofacitinib; (b) the delay member without tofacitinib; and (c) the shell comprising an insoluble material.

As used herein, “printing,” “three-dimensional printing,” “3D printing,” “additive manufacturing,” or equivalents thereof, refers to a process that produces three-dimensional objects, such as delayed sustained-release oral drug dosage forms, layer-by-layer using digital designs. The basic process of three-dimensional printing has been described in U.S. Pat. Nos. 5,204,055; 5,260,009; 5,340,656; 5,387,380; 5,503,785; and 5,633,021. Additional U.S. patents and patent applications that related to three-dimensional printing include: U.S. Pat. Nos. 5,490,962; 5,518,690; 5,869,170; 6,530,958; 6,280,771; 6,514,518; 6,471,992; 8,828,411; U.S. Publication Nos. 2002/0015728; 2002/0106412; 2003/0143268; 2003/0198677; 2004/0005360. The contents of the above U.S. patents and patent applications are hereby incorporated herein by reference in their entirety. In some embodiments, an additive manufacturing technique is used to produce the oral drug dosage forms described herein. In some embodiments, a layer-by-layer technique is used to produce the oral drug dosage forms described herein. Because 3D printing may handle a range of pharmaceutical materials and control both composition and architecture locally, 3D printing is well suited to the fabrication of oral drug dosage forms with complex geometry and compositions in accordance with the present invention.

In some embodiments, layer, when used in reference to, e.g., a sustained-release drug component layer or a delay member layer, refers to the configuration of a component of the oral drug dosage form and may comprise a plurality of printed layers of the same material. In some embodiments, the layer has a pre-determined fill density, such a three-dimensional printed fill density. In some embodiments, the layer, such as the sustained-release drug component layer or the delay member layer, comprises a plurality of printed layers between about 5 printed layers to about 2500 printed layers, such as between any of about 10 printed layers to about 2500 printed layers, about 25 printed layers to about 100 printed layers, about 50 printed layers to about 200 printed layers, about 100 printed layers to about 200 printed layers, about 150 printed layers to about 250 printed layers, about 200 printed layers to about 250 printed layers, about 500 printed layers to about 1000 printed layers, or about 2000 printed layers to about 2400 printed layers. In some embodiments, the thickness of a printed layer is no more than about 5 mm, such as no more than about any of 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm. In some embodiments, the thickness of a printed layer is about any of 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm.

Different 3D printing methods have been developed for manufacturing in terms of raw materials, equipment, and solidification. These 3D printing methods include binder deposition (see Gibson et al., Additive Manufacturing Technologies: 3D Printing, Rapid Prototyping, and Direct Digital Manufacturing., 2 ed. Springer, New York, 2015; Katstra et al., Oral dosage forms fabricated by three dimensional printing, J Control Release, 66, 2000; Katstra et al., Fabrication of complex oral delivery forms by three dimensional printing, Dissertation in Materials Science and Engineering, Massachusetts Institute of Technology, 2001: Lipson et al., Fabricated: The New World of 3D printing, John Wiley & Sons, Inc., 2013; Jonathan, Karim 3D printing in pharmaceutics: a new tool for designing customized drug delivery systems, Int J Pharm, 499, 2016), material jetting (see Jonathan, Karim, 3D printing in pharmaceutics: a new tool for designing customized drug delivery systems, Int J Pharm, 499, 2016), extrusion (see Gibson et al., Additive Manufacturing Technologies: 3D Printing, Rapid Prototyping, and Direct Digital Manufacturing. 2 ed. Springer, New York, 2015), and photopolymerization (see Melchels et al., A review on stereolithography and its application in biomedical engineering. Biomaterials, 31, 2010).

In some embodiments, the oral drug dosage forms described herein are 3D printed using an extrusion method. In some embodiments, the method of 3D printing comprises using a double screw extrusion method. In an extrusion process, material is extruded from robotically-actuated printing heads through printing nozzles. Unlike binder deposition, which requires a powder bed, extrusion methods can print on any substrate. A variety of materials can be extruded for three-dimensional printing, including thermoplastic materials disclosed herein, pastes and colloidal suspensions, silicones, and other semisolids. One extrusion printing method is melt extrusion deposition (MED), which used extruded material from a printing head to print layers of material to form the components of the oral drug dosage form. Another common type of extrusion printing is fused deposition modeling, which uses solid polymeric filaments for printing. In fused deposition modeling, a gear system drives the filament into a heated nozzle assembly for extrusion (see Gibson el al., Additive Manufacturing Technologies: 3D Printing, Rapid Prototyping, and Direct Digital Manufacturing, 2 ed. Springer, New York, 2015).

In some embodiments, the 3D printing is carried out by melt extrusion deposition (MED). In some embodiments, the melt extrusion deposition technique comprises preparing a material to be dispensed, such as preparing a powder in a hot melt extruder, and then feeding the material into a MED printing head. The MED printing head then dispenses the material to form the delayed sustained-release oral drug dosage form in an additive manner (layer-by-layer deposition). In some embodiments, each material of the oral drug dosage form, such as the sustained-release drug component, the delay member, and the shell, is dispensed from a different MED printing head. In some embodiments, the MED printing head dispenses the material according to instructions complied in one or more gcode files. Exemplary MED techniques are disclosed in, e.g., WO2018/210183, WO2019/137333, WO2018137686, and U.S. Pat. No. 10,201,503, each of which is incorporated herein by reference in its entirety.

In some embodiments, the melt extrusion deposition 3D printing technique comprises: (a) preparing each component material by melting and extrusion of the component material, wherein the components comprise a sustained-release drug component, a delay member, and a shell; and (b) printing a delayed sustained-release oral drug dosage form. In some embodiments, the melt extrusion deposition 3D printing technique further comprises preparing a printer head for printing. In some embodiments, preparing the sustained-release drug component comprises melting and extrusion of the component material. In some embodiments, preparing the sustained-release drug component comprises mixing the ingredients of the component material. In some embodiments, preparing the sustained-release drug component comprises weighing each ingredient of the component. In some embodiments, the ingredients of the sustained-release drug component comprise tofacitinib citrate, hydroxypropyl cellulose, vinylpyrrolidone-vinyl acetate copolymer (e.g., copovidone), and glycerin. In some embodiments, preparing the delay member comprises weighing each ingredient of the delay member, mixing the ingredients, and melting and extrusion of the formed delay member material. In some embodiments, the ingredients of the delay member comprise hydroxypropyl cellulose, triethyl citrate, and titanium dioxide. In some embodiments, preparing the shell comprises weighing each ingredient of the shell, mixing the ingredients, and melting and extrusion of the formed shell material. In some embodiments, the ingredients of the shell comprise ammonio methacrylate copolymer type B, ethylcellulose, stearic acid, and titanium dioxide. In some embodiments, preparing the printer head for printing comprises loading the formed component material into the printer head. In some embodiments, preparing the printer head for printing comprises setting the printer head temperature. In some embodiments, preparing the printer head for printing comprises setting and applying the feeding pressure to the printer head. In some embodiments, applying the feeding pressure is completed after the printer head temperature is at a pre-determined level. In some embodiments, printing of the delayed sustained-release oral drug dosage form is performed layer-by-layer (e.g., additive manufacturing). In some embodiments, the method comprises using a separate printer head for each component material (e.g., a first printer head to dispense the sustained-release drug component, a second printer head to dispense the delay member, and a third printer head to dispense the shell).

In some embodiments, the method of making is designed and performed based on a desired total number of delayed-sustained-release oral drug dosage form to be produced in a production run. For example, in some embodiments, a smaller production run (such as less than 1,000 delayed sustained-release oral drug dosage forms for product development or a clinical trial) is desired and the method of making comprises preparing each component material (such as by weighing the ingredients of a component material and forming the component material by mixing the ingredients and hot melt extrusion), and then printing each delayed sustained-release drug dosage form layer-by-layer (e.g., additive manufacturing). In some embodiments, a larger production run (such as a commercial mass production run of more than 1,000 delayed sustained-release oral drug dosage form) is desired and the method of making comprises preparing each component material (such as by weighing the ingredients of a component material and forming the component material by mixing the ingredients and hot melt extrusion, wherein the hot melt extrusion is performed using a twin-screw extruder), and then printing each delayed sustained-release drug dosage form layer-by-layer (e.g., additive manufacturing). In some embodiments, the component material is formed using, at least in part, a twin-screw extruder. In some embodiments, the larger production run comprises distributing the component material from the twin-screw extruder to each printer head via a flow distribution module. In some embodiments, the larger production run is performed via the cooperation of multiple modules. For example, in some embodiments, the method of making comprises use of a system comprising a material supply module for receiving a set of component materials for printing; a flow distribution module comprising a flow distribution plate, wherein the material supply module is configured to transport a single flow corresponding to the set of component materials for printing to the flow distribution plate; wherein the flow distribution plate comprises a plurality of channels for dividing the single flow into a plurality of flows; a plurality of nozzles; and one or more controllers for controlling the plurality of nozzles to dispense the plurality of flows based on a plurality of nozzle-specific parameters. In some embodiments, the system further comprises a printing platform configured to receive the dispensed plurality of flows, wherein the printing platform is configured to move to form a batch of the pharmaceutical product. In some embodiments, the system comprises a plurality of printing platforms.

In some embodiments, the 3D printing is carried out by fused deposition modeling (FDM). In some embodiments, the three-dimensional printing is carried out by melt extrusion deposition or hot melt extrusion coupled with a 3D printing technique, such as FDM. In some embodiments, the 3D printing is carried out by non-filament FDM. In some embodiments, the 3D printing is carried out by inkjet printing. In some embodiments, the 3D printing is carried out by selective laser sintering (SLS). In some embodiments, the 3D printing is carried out by stereolithography (SLA or SL). In some embodiments, the 3D printing is carried out by PolyJet, Multi-Jet Printing System (MJP), Perfactory, Solid Object Ultraviolet-Laser Printer, Bioplotter, 3D Bioprinting, Rapid Freeze Prototyping, Benchtop System, Selective Deposition Lamination (SDL), Laminated Objet Manufacturing (LOM), Ultrasonic Consolidation, ColorJet Printing (CJP), EOSINT Systems, Laser Engineered Net Shaping (LENS) and Aerosol Jet System, Electron Beam Melting (EBM), Laser CUSING®, Selective Laser Melting (SLM), Phenix PXTM Series, Microsintering, Digital Part Materialization (DPM), or VX System.

In some embodiments, the 3D printing methods described herein comprise a continuous feed method. In some embodiments, the 3D printing methods described herein comprise a batch feed method.

In some embodiments, the methods for producing the oral drug dosage forms described herein comprise a 3D printing technique, such as 3D printing in combination with another method, e.g., a combination of injection molding and 3D printing. In some embodiments, the shell is produced using injection molding and one or more modulated-release portions is produced using a 3D printing technique.

The method instructions for 3D printing a drug dosage form disclosed herein may be generated a variety of ways, including direct coding, derivation from a solid CAD model, or other means specific to the 3D printing machine's computer interface and application software. These instructions may include information on the number and spatial placement of droplets, and on general 3D print parameters such as the drop spacing in each linear dimension (X, Y, Z), and volume or mass of fluid per droplet. For a given set of materials, these parameters may be adjusted in order to refine the quality of structure created. The overall resolution of the structure created is a function of the powder particle size, the fluid droplet size, the print parameters, and the material properties.

In some embodiments, one or more components of the delayed sustained-release oral drug dosage form are created separately, such as printed separately, and later assembled to form the oral drug dosage form. In some embodiments, all components of the delayed sustained-release oral drug dosage form are created in a single method, such as printed in a single method, without requiring later assembly.

The delayed sustained-release oral drug dosage forms and components thereof described in the present application can be printed on a commercial scale. For example, in some embodiments, the methods disclosed herein may be used to 3D print 10,000 to 100,000 units of a delayed sustained-release oral drug dosage form per hour. In some embodiments, the methods disclosed herein may be used to 3D print 10,000 to 100,000 oral drug dosage forms per hour. In some embodiments, the methods disclosed herein may be used to 3D print 10,000 to 100,000 units of a dosage unit per hour. In some embodiments, the methods disclosed herein may be used to 3D print 10,000 to 100,000 dosage units per hour.

In some embodiments, the materials used to print the oral drug dosage forms and dosage units, or components thereof, e.g., precursor drug dosage forms, are each dispensed by a different printing head. For example, in some embodiments, the IR material and the ER material, and optionally if present, the intermediate material and the shell material, are each dispensed by a different printing head.

The 3D printing methods described herein encompass printing the materials in any order that will allow for production of the oral drug dosage form and dosage units, or components thereof, e.g., precursor drug dosage forms, disclosed herein.

In some embodiments, the method for 3D printing comprises designing the oral drug dosage form or dosage unit, or component thereof, e.g., a precursor drug dosage form, in whole or in part, on a computer system. In some embodiments, the method comprises inputting parameters of the desired drug release profile and/or the oral drug dosage form and/or the dosage unit and/or a precursor drug dosage form into the computer system. In some embodiments, the method comprises providing one or more parameters to be printed, e.g., layer surface area, thickness, drug mass fraction, erosion rate. In some embodiments, the method comprises providing the desired drug release profile. In some embodiments, the methods comprise creating a virtual image of the item to be printed. In some embodiments, the method comprises creating a computer model that contains the pre-determined parameters. In some embodiments, the method comprises feeding the pre-determined parameters to a 3D printer and printing the item according to such pre-determined parameters. In some embodiments, the method comprises creating a 3D drawing of the item to be printed based on the pre-determined parameters, wherein the 3D drawing is created on a computer system. In some embodiments, the method comprises converting, such as slicing, a 3D drawing into 3D printing code, e.g., G code. In some embodiments, the method comprises using the computer system to execute 3D printing code, thereby printing according to the methods described herein.

In some embodiments, provided herein is a method of three-dimensional (3D) printing of a delayed sustained-release oral drug dosage form described herein, the method comprising: (a) dispensing the delay component or a portion thereof; and (b) dispensing the sustained-release drug component comprising the first erodible material admixed with the JAK inhibitor. In some embodiments, the delay component, or a portion thereof, such as the delay member or the shell, is dispensed prior to the dispensing of the sustained-release drug component. In some embodiments, the delay component, or a portion thereof, such as the delay member or the shell, is dispensed after the dispensing of the sustained-release drug component. In some embodiments, dispensing the delay component comprises: (i) dispensing the shell; and (ii) dispensing the delay member comprising the second erodible material not admixed with the JAK inhibitor. In some embodiments, dispensing is via melt extrusion deposition (MED). In some embodiments, dispensing of the delay component, such as dispensing of the shell and dispensing of the delay member are performed by a different printing head.

In some embodiments, provided herein is a method for preparing a delayed sustained-release tofacitinib oral drug dosage form by three-dimensional (3D) printing, wherein the delayed sustained-release tofacitinib oral drug dosage form comprises a shell, a sustained-release dug component comprising tofacitinib, and a delay member without tofacitinib, the method comprising: (a) dispensing the shell; (b) dispensing the sustained-release component comprising tofacitinib; and (c) dispensing the delay member without tofacitinib. Using the method, the components of the delayed sustained-release oral drug dosage forms, or portions thereof, may be dispensed in any order. In some embodiments, each component is dispensed, to completion, in sequence in a specified order. In some embodiments, layers of the delayed sustained-release oral drug dosage form is formed in layers, wherein each layer comprises one or more components of the oral drug dosage form. In some embodiments, the method comprises dispensing, in the following order, the shell, the sustained-release component, and the delay member. In some embodiments, the method comprises dispensing, in the following order, the delay member, the sustained-release component, and the shell. In some embodiments, the dispensing is via melt extrusion deposition (MED). In some embodiments, dispensing of each material is performed by a different printing head.

In some embodiments, provided herein is a method of forming a delayed sustained-release oral drug dosage form via injection molding. In some embodiments, provided is a method of injection molding an oral drug dosage form of any one of claims 1-57, the method comprising: (a) injecting a hot melt of the shell material into a mold cavity to form the shell; (b) injecting a hot melt of the first erodible material admixed with a JAK inhibitor into the shell to form the sustained-release drug component; and (c) injecting a hot melt of the second erodible material not admixed with the JAK inhibitor into the shell to form the delay member.

In some embodiments, provided herein is a method of injection molding a delayed sustained-release oral drug dosage form described herein, the method comprising: (a) hot melting a shell material, a first erodible material admixed with a JAK inhibitor, and a second erodible material not admixed with the JAK inhibitor; (b) delivering each material to the respective injection unit; (c) injecting a hot melt of the shell material into a mold cavity to form the shell; (d) allowing the shell to cool and opening the mold to release the shell; (e) transferring the shell to a male mold to inject the first erodible material admixed with the JAK inhibitor to form the sustained-release drug component; (f) injection a hot melt of the first erodible material admixed with the JAK inhibitor to form the sustained-release drug component: (g) allowing the sustained-released drug component to cool and opening the mold to release the shell and the sustained-release drug component; (h) transferring the shell and the sustained-release drug component to a male mold to inject the second erodible material not admixed with the JAK inhibitor to form the delay member; (i) injection a holt melt of the second erodible material not admixed with the JAK inhibitor; and j) ejecting the delayed sustained-release oral drug dosage form. In some embodiments, the injection unit is selected from the group consisting of a single screw injection unit, a plunger injection unit, and a gear pump injection unit. In some embodiments, step (c) to step j) are performed in series. In some embodiments, step (c), step (f), and step (i) are performed at the same time. In some embodiments, step (e), step (h), and step j) are performed at the same time.

In some aspects, the methods provided comprise preparing the material of the dosage form (e.g., the material for each of the sustained-release drug component, the delay member, and the shell), making the dosage form (such as via printing, e.g., 3D printing), and one or more packaging steps. In some embodiments, preparing the material of the dosage form comprises weighing each of the ingredients for the material. In some embodiments, the packaging step comprises packaging each individual dosage form into a discrete container, such as laminated film and pouches for pharmaceutical packaging. In some embodiments, the packaging step comprises packaging a number of packaged dosage forms into a carton. In some embodiments, the method further comprises one or more in-process quality control steps. For example, in some embodiments, after making the dosage form, the in-process quality control step comprises one or more of assessing a dosage form for appearance or a feature thereof, assessing the dosage form for weight, and assessing the dosage form for dimensions. In some embodiments, to pass the in-process quality control step, the evaluated characteristic must be within a pre-determined threshold. In some embodiments, after each dosage for is packaged, the in-process quality control step comprises assess the seal of each dosage form, e.g., for tightness, and/or filing quantity. In some embodiments, after packaging in the carton, the in-process quality control step comprises confirming the filing quantity of the carton.

### V. Methods of Treating and/or Preventing

In some aspects, provided herein are methods of treating and/or preventing conditions comprising administering a delayed sustained-release oral drug dosage forms described herein. In some embodiments, the methods comprise a once daily administration of a delayed sustained-release oral drug dosage form described herein.

In some embodiments, provided is a method for preventing morning stiffness, the method comprising administering to a human individual a delayed sustained-release oral drug dosage form described herein, wherein the delayed sustained-release oral drug dosage form is administered the evening prior to the morning for which prevention of morning stiffness is desired. In some embodiments, the delayed sustained-release oral drug dosage form is administered at least about 6 hours, such as at least about any of 7 hours, 8 hours, 9 hours, or 10 hours, prior to the morning for which benefit is desired. In some embodiments, the delayed sustained-release oral drug dosage form is administered within about 4 hours, such as with about any of 3 hours, 2 hours, 1 hour, or 30 minutes, of going to bed to sleep for the evening. In some embodiments, the method comprises a once daily administration of a delayed sustained-release oral drug dosage form described herein.

In some embodiments, provided is a method for preventing morning stiffness caused by rheumatoid arthritis, the method comprising administering to a human individual a delayed sustained-release oral drug dosage form described herein, wherein the delayed sustained-release oral drug dosage form is administered the evening prior to the morning for which prevention of morning stiffness is desired. In some embodiments, the delayed sustained-release oral drug dosage form is administered at least about 6 hours, such as at least about any of 7 hours, 8 hours, 9 hours, or 10 hours, prior to the morning for which benefit is desired. In some embodiments, the delayed sustained-release oral drug dosage form is administered within about 4 hours, such as with about any of 3 hours, 2 hours, 1 hour, or 30 minutes, of going to bed to sleep for the evening. In some embodiments, the method comprises a once daily administration of a delayed sustained-release oral drug dosage form described herein.

In some embodiments, provided is a method for preventing morning stiffness caused by psoriatic arthritis, the method comprising administering to a human individual a delayed sustained-release oral drug dosage form described herein, wherein the delayed sustained-release oral drug dosage form is administered the evening prior to the morning for which prevention of morning stiffness is desired. In some embodiments, the delayed sustained-release oral drug dosage form is administered at least about 6 hours, such as at least about any of 7 hours, 8 hours, 9 hours, or 10 hours, prior to the morning for which benefit is desired. In some embodiments, the delayed sustained-release oral drug dosage form is administered within about 4 hours, such as with about any of 3 hours, 2 hours, 1 hour, or 30 minutes, of going to bed to sleep for the evening. In some embodiments, the method comprises a once daily administration of a delayed sustained-release oral drug dosage form described herein.

In some embodiments, provided is a method for treating ulcerative colitis, the method comprising administering to a human individual a delayed sustained-release oral drug dosage form described herein. In some embodiments, provided is a method for preventing and/or reducing symptoms associated with ulcerative colitis, such as symptoms that occur in the morning, the method comprising administering to a human individual a delayed sustained-release oral drug dosage form described herein. In some embodiments, the delayed sustained-release oral drug dosage form is administered the evening prior to the morning for which the benefit is desired. In some embodiments, the delayed sustained-release oral drug dosage form is administered at least about 6 hours, such as at least about any of 7 hours, 8 hours, 9 hours, or 10 hours, prior to the morning for which benefit is desired. In some embodiments, the delayed sustained-release oral drug dosage form is administered within about 4 hours, such as with about any of 3 hours, 2 hours, 1 hour, or 30 minutes, of going to bed to sleep for the evening. In some embodiments, the method comprises a once daily administration of a delayed sustained-release oral drug dosage form described herein.

In some embodiments, the dosage form provided herein is administered when the subject is in the fed state. In some embodiments, the dosage form provided herein is administered when the subject is in the fasted state. In some embodiments, the dosage form provided herein show no significant difference in drug dissolution and/or absorption in the fed state compared to the fasted state.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

### Example 1

This example demonstrates the design and testing of two delayed sustained-release oral drug dosage forms comprising a fixed amount of a JAK inhibitor, namely, tofacitinib, having a desired drug release profile.

The two oral drug dosage forms were produced and denoted as caplets A and B in the following. The 3D structures of the caplet oral drug dosage forms were designed using Solidworks 2014. The schematic drawing of the caplets A and B are shown in FIGS. 2A and 2B. The dimensions and component compositions of the prepared dosage forms was as described in the Specification. Caplets A and B were 3D printed using a MED 3D printing system. Both caplet A and B contained 17.77 mg of tofacitinib citrate (equivalent to 11 mg tofacitinib). The delay member was composed of an erodible polymer matrix. The shell was composed of a water-insoluble polymer.

The in vitro dissolution rates of caplets A and B were tested and compared to a marketed tofacitinib containing drug, XELJANZ XR (Pfizer). XELJANZ XR also contains 17.77 mg of tofacitinib citrate (equivalent to 11 mg tofacitinib). Dissolution rates were measured using the same method as that of tofacitinib citrate ER tablet in the current FDA Dissolution Methods Database. Briefly, caplets A and B were dissolved in 900 mL of phosphate buffer at pH 6.8 in drug dissolution apparatus USP II (paddle) at 50 rpm, with the delay member facing downwards in the phosphate buffer. Accumulative percentage of dissolution was measured at one-hour intervals until dissolution of the erodible materials was complete. The dissolution profile of XELJANZ XR was obtained using the same method. The dissolution experiment on each dosage form was repeated six times.

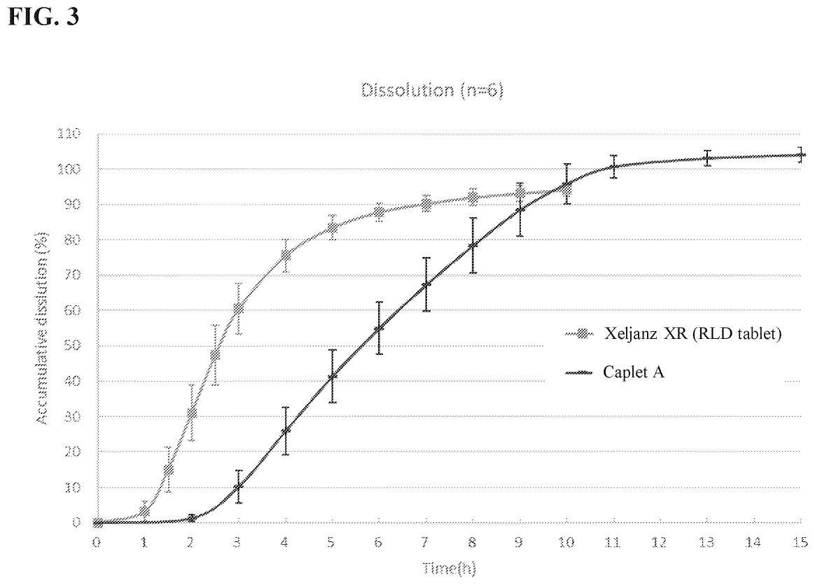

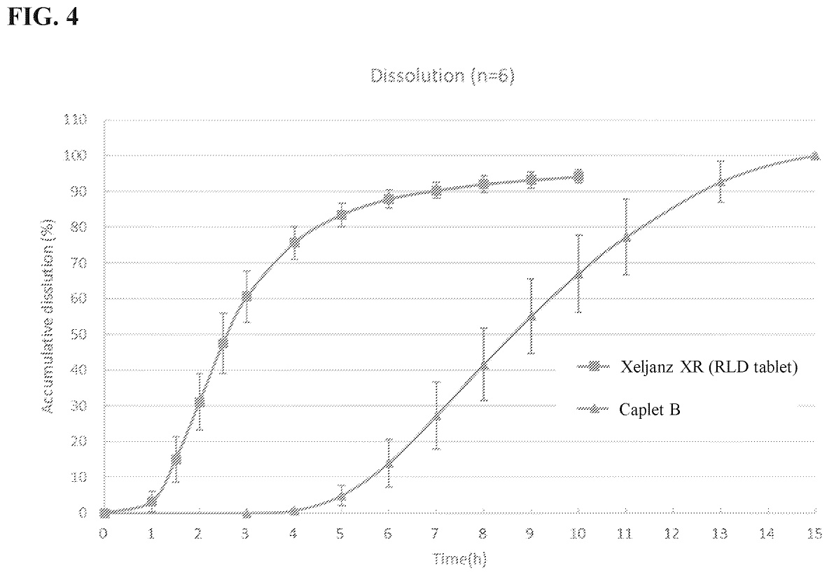

The dissolution profiles of caplets A and B in comparison with XELJANZ XR are shown in FIGS. 3 and 4, respectively. Due to the delayed release feature, both caplets A and B had prolonged dissolution profiles compared to that of XELJANZ XR. XELJANZ XR was 50% dissolved in 2-3 hours, and reached 90% dissolution in 7 hours. Caplet A reached 50% dissolution between 6-7 hours, and 90% between 9-10 hours. Caplet B showed longer delay in dissolution, with 50% dissolution in 8-9 hours and 90% dissolution in 12-13 hours. The results are consistent with the thicker delay member on caplet B (0.7 mm) as compared to the thickness of the delay member on caplet A (0.4 mm).

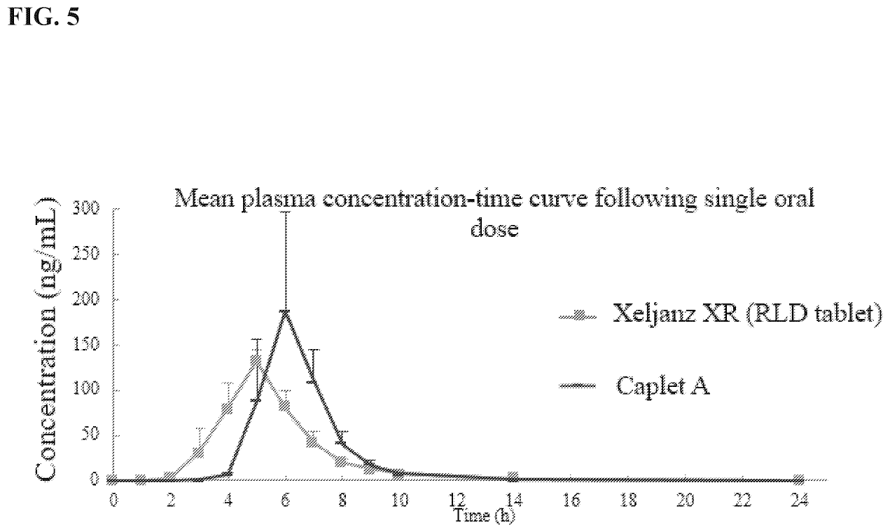

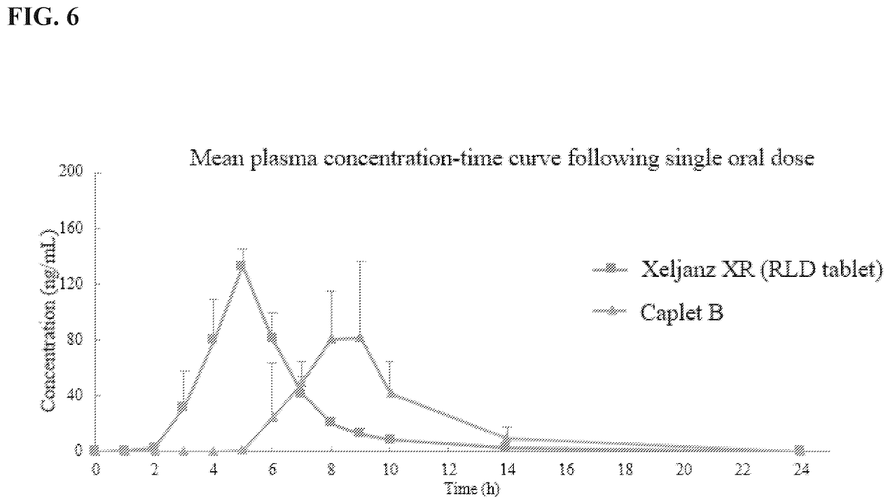

Caplet A, caplet B, and XELJANZ XR (RLD tablet) were subjected to in vivo pharmacokinetic studies in non-GLP fed male beagle dogs. After a single oral administration of the respective drug dosage forms, blood samples were collected from the jugular vein at predetermined times (one-hour intervals up to 24 hours after administration). The plasma concentrations of the drug were determined by LC-MS/MS analysis (n=5 male beagle dogs). The curves of mean plasma concentration for the tested formulations are shown in FIGS. 5 and 6.

Both caplet A and caplet B showed effective delay in plasma Cₘₐₓ in vivo. As shown in FIG. 5, caplet A achieved a desirable, rapid initial increase in plasma drug concentration after a delay. The Tₘₐₓ of caplet A is delayed by approximately 2 hours compared to XELJANZ XR, with minimal change of AUC between the two dosage forms. As shown in FIG. 6, caplet B had a delayed release of tofacitinib of about 3 hours, followed by a prolonged phase of drug delivery. Caplet B reached Cₘₐₓ between 8-9 hours after administration, over 3 hours later than XELJANZ XR.

### Example 2

This example demonstrates the design and testing of a portion of a delayed sustained-release oral drug dosage form comprising a fixed amount of a JAK inhibitor, namely, tofacitinib. The portion of the oral drug dosage form contains a shell and the sustained-release drug component comprising the first erodible material admixed with the JAK inhibitor as used in caplets A and B in Example 1, and was designed without the delay member comprising a second erodible material not admixed with the JAK inhibitor. Such a portion of an oral drug dosage form is useful for studying the release of the JAK inhibitor from the oral drug dosage form without the delay provided by the delay member.

The oral drug dosage form was produced and denoted as caplet C in the following. The 3D structures of the caplet oral drug dosage form was designed using Solidworks 2014. The dimensions and component compositions of the prepared dosage form were as described in the Specification. Caplet C was 3D printed using a MED 3D printing system. Dosage form C contained 17.77 mg of tofacitinib citrate (equivalent to 11 mg tofacitinib). The shell was composed of a water-insoluble polymer.

The in vitro dissolution rates of caplet C were tested and compared to a marketed tofacitinib containing drug, XELJANZ XR (Pfizer), as well as caplets A and B from Example 1. XELJANZ XR and caplets A and B all contained 17.77 mg of tofacitinib citrate (equivalent to 11 mg tofacitinib). Dissolution rates were measured using the same method as that of tofacitinib citrate ER tablet in the current FDA Dissolution Methods Database. Briefly, caplets A, B, and C were dissolved in 900 mL of phosphate buffer at pH 6.8 in drug dissolution apparatus USP II (paddle) at 50 rpm, with the delay member side facing downwards in the phosphate buffer. Accumulative percentage of dissolution was measured at one-hour intervals until dissolution of the erodible materials was complete. The dissolution profile of XELJANZ XR was obtained using the same method. The dissolution experiment on each dosage form was repeated six times.

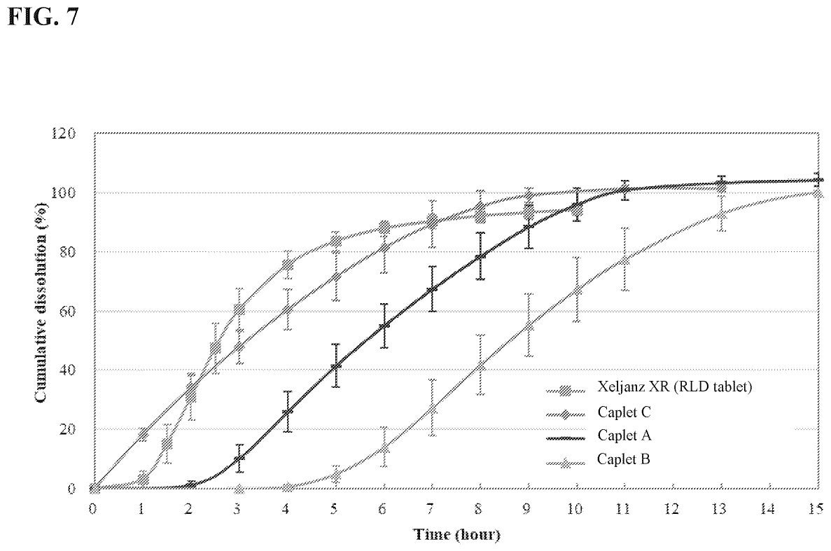

The dissolution profiles of caplets A, B, and C, in comparison with XELJANZ XR, are shown in FIG. 7. Caplet C reached 50% dissolution in 3-4 hours, and was 90% dissolved in 7 hours, similar to XELJANZ XR, which was 50% dissolved in 2-3 hours, and reached 90% dissolution in 7 hours. Adjustments to the sustained-release drug component comprising the first erodible material comprising the JAK inhibitor can be made such that the dissolution profile of caplet C becomes more similar to the dissolution profile of Xeljanz XR Due to the presence of a delay member comprising a second erodible material not admixed with the JAK inhibitor, caplets A and B have prolonged dissolution profiles compared to that of XELJANZ XR and caplet C.

### Example 3

This example demonstrates studies the pharmacokinetics (PK) profiles of caplets A and B (from Example 1) as compared to Xeljanz XR in human subjects.

16 healthy subjects participated in the PK studies. The subjects were divided into 4 groups of 4 to partake in an open, single-dose crossover experiment (4 cycles for each group). The experimental design is shown in Table 1. X represented a version of a simulated “real world” situation wherein the subjects had dinner at 18:00 and a simple meal at 21:30 prior to administration of caplet A. Caplet A was then administered immediately before bed (22:00). Y represented a version of a simulated “real world” situation wherein the subjects had dinner at 18:00 and a simple meal at 21:30 prior to administration of caplet B. Caplet B was then administered immediately before bed (22:00). Z represented a version of a simulated “real world” situation wherein the subjects had dinner at 18:00, and then caplet B was administered immediately before bed (22:00). W represented the control group wherein subjects took XELJANZ XR at 8:00 in the morning after fasting for at least 10 hours. Each group was subjected to a different experimental condition (X, Y, Z, or W) during each cycle of the experiment.

TABLE 1
Experimental design of the PK study using caplets A and B.

| Group | | | | |
|---|---|---|---|---|
| (Total number of | | | | |
| subjects = 16) | Cycle I | Cycle II | Cycle III | Cycle IV |
| 1 (4 subjects) | X | Y | Z | W |
| 2 (4 subjects) | Y | Z | W | X |
| 3 (4 subjects) | Z | W | X | Y |
| 4 (4 subjects) | W | X | Y | Z |

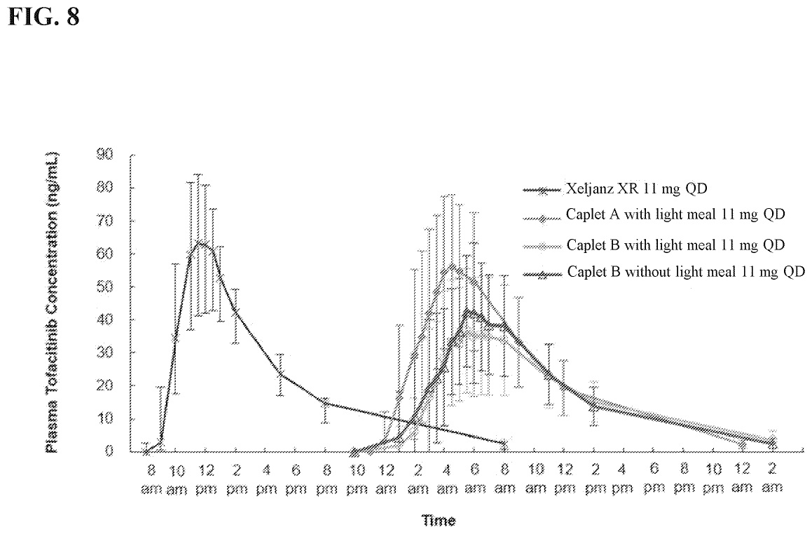

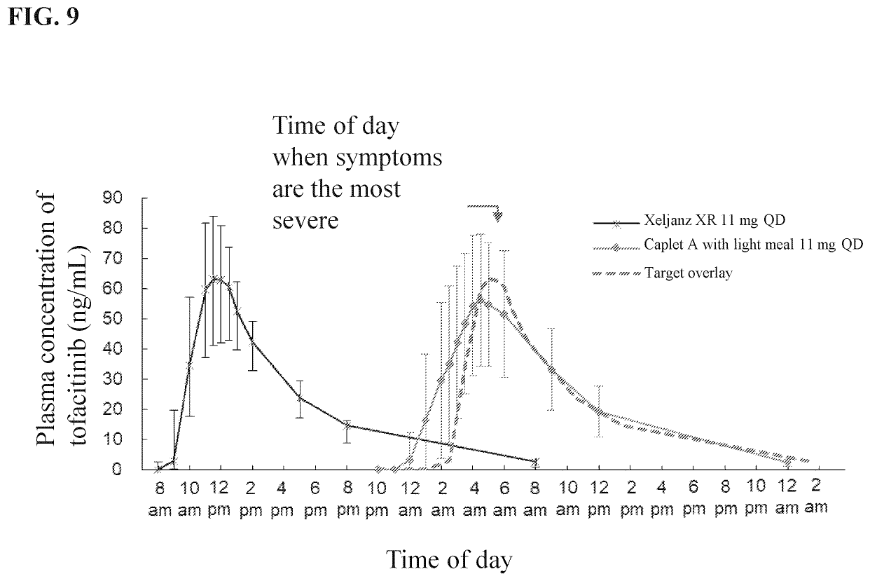

The PK parameters are shown in Table 2 and the resulting PK curves are shown in FIG. 8. FIG. 9 shows the PK curves of Xeljanz XR, the dashed line overlay of the target PK curve (that of Xeljanz XR delayed by 14 hours), and caplet A with light meal at 21:30.

TABLE 2
PK parameters obtained from human subjects.

| PK parameters | | X | Y | Z | W |
|---|---|---|---|---|---|
| (n = 16, | | Caplet A with | Caplet B with | Caplet B without | Xeljanz XR |
| mean ± SD) | Unit | light meal 21:30 | light meal 21:30 | light meal 21:30 | fasting |
| Cₘₐₓ | ng/mL | 68.9 ± 19.8 | 43.5 ± 18.0 | 51.0 ± 20.0 | 68.5 ± 23.2 |
| Tₘₐₓ | h | 5.81 ± 1.39 | 8.16 ± 2.44 | 7.50 ± 1.65 | 3.92 ± 0.80 |
| AUC₀₋ₜ | ng*h/mL | 561 ± 164 | 430 ± 155 | 449 ± 147 | 495 ± 106 |
| AUC_{0-inf} | ng*h/mL | 574 ± 170 | 483 ± 161 | 469 ± 147 | 514 ± 111 |
| T_{1/2} | h | 3.84 ± 0.462 | 5.43 ± 2.09 | 4.74 ± 1.44 | 4.54 ± 1.11 |
| T_{lag} | h | 2.81 ± 0.403 | 4.22 ± 0.875 | 3.88 ± 0.806 | 1.00 ± 0.000 |
| Fᵣₑₗ | % | 110 ± 21.3 | 91.7 ± 29.4 | 92.9 ± 28.3 | — |

The average Cₘₐₓ and Tₘₐₓ in Table 2 were calculated from the Cₘₐₓ and Tₘₐₓ of each of the 16 subjects, while the plasma concentration-time curves showed the average at each time point, therefore the fitted Cₘₐₓ and Tₘₐₓ values from the curve might vary from the average Cₘₐₓ and Tₘₐₓ. From the results above, the delay member comprising an erodible material not admixed with the JAK inhibitor in caplets A and B effectively delayed T_{lag} and Tₘₐₓ. As the thickness of the delay member increased (comparison of caplet A and caplet B), the T_{lag} and Tₘₐₓ increased accordingly. The AUC_{0-inf} and Cₘₐₓ of caplet A were 86.1% (90% confidence interval: 74.9%-98.9%) and 60.7% (90% confidence interval: 49.5%-74.4%) of Xeljanz XR, respectively. The AUC of dosage forms A and B was similar to that of Xeljanz XR. The light meal at 21:30 had no significant impact on absorbance of tofacitinib.

### Example 4

This example demonstrates the design and testing of a delayed sustained-release oral drug dosage forms comprising a fixed amount of a JAK inhibitor, namely, tofacitinib, having a desired drug release and PK profile.

The oral drug dosage form was produced and denoted as caplet 2A in the following. The 3D structures of the caplet oral drug dosage form was designed using Solidworks 2014. The schematic drawing of the caplet 2A is shown in FIGS. 2A and 2B. The dimensions and component compositions of the prepared dosage forms was as described in the Specification. Caplet 2A was 3D printed using a MED 3D printing system, containing 17.77 mg of tofacitinib citrate (equivalent to 11 mg tofacitinib). The delay member was composed of an erodible polymer matrix not comprising the JAK inhibitor. The shell was composed of a water-insoluble polymer.

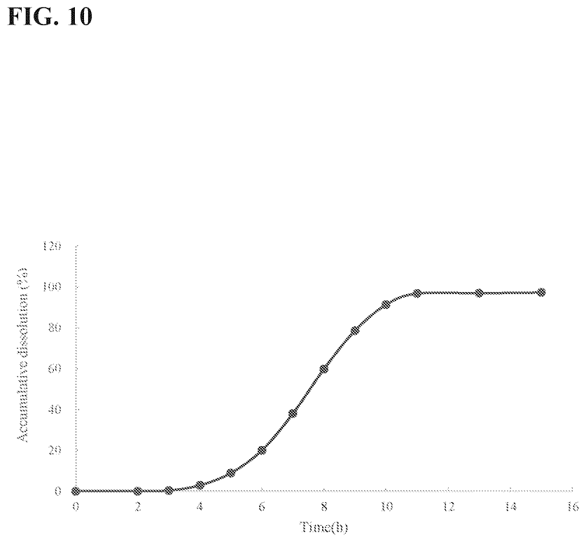

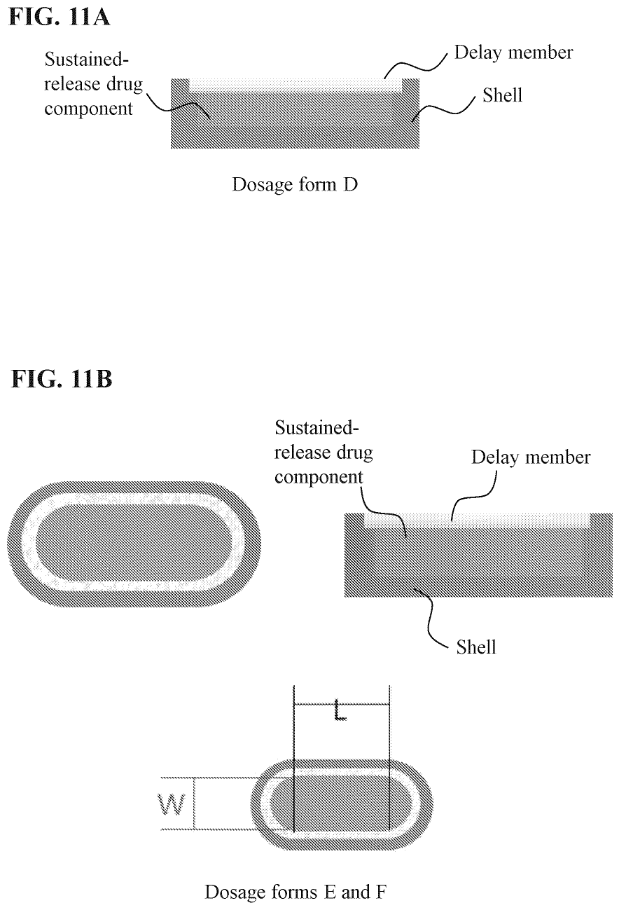

The in vitro dissolution profile of caplet 2A was measured as described in Example 1. The dissolution profile of caplet 2A is shown in FIG. 10. Caplet 2A reached 50% dissolution between 7-8 hours, which was longer than the time to reach 50% dissolution for caplet A (6-7 hours). Caplet 2A reached 90% dissolution between 9-10 hours.

The PK profile of caplet 2A will be obtained on human subjects according to the experimental design as follows. A single-center, randomized, open, single-dose, 2-cycle crossover early pharmacokinetic (PK) clinical trial will be conducted with the experimental design shown in Table 3 below. The subjects will be given dinner at 18:00 followed by administration of a single dose of caplet 2A orally at 22:00, or a single dose of the Xeljanz XR orally in the morning after overnight fasting of at least 10 hours.

TABLE 3
Experimental design of the PK study using dosage form 2A

| Group(n = 12) | Cycle 1 | Cycle 2 |
|---|---|---|
| 1 (n = 6) | 2A | Xeljanz XR |
| 2 (n = 6) | Xeljanz XR | 2A |

### Example 5

This example demonstrates the design and testing of a delayed sustained-release oral drug dosage forms comprising a fixed amount of a JAK inhibitor, namely, tofacitinib, having a desired drug release profile.

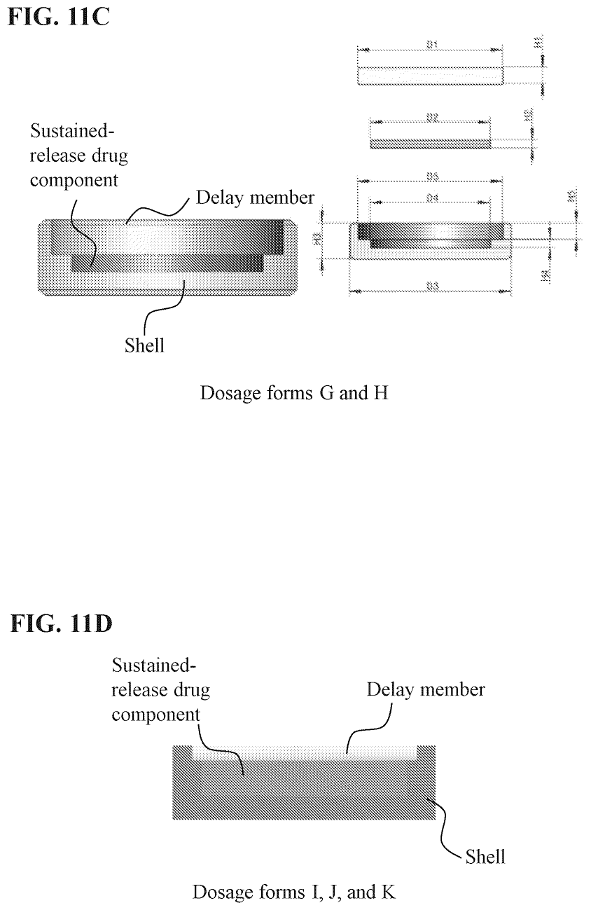

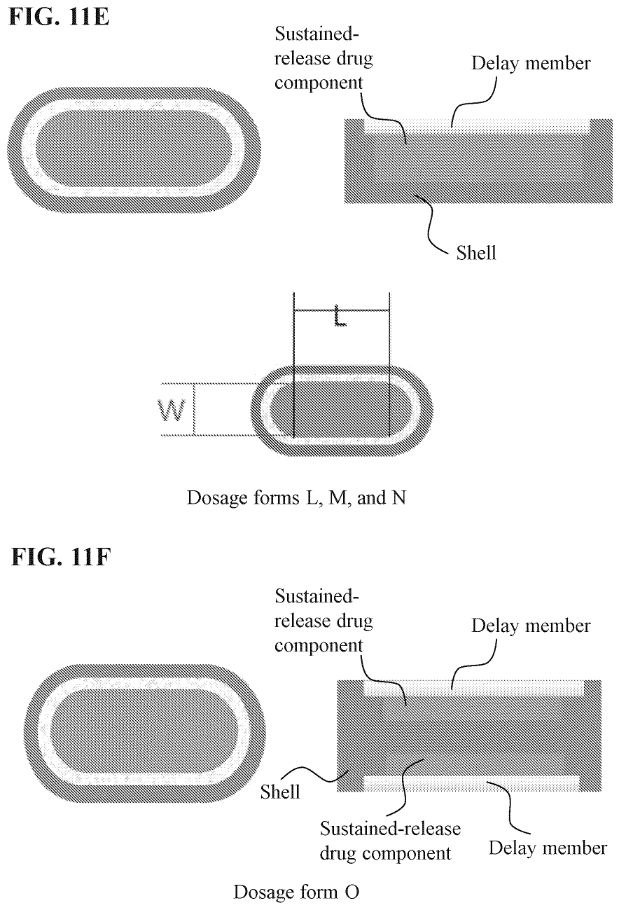

The delayed sustained-release oral drug dosage forms D-O as shown in FIGS. 11A-11F were produced. The dimensions and component compositions of the prepared dosage forms was as described in the Specification. The 3D structures of the oral drug dosage forms was designed using Solidworks 2014. The oral drug dosage forms were 3D printed using a MED 3D printing system. Each oral drug dosage form contained about 17.77 mg of tofacitinib citrate (equivalent to 11 mg tofacitinib). The delay member was composed of an erodible polymer matrix. The shell was composed of a water-insoluble polymer.

The in vitro dissolution rate of dosage form D was tested and compared to a marketed tofacitinib containing drug, XELJANZ XR (Pfizer). XELJANZ XR contains 17.77 mg of tofacitinib citrate (also equivalent to 11 mg tofacitinib). Dissolution rates were measured using the same method as described in Example 1.

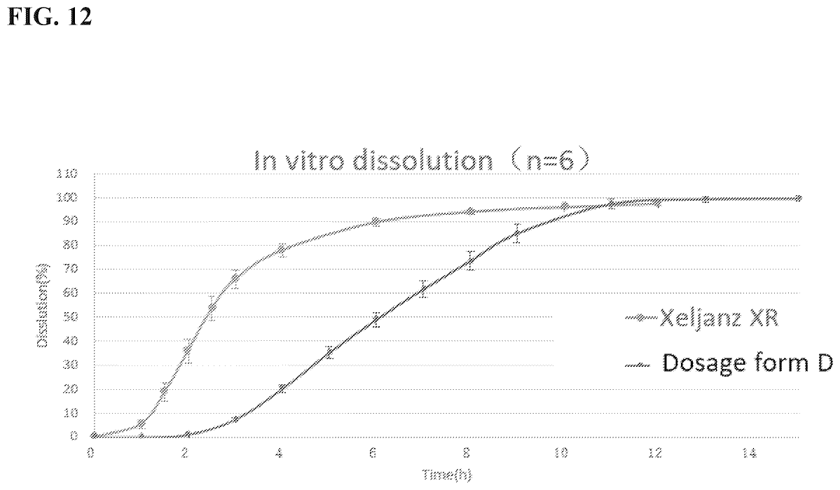

The dissolution profile of dosage form D in comparison with XELJANZ XR is shown in FIG. 12. Due to the delayed release feature, dosage form D had a prolonged dissolution profile compared to that of XELJANZ XR. XELJANZ XR was 50% dissolved in 2-3 hours, and reached 90% dissolution in 7 hours. Dosage form D reached 50% dissolution between 6-7 hours, and 90% around 10 hours.

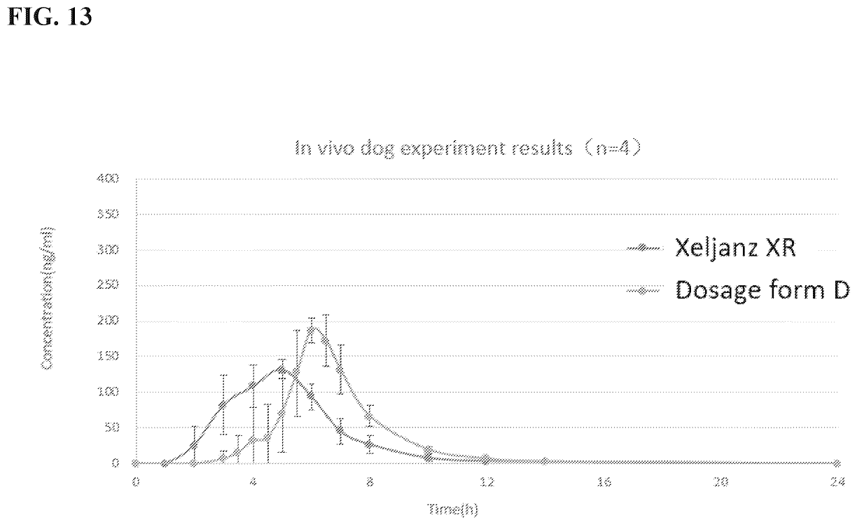

Dosage form D and XELJANZ XR (RLD tablet) were subjected to in vivo pharmacokinetic studies in non-GLP fed male beagle dogs as described in Example 1. The curves of mean plasma concentration for the tested formulations are shown in FIG. 13.

Dosage form D showed effective delay in plasma Cₘₐₓ in vivo. As shown in FIG. 13, caplet E achieved a desirable, rapid initial increase in plasma drug concentration after a delay. The Tₘₐₓ of caplet E was delayed by approximately 2 hours compared to XELJANZ XR, with minimal change of AUC between the two dosage forms.

The in vitro dissolution rates of dosage forms E and F were tested. Dissolution rates were measured using the same method as described in Example 1.

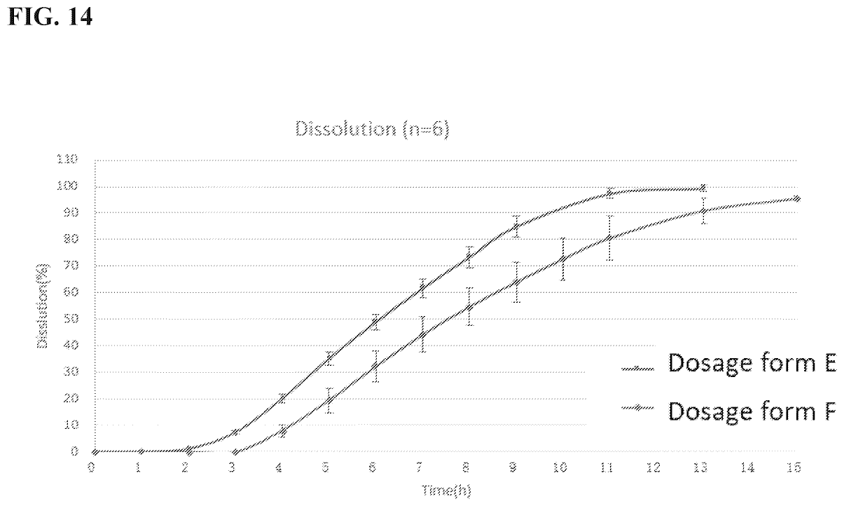

The dissolution profiles of dosage forms E and F are shown in FIG. 14. Dosage form E reached 50% dissolution in around 6 hours, and 90% between 9-10 hours. Dosage form F reached 50% dissolution between 7-8 hours, and 90% around 13 hours.

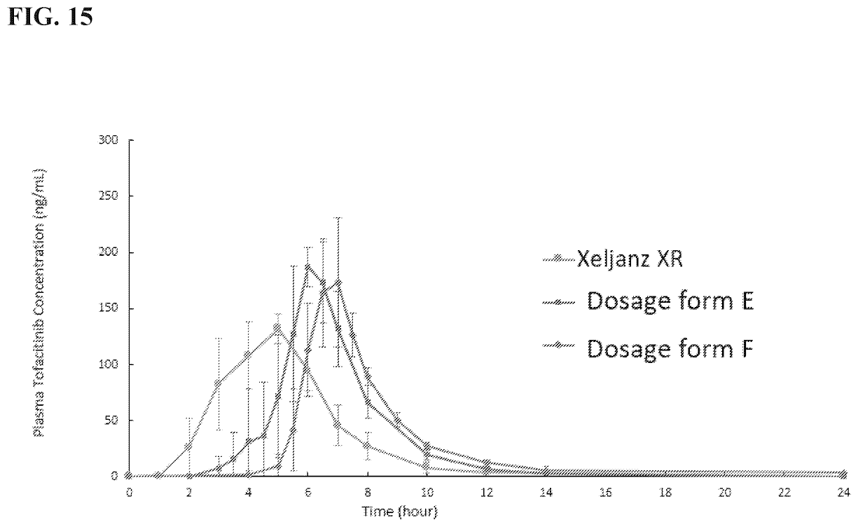

Dosage forms E and F and XELJANZ XR (RLD tablet) were subjected to in vivo pharmacokinetic studies in non-GLP fed male beagle dogs as described in Example 1. The curves of mean plasma concentration for the tested formulations are shown in FIG. 15.

Dosage forms E and F showed effective delay in plasma Cₘₐₓ in vivo. As shown in FIG. 15, both dosage forms E and F achieved desirable, rapid initial increase in plasma drug concentration after a delay. The Tₘₐₓ of dosage form E was delayed by approximately 1 hour compared to XELJANZ XR, the Tₘₐₓ of dosage form F was delayed by approximately 2 hours compared to XELJANZ XR with minimal change of AUC between the two dosage forms.

The in vitro dissolution rates of dosage forms G and H were tested and compared to a marketed tofacitinib containing drug, XELJANZ XR (Pfizer). XELJANZ XR contains 17.77 mg of tofacitinib citrate (also equivalent to 11 mg tofacitinib). Dissolution rates were measured using the same method as described in Example 1.

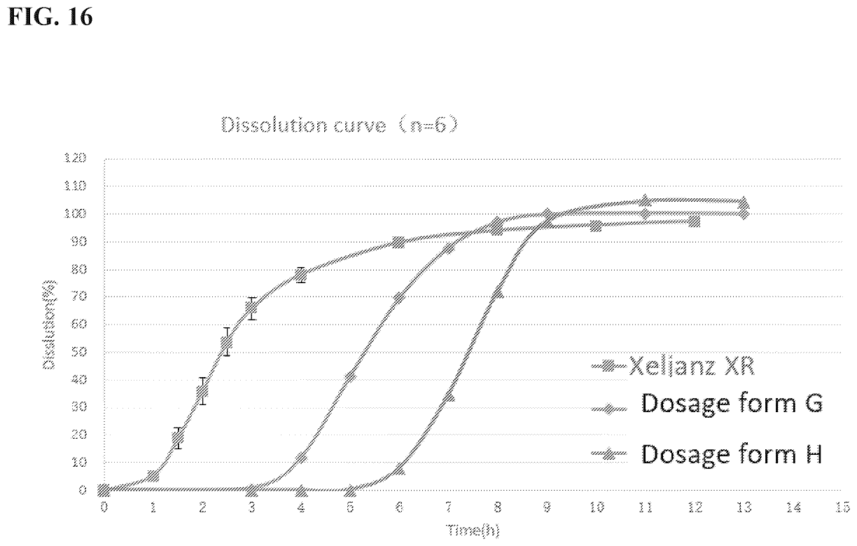

The dissolution profiles of dosage forms G and H in comparison with XELJANZ XR is shown in FIG. 16. Due to the delayed release feature, dosage forms G and H had prolonged dissolution profiles compared to that of XELJANZ XR. XELJANZ XR was 50% dissolved in 2-3 hours, and reached 90% dissolution in 7 hours. Dosage form G reached 50% dissolution in around 6 hours, and 90% between 6-7 hours. Dosage form H reached 50% dissolution between 7-8 hours, and 90% between 8-9 hours. The results are consistent with the thicker delay member on dosage form H (1.2 mm) as compared to the thickness of the delay member on dosage form G (0.8 mm)

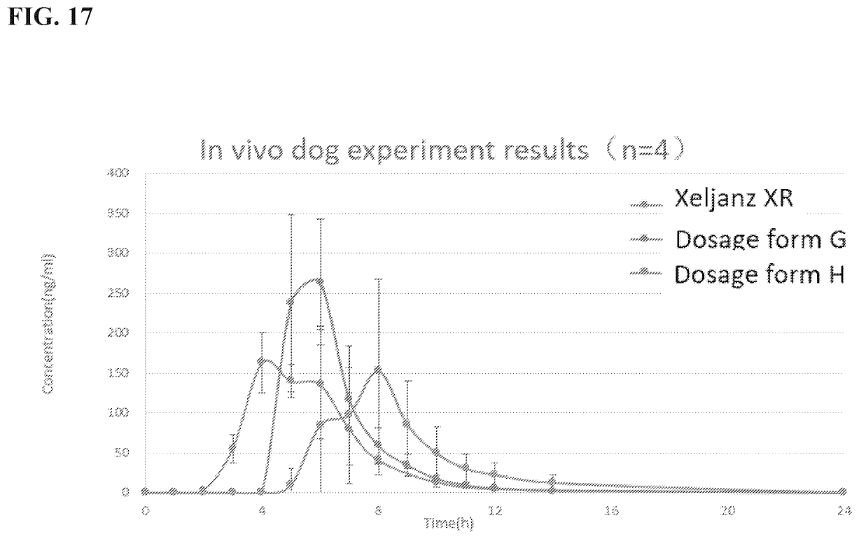

Dosage forms G and H and XELJANZ XR (RLD tablet) were subjected to in vivo pharmacokinetic studies in non-GLP fed male beagle dogs as described in Example 1. The curves of mean plasma concentration for the tested formulations are shown in FIG. 17.

Dosage forms G and H showed effective delay in plasma Cₘₐₓ in vivo. As shown in FIG. 17, both dosage forms G and H achieved desirable, rapid initial increase in plasma drug concentration after a delay. The Tₘₐₓ of dosage form G was delayed by approximately 2 hours compared to XELJANZ XR, the Tₘₐₓ of dosage form H was delayed by approximately 4 hours compared to XELJANZ XR with minimal change of AUC between the two dosage forms.

The in vitro dissolution rates of dosage forms I, J, and K were tested and compared to a marketed tofacitinib containing drug, XELJANZ XR (Pfizer). XELJANZ XR contains 17.77 mg of tofacitinib citrate (also equivalent to 11 mg tofacitinib). Dissolution rates were measured using the same method as described in Example 1.

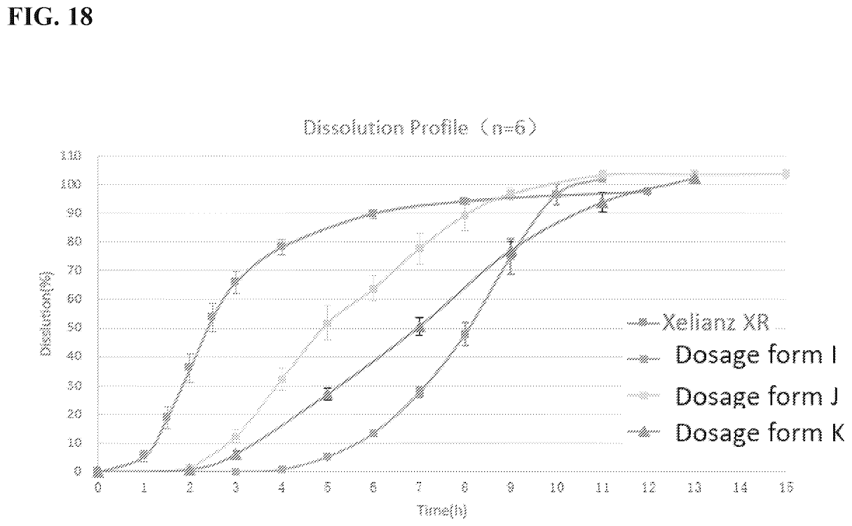

The dissolution profiles of dosage forms I, J, and K in comparison with XELJANZ XR is shown in FIG. 18. Due to the delayed release feature, dosage forms I, J, and K had prolonged dissolution profiles compared to that of XELJANZ XR. XELJANZ XR was 50% dissolved in 2-3 hours, and reached 90% dissolution in 7 hours. Dosage form I reached 50% dissolution around 8 hours, and 90% between 9-10 hours. Dosage form J reached 50% dissolution around 5 hours, and 90% around 8 hours. Dosage form K reached 50% dissolution around 7 hours, and 90% between 9-10 hours.

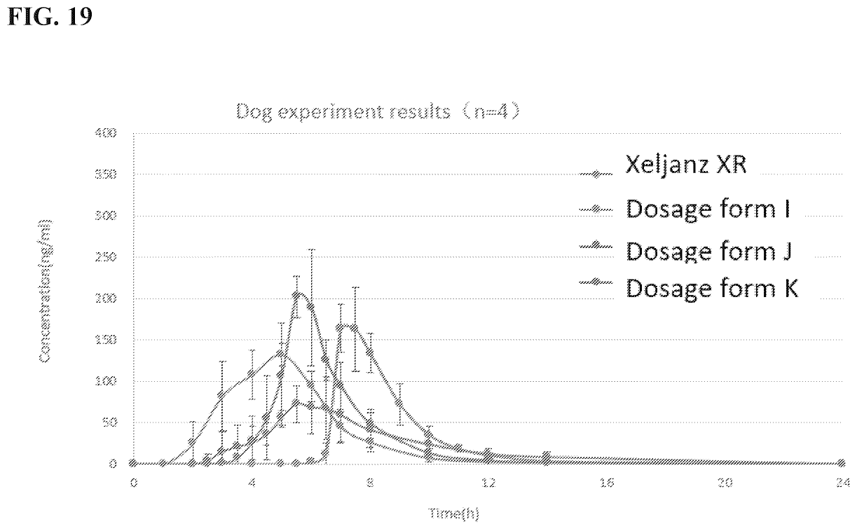

Dosage forms I, J, K, and XELJANZ XR (RLD tablet) were subjected to in vivo pharmacokinetic studies in non-GLP fed male beagle dogs as described in Example 1. The curves of mean plasma concentration for the tested formulations are shown in FIG. 19.

Dosage forms I, J, and K showed effective delay in plasma Cₘₐₓ in vivo. As shown in FIG. 19, dosage forms I, J, K, achieved desirable, rapid initial increase in plasma drug concentration after a delay. The Tₘₐₓ of dosage forms J and K was delayed by approximately 2 hours compared to XELJANZ XR, the Tₘₐₓ of dosage form I was delayed by approximately 4 hours compared to XELJANZ XR with minimal change of AUC between the two dosage forms.

The in vitro dissolution rates of dosage forms L, M, and N were tested and compared to a marketed tofacitinib containing drug, XELJANZ XR (Pfizer). XELJANZ XR contains 17.77 mg of tofacitinib citrate (also equivalent to 11 mg tofacitinib). Dissolution rates were measured using the same method as described in Example 1.

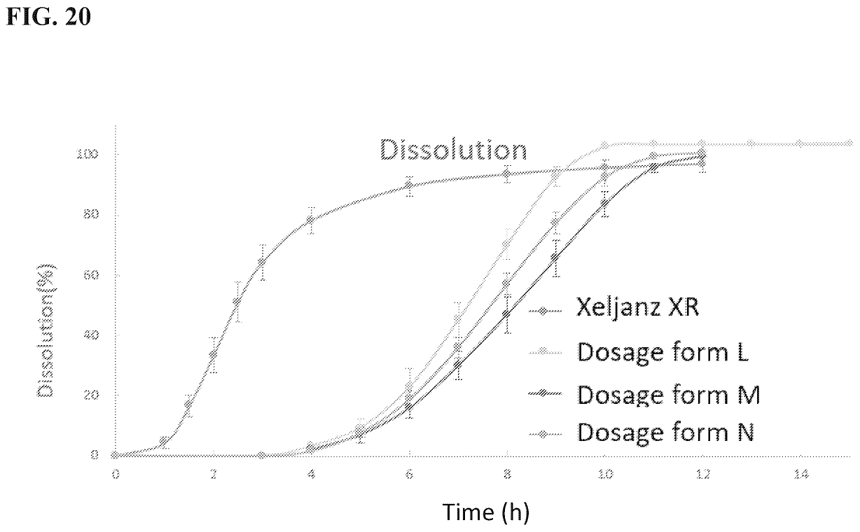

The dissolution profiles of dosage forms L, M, and N in comparison with XELJANZ XR is shown in FIG. 20. Due to the delayed release feature, dosage forms L, M, and N had prolonged dissolution profiles compared to that of XELJANZ XR XELJANZ XR was 50% dissolved in 2-3 hours, and reached 90% dissolution in 7 hours. Dosage form L reached 50% dissolution between 7-8 hours, and 90% between 8-9 hours. Dosage form M reached 50% dissolution between 7-8 hours, and 90% between 9-10 hours. Dosage form N reached 50% dissolution around 8 hours, and 90% between 10-11 hours.

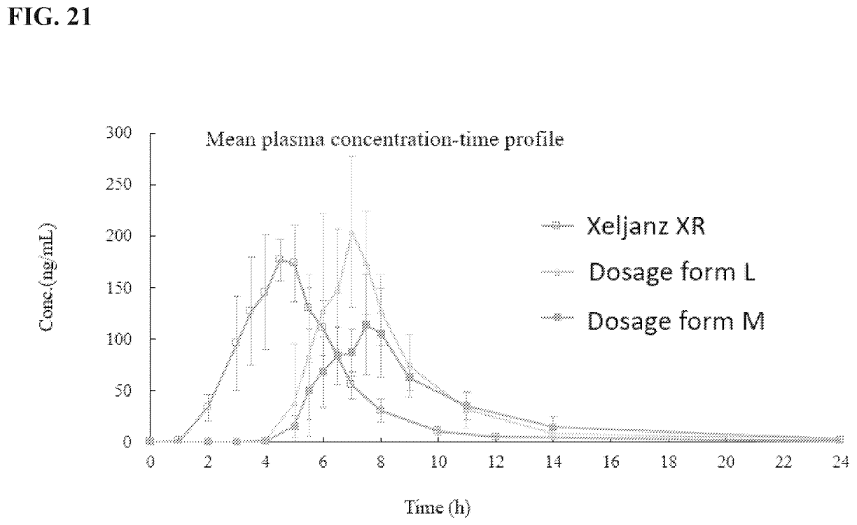

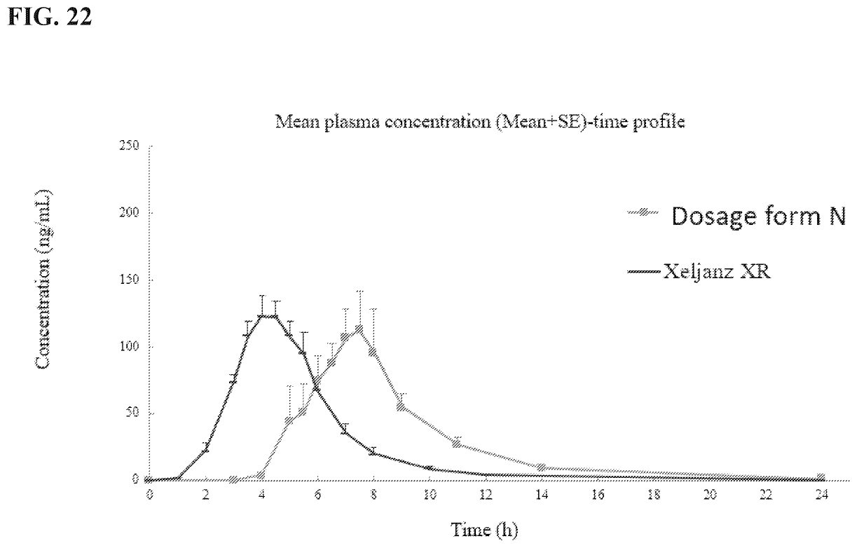

Dosage forms L, M, and N and XELJANZ XR (RLD tablet) were subjected to in vivo pharmacokinetic studies in non-GLP fed male beagle dogs as described in Example 1. The pharmacokinetic curves of dosage forms L and M and Xeljanz XR are shown in FIG. 21. The pharmacokinetic curve for dosage form O and Xeljanz XR are shown in FIG. 22.

Dosage forms L, M, and N showed effective delay in plasma Cₘₐₓ in vivo. As shown in FIGS. 21 and 22, dosage forms L, M, and N achieved desirable, rapid initial increase in plasma drug concentration after a delay. The Tₘₐₓ of dosage forms L, M, and N was delayed by approximately 4 hours compared to XELJANZ XR with minimal change of AUC between dosage forms L, M, and N.

The in vitro dissolution rate of dosage form O was tested. Dissolution rates were measured using the same method as described in Example 1.

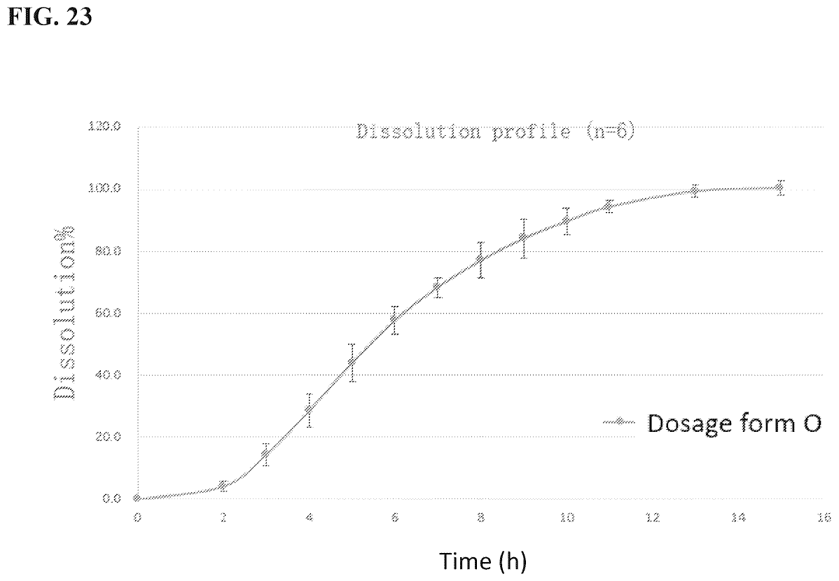

The dissolution profile of dosage form O in comparison with XELJANZ XR is shown in FIG. 23. Dosage form O reached 50% dissolution between 5-6 hours, and 90% between 8-9 hours.

What is claimed is:

1. A delayed sustained-release oral drug dosage form of a Janus kinase (JAK) inhibitor, the delayed sustained-release oral drug dosage form comprising:
a sustained-release drug component comprising a first erodible material admixed with the JAK inhibitor; and
a delay component,
wherein the delay component prevents the release of the JAK inhibitor from the delayed sustained-release oral drug dosage form for about 2 hours to about 6 hours after administration of the delayed sustained-release oral drug dosage form to a human individual.

2. The delayed sustained-release oral drug dosage form of claim 1, wherein the delay component comprises:
a delay member comprising a second erodible material not admixed with the JAK inhibitor; and
a shell,
wherein the delay component completely surrounds the sustained-release drug component.

3. The delayed sustained-release oral drug dosage form of claim 2, wherein the sustained-release drug component is a layer having a top surface and a bottom surface.

4. The delayed sustained-release oral drug dosage form of claim 3, wherein the thickness as measured between the top surface and the bottom surface is substantially consistent.

5. The delayed sustained-release oral drug dosage form of claim 3 or 4, wherein the sustained-release drug component is embedded in the shell such that the bottom surface and a side surface of the sustained-release drug component are in direct contact with the shell.

6. The delayed sustained-release oral drug dosage form of any one of claims 3-5, wherein the top surface of the sustained-release drug component is not in direct contact with the shell.

7. The delayed sustained-release oral drug dosage form of any one of claims 2-6, wherein the delay member is a layer having a top surface and a bottom surface.

8. The delayed sustained-release oral drug dosage form of claim 7, wherein the thickness as measured between the top surface and the bottom surface is substantially consistent.

9. The delayed sustained-release oral drug dosage form of claim 7 or 8, wherein the bottom surface of the delay member, or a portion thereof, is in direct contact with the top layer of the sustained-release drug component.

10. The delayed sustained-release oral drug dosage form of any one of claims 7-9, wherein a side of the delay member is in direct contact with the shell.

11. The delayed sustained release oral drug dosage form of any one of claims 7-10, wherein a portion of the bottom surface of the delay member is in direct contact with the shell.

12. The delayed sustained-release oral drug dosage form of claim 11, wherein the portion of the bottom surface of the delay member that is in direct contact with the shell forms a perimeter extending beyond the top surface of the sustained-release drug component.

13. The delayed sustained-release oral drug dosage form of any one of claims 2-12, wherein the delay member and the shell are configured such that the JAK inhibitor is prevented from being released from the delayed sustained-release oral drug dosage form until after the delay member is eroded.

14. The delayed sustained-release oral drug dosage form of any one of claims 2-13, wherein the shell comprises an insulating material that is impermeable to bodily fluids.

15. The delayed sustained-release oral drug dosage form of claim 14, wherein the insulating material is a non-erodible material.

16. The delayed sustained-release oral drug dosage form of claim 14, wherein the insulating material is an erodible material having a pH-sensitive erosion and/or an erosion rate that allows for the complete release of the JAK inhibitor from the delayed sustained-release oral drug dosage form prior to exposure of the sustained-release drug component to bodily fluids due to erosion of the shell.

17. The delayed sustained-release oral drug dosage form of any one of claims 2-16, wherein the delayed sustained-release oral drug dosage form has a substantially planar top surface.

18. The delayed sustained-release oral drug dosage form of claim 17, wherein the top surface is formed by the delay member and the shell.

19. The delayed sustained-release oral drug dosage form of claim 18, wherein the shell comprises an inset having a depth, wherein the delay member is configured to fit in the inset of the shell.

20. The delayed sustained-release oral drug dosage form of claim 19, wherein the thickness of the delay member is the same as the depth of the inset of the shell.

21. The delayed sustained-release oral drug dosage form of any one of claims 17-20, wherein the top surface is a capsule shape.

22. The delayed sustained-release oral drug dosage form of any one of claims 3-21, wherein the top surface of the sustained-release drug component is a capsule shape.

23. The delayed sustained-release oral drug dosage form of any one of claims 7-22, wherein the top surface of the delay member is a capsule shape.

24. The delayed sustained-release oral drug dosage form of any one of claims 1-23, wherein the delay component prevents the release of the JAK inhibitor from the delayed sustained-release oral drug dosage form for about 2 hours to about 4 hours after administration of the delayed sustained-release oral drug dosage form to a human individual.

25. The delayed sustained-release oral drug dosage form of any one of claims 1-23, wherein the delay component prevents the release of the JAK inhibitor from the delayed sustained-release oral drug dosage form for about 2 hours to about 3 hours after administration of the delayed sustained-release oral drug dosage form to a human individual.

26. The delayed sustained-release oral drug dosage form of any one of claims 1-25, wherein the sustained-release drug component is configured to release the JAK inhibitor from the delayed sustained-release oral drug dosage form according to the following:
(i) not more than 30% of the total JAK inhibitor is released at 1 hour after complete erosion of the delay component or a portion thereof:
(ii) not less than 35% and not more than 75% of the total JAK inhibitor is released at 2.5 hours after complete erosion of the delay component or a portion thereof; and
(iii) not less than 75% of the total JAK inhibitor is released at 5 hours after complete erosion of the delay component or a portion thereof.

27. The delayed sustained-release oral drug dosage form of any one of claims 1-26, wherein the release of the JAK inhibitor is based on an in vitro release rate.

28. The delayed sustained-release oral drug dosage form of any one of claims 1-27, wherein the Tₘₐₓ occurs within about 6 hours after complete erosion of the delay component or a portion thereof.

29. The delayed sustained-release oral drug dosage form of any one of claims 1-28, wherein when administered to the human individual the ratio of geometric mean plasma Cₘₐₓ to Cₘᵢₙ is about 10 to about 100.

30. The delayed sustained-release oral drug dosage form of any one of claims 1-29, wherein the release of the JAK inhibitor is based on an in vitro dissolution technique comprising use of a USP rotating paddle apparatus rotated at about 50 RPM and a test medium comprising 900 mL of 0.05 M potassium phosphate buffer at pH 6.8 and 37° C.

31. The delayed sustained-release oral drug dosage form of any one of claims 3-30, wherein the top surface of the sustained-release drug component has a surface area of about 20 mm² to about 400 mm².

32. The delayed sustained-release oral drug dosage form of any one of claims 3-31, wherein the top surface of the sustained-release drug component has a largest crossing dimension of about 5 mm to about 20 mm.

33. The delayed sustained-release oral drug dosage form of any one of claims 3-32, wherein the top surface of the sustained-release drug component has a crossing dimension perpendicular to a largest crossing dimension of about 2 mm to about 20 mm.

34. The delayed sustained-release oral drug dosage form of any one of claims 3-33, wherein the sustained-release drug component has a thickness of about 0.2 mm to about 5 mm.

35. The delayed sustained-release oral drug dosage form of any one of claims 1-34, wherein the sustained-release drug component has a drug mass fraction (m_{F}) of the JAK inhibitor of about 0.2 to about 0.6.

36. The delayed sustained-release oral drug dosage form of any one of claims 1-35, wherein the sustained-release drug layer has an in vitro dissolution rate of about 2% per hour to about 40% per hour based on an in vitro dissolution technique comprising use of a USP rotating paddle apparatus rotated at about 50 RPM and a test medium comprising 900 mL of 0.05 M potassium phosphate buffer at pH 6.8 and 37° C.

37. The delayed sustained oral drug dosage form of any one of claims 1-36, wherein the first erodible material of the sustained-release drug component comprises one or more of hydroxypropyl cellulose (HPC EF), vinylpyrrolidone-vinyl acetate copolymer (VA64), triethyl citrate (TEC), and glycerin.

38. The delayed sustained oral drug dosage form of any one of claims 1-37, wherein the first erodible material of the sustained-release drug component comprises HPC EF at about 35 w/w % to about 45 w/w %, VA64 at about 5 w/w % to about 15 w/w %, and glycerin at about 10 w/w % to about 20 w/w %.

39. The delayed sustained-release oral drug dosage form of any one of claims 7-38, wherein the top surface of the delay member has a surface area of about 20 mm² to about 400 mm².

40. The delayed sustained-release oral drug dosage form of any one of claims 7-39, wherein the top surface of the delay member has a largest crossing dimension of about 5 mm to about 20 mm.

41. The delayed sustained-release oral drug dosage form of any one of claims 7-40, wherein the top surface of the delay member has a crossing dimension perpendicular to a largest crossing dimension of about 2 mm to about 20 mm.

42. The delayed sustained-release oral drug dosage form of any one of claims 7-41, wherein the delay member has a thickness of about 0.2 mm to about 5 mm.

43. The delayed sustained-release oral drug dosage form of any one of claims 2-42, wherein the delay completely dissolves with in about 6 hours after administration of the delayed sustained-release oral drug dosage form to the human individual.

44. The delayed sustained-release oral drug dosage form of any one of claims 2-43, wherein the second erodible material of the delay layer comprises one or more of hydroxypropyl cellulose (HPC EF), triethyl citrate (TEC), and titanium dioxide.

45. The delayed sustained-release oral drug dosage form of any one of claims 2-44, wherein the delay layer comprises HPC EF at about 80 w/w % to about 90 w/w %, TEC at about 10 w/w % to about 20 w/w %, and titanium dioxide at about 0.1 w/w % to about 0.3 w/w %.

46. The delayed sustained-release oral drug dosage form of any one of claims 2-45, wherein the shell has a largest crossing dimension of about 5 mm to about 20 mm.

47. The delayed sustained-release oral drug dosage form of any one of claims 2-46, wherein the shell has a crossing dimension perpendicular to a largest crossing dimension of about 5 mm to about 20 mm.

48. The delayed sustained-release oral drug dosage form of any one of claims 2-47, wherein the delayed sustained-release oral drug dosage form has a thickness of about 0.2 mm to about 15 mm.

49. The delayed sustained-release oral drug dosage form of any one of claims 2-48, wherein the shell has a minimum thickness of at least about 0.4 mm.

50. The delayed sustained release oral drug dosage form of any one of claims 2-49, wherein the shell comprises one or more of ammonio methacrylate copolymer type B, ethylcellulose, stearic acid, and titanium dioxide.

51. The delayed sustained-release oral drug dosage form of any one of claims 2-50, wherein the shell comprises ammonio methacrylate copolymer type B at about 60 w/w % to about 70 w/w %, ethylcellulose at about 10 w/w % to about 20 w/w %, stearic acid at about 15 w/w % to about 25 w/w %, and titanium dioxide at about 0.1 w/w/% to about 0.3 w/w %.

52. The delayed sustained-release oral drug dosage form of any one of claims 1-51, wherein the JAK inhibitor interferes with the JAK-STAT signaling pathway.

53. The delayed sustained-release oral drug dosage form of any one of claims 1-52, wherein the JAK inhibitor is an inhibitor of any one or more of JAK1, JAK2, JAK3, or TYK2.

54. The delayed sustained-release oral drug dosage form of any one of claims 1-53, wherein the JAK inhibitor is tofacitinib or a pharmaceutically acceptable salt thereof.

55. The delayed sustained-release oral drug dosage form of any one of claims 1-54, wherein the JAK inhibitor is tofacitinib citrate.

56. The delayed sustained-release oral drug dosage form of any one of claims 1-55, wherein the amount of the JAK inhibitor in the delayed sustained-release oral drug dosage form is about 11 mg.

57. The delayed sustained-release oral drug dosage form of any one of claims 1-56, wherein the amount of the JAK inhibitor in the delayed sustained-release oral drug dosage form is about 22 mg.

58. The delayed sustained-release oral drug dosage form of any one of claims 1-57, wherein the delayed-sustained-release oral drug dosage form is not an osmotic-controlled release oral drug dosage form.

59. A commercial batch of a delayed sustained-release oral drug dosage form of any one of claims 1-58, wherein the commercial batch has a standard deviation of about 0.05 or less for each of the following:
an amount of a JAK inhibitor in the delayed sustained-release oral drug dosage form;
weight of the delayed sustained-release oral drug dosage form;
a largest crossing dimension of the delayed sustained-release oral drug dosage form; and
a crossing dimension perpendicular to the largest crossing dimension of the delayed sustained-release oral drug dosage form.

60. The commercial batch of claim 59, wherein the commercial batch comprises at least about 1000 of the delayed sustained-release oral drug dosage forms.

61. A method of three-dimensional (3D) printing of a delayed sustained-release oral drug dosage form of any one of claims 1-58, the method comprising dispensing materials according to a layer-by-layer model of the delayed sustained-release oral drug dosage form to print the delayed sustained-release oral drug dosage form, wherein each layer of the layer-by-layer model is printed by dispensing, as necessary, for a layer:
(a) a sustained-release drug component comprising a first erodible material admixed with a JAK inhibitor;
(b) a delay member comprising a second erodible material not admixed with the JAK inhibitor; and
(c) a shell.

62. The method of claim 61, further comprising generating the layer-by-layer model of the delayed sustained-release oral drug dosage form.

63. The method of claim 61 or 62, wherein the dispensing is via melt extrusion deposition (MED).

64. The method of one of claims 61-63, wherein dispensing of each material is performed by a different printing head.

65. A method for preparing a delayed sustained-release tofacitinib oral drug dosage form by three-dimensional (3D) printing,
wherein the delayed sustained-release tofacitinib oral drug dosage form comprises a shell containing an insoluble material, a pharmaceutical core containing tofacitinib, and a delay member without tofacitinib,
the method comprising dispensing materials according to a layer-by-layer model of the delayed sustained-release oral drug dosage form to print the delayed sustained-release oral drug dosage form, wherein each layer of the layer-by-layer model is printed by dispensing, as necessary, for a layer:
(a) a pharmaceutical core containing tofacitinib;
(b) the delay member without tofacitinib; and
(c) the shell comprising an insoluble material.

66. The method of claim 65, wherein the dispensing is via melt extrusion deposition (MED).

67. The method of claim 65 or 66, wherein the dispensing of each material is performed by a different printing head.

68. A method of injection molding an oral drug dosage form of any one of claims 1-58, the method comprising:
(a) injecting a hot melt of the shell material into a mold cavity to form the shell;
(b) injecting a hot melt of the first erodible material admixed with a JAK inhibitor into the shell to form the sustained-release drug component; and
(c) injecting a hot melt of the second erodible material not admixed with the JAK inhibitor into the shell to form the delay member.

69. A method for preventing morning stiffness caused by rheumatoid arthritis, the method comprising administering to a human individual a delayed sustained-release oral drug dosage form of any one of claims 1-58, wherein the delayed sustained-release oral drug dosage form is administered within about 1 hour of going to bed.

70. A method for preventing morning stiffness caused by psoriatic arthritis, the method comprising administering to a human individual a delayed sustained-release oral drug dosage form of any one of claims 1-58, wherein the delayed sustained-release oral drug dosage form is administered within about 1 hour of going to bed.

71. A method for treating ulcerative colitis, the method comprising administering to a human individual a delayed sustained-release oral drug dosage form of any one of claims 1-58.
